(19) United States
(12) Patent Application Publication
(10) Pub. No.: US 2026/0355397 A1
(43) Pub. Date: Nov. 12, 2026

(54) MODULAR BREAST PUMP SYSTEM AND COMPONENTS AND USES THEREOF

(71) Applicant: Nooshee Inc., Raleigh, NC (US)

(72) Inventors: Laura Haft, Raleigh, NC (US); William C. Brody, Raleigh, NC (US); Heath Stephenson, London (GB)

(73) Assignee: Nooshee Inc., Raleigh, NC (US)

(21) Appl. No.: 19/515,092

(22) PCT Filed: Aug. 30, 2024
(86) PCT No.: PCT/US2024/044910

§ 371 Date: Feb. 27, 2026

Related U.S. Application Data
(60) Provisional application No. 63/535,808, filed on Aug. 31, 2023.

(51) Int. Cl. A61M 1/06

(52) U.S. Cl. CPC A61M 1/064; A61M 2205/127; A61M 2209/088

(57) ABSTRACT

A modular breast pump system includes a breast cup, a base module, a portable module, and a removable pumping unit. The base module includes a base enclosure and a removable reservoir within the base enclosure. The portable module includes a portable reservoir. The removable pumping unit is configured to be removably coupled with each of the base module and the portable module individually. The removable pumping unit includes a pressure source in fluidic communication with the breast cup and configured to apply pressure to the breast cup that induces lactation. When the removable pumping unit is coupled to the base module, the pressure source is configured to force milk from the breast cup into the removable reservoir. When the removable pumping unit is coupled to the portable module, the pressure source is configured to force milk from the breast cup into the portable reservoir

### CROSS-REFERENCE TO RELATED APPLICATION

This application claims the benefit of and priority to U.S. Provisional Patent Application No. 63/535,808, filed on Aug. 31, 2023, which is incorporated herein by reference in its entirety.

### BACKGROUND

New mothers use breast pumps post-birth in order to accomplish milk feeding and milk expression. Breast pumps systems draw breast milk from a breast of a user and may be used to pump breast milk for later consumption by an infant, to stimulate lactation in users with low milk supply, or to relieve engorgement. Breast pumps may be manually operated, for example by squeezing a handle or operation of a foot pedal. Breast pumps may also be electrically driven by a drive unit.

On average, new mothers feed their infants more than 12 times per day, with newborn infants often needing to be fed every two to three hours. Mothers or other care providers may spend more than four hours each day breastfeeding, on average spending 20 minutes out of every two hours breastfeeding, interrupting work, social, and sleep schedules.

### SUMMARY

Traditional breast pumps demand mothers to be stationary and near an outlet to draw the power required to fully express the mother's milk. Consequently, portable breast pumps are often less powerful and are unable to fully express all the milk in a mother's breast. Not fully emptying breasts during pumping sessions can contribute to reduced milk supply, and clogged milk ducts, leading to mastitis.

Commercially available portable breast pumps also include on-body milk collection, which require a user to remove the breast cups in order to access the milk, and many of these pumps also have a large breast cup and/or milk collection profile. Breast cup removal and large breast cup profiles do not allow for discreet public pumping. Thus, there is a need for discreet, wearable, portable breast pump systems that provide all the power and benefits of traditional breast pumps, and permit access to pumped milk without necessitating breast cup removal, while allowing the user the mobility to pump while navigating daily activities or traveling.

The demands of breastfeeding and pumping require mothers to wake up throughout the night drastically decreasing the total hours of sleep per night a mother is able to achieve. Such sleep loss contributes to a number of conditions in women, such as mastitis and postpartum depression.

There are no commercially available breast pump systems configured for serial breast pumping sessions without removal of a breast cup or without replacement of a reservoir in the system

The present disclosure provides improved modular breast pumps, components thereof, and methods of use thereof that address these and other problems of traditional breast pumps.

One embodiment relates to a breast pump system including a breast cup, a base module, a portable module, and a removable pumping unit. The base module includes a base enclosure and a removable reservoir within the base enclosure. The portable module includes a portable reservoir. The removable pumping unit is configured to be removably coupled with each of the base module and the portable module individually. The removable pumping unit includes a pressure source in fluidic communication with the breast cup and configured to apply pressure to the breast cup that induces lactation. When the removable pumping unit is coupled to the base module, the pressure source is configured to force milk from the breast cup into the removable reservoir. When the removable pumping unit is coupled to the portable module, the pressure source is configured to force milk from the breast cup into the portable reservoir.

Another embodiment relates to a method of breast pumping. The method includes attaching a breast cup to a human breast of a subject. The method further includes coupling a removable pumping unit to one of a base module or a portable module of a modular breast pump system, the removable pumping unit having a pressure source. The method further includes applying pressure from the pressure source to the breast cup to pump milk from the human breast and into a first reservoir of the one of the base module or the portable module. The method further includes coupling the removable pumping unit to the other of the base module or the portable module. The method further includes applying pressure from the pressure source to the breast cup to pump milk from the human breast and into a second reservoir of the other of the base module or the portable module.

The present disclosure provides a modular breast pump system including: (a) a base module; and (b) a portable module, further including a negative pressure source, wherein the base and/or portable modules are configured to be in fluidic connection with a breast cup. In some embodiments, the modular breast pump system also includes (c) a reservoir enclosure in (a) the base module and/or (b) the portable module. In some embodiments, the base and/or portable module also includes (d) a reservoir configured to be in fluidic communication with a breast cup. In some embodiments, the modular breast pump system also includes (e) a milk distribution system disposed in (a) the base module. In some embodiments, the system includes the breast cup.

In one aspect, the base module and portable module may be physically releasably connected. In some embodiments, the base module and portable module may be electrically releasably connected.

In some embodiments, the portable module may include all components required to perform a breast pumping session. In some embodiments, the base and/or portable module may include all components required to perform a breast pumping session.

In some embodiments, the modular breast pump system includes a positive pressure source. In some embodiments, the negative pressure source is the same as the positive pressure source. In some embodiments, the pressure source is a pump. In further embodiments, the pump is a vacuum pump.

In some embodiments, the pressure source can provide a negative pressure of about 0 mmHg to about 400 mmHg. In some embodiments the pressure source can provide a positive pressure of about 0 mmHg to about 400 mmHg.

In some embodiments, the modular breast pump system can be considered to be hospital grade. In some embodiments the modular breast pump system can be considered to be personal grade.

In some embodiments, the base and/or portable module includes a first fluidic conduit providing fluidic communication between the pressure source and a breast cup. In some embodiments, the first fluidic conduit releasably attaches to the pressure source and/or the breast cup. In some embodiments, the base and/or portable module further includes a second fluidic conduit providing fluidic communication between a breast cup and a reservoir. In some embodiments, the second fluidic conduit releasably attaches to the breast cup and/or a reservoir.

In some embodiments, the modular breast pump system further includes a drive unit operatively connected to the negative pressure source and/or the positive pressure source. In some embodiments, the drive unit includes the control unit.

In some embodiments, the modular breast pump system further includes a power source. In some embodiments, the base and/or portable module includes a power source. In some embodiments, the base module includes a first power source, and the portable module includes a second power source. In some embodiments, the power source is a battery. In some embodiments, the power source optionally includes a rechargeable battery or a single use battery. In some embodiments, the base module and the portable module may be configured to interface together, such that when the base module and portable module are connected, the first power source in the base module may charge the second power source in the portable module. In some embodiments, the base module and the portable module may be configured to interface together, such that when the base module and portable module are connected, power is drawn from the first power source in the base module. In some embodiments, the modular breast pump system further includes a button, icon, display, timer, alarm, and/or power switch.

In some embodiments, the modular breast pump system further includes a reservoir. In some embodiments, the modular breast pump system includes a plurality of reservoirs. In some embodiments, the reservoir further includes a cooling element and/or insulation. In some embodiments, the reservoir further includes a pressure outlet. In some embodiments, the reservoir further includes a unique tag.

In some embodiments, each reservoir includes a pressure sensor.

In some embodiments, each reservoir includes a weight sensor, e.g., a scale.

In some embodiments, the modular breast pump system further includes a sensor including one or more of a weight sensor, a load cell, a temperature sensor, a pressure sensor, an optical sensor, a flow sensor, a volume sensor, a pH sensor, and a viscosity sensor. In some embodiments, a single weight sensor, e.g., a scale, is employed for multiple reservoirs, e.g., all reservoirs.

In some embodiments, the modular breast pump system further includes a valve. In some embodiments, the modular breast pump further includes a plurality of valves. In some embodiments, the pressure source includes a valve. In some embodiments, the pressure source includes a plurality of valves. In some embodiments, the breast cup further includes a valve. In some embodiments, the first fluidic conduit and/or the second fluidic conduit further include a valve or a plurality of valves.

In some embodiments, the portable module is configured to be carried by a subject. In some embodiments, the portable module includes a handle. In some embodiments, the portable module includes a strap and or a clip. In some embodiments, the portable module is configured to be releasably attached to a subject's body.

In some embodiments, the modular breast pump system further includes a garment, and a breast cup is releasably secured to the garment. In some embodiments, the garment is a bra or shirt. In some embodiments, the coefficient of friction between the garment and the housing is less than 0.4 (e.g., 0.3, 0.2, 0.1, or 0.5). In some embodiments, the portable module is further secured to the garment.

In some embodiments, the modular breast pump system further includes an enclosure. In some embodiments, the enclosure may be disposed of inside the base and/or portable module. In some embodiments, a reservoir may be disposed of inside of the enclosure. In some embodiments a plurality of reservoirs may be disposed of inside of the enclosure. In some embodiments, the base module may include a first reservoir enclosure, and the portable module may include a second reservoir enclosure. In some embodiments, the first reservoir enclosure and the second reservoir enclosure may be releasably connected, such that when the base and portable module are mated it includes one reservoir enclosure. In some embodiments, the enclosure includes a cooling element and/or insulation.

In some embodiments, a reservoir may be releasably connected to the portable module. In some embodiments, the reservoir may include internal and/or external threads, a catch mechanism, or other appropriate attachment mechanism, such that it may be releasably attached to the portable module. In some embodiments, the reservoir may be releasably attached to the portable module via a latch, snap fit, or other mechanism.

In some embodiments, the subject is moving, resting, sleeping, or traveling.

In some embodiments, the breast pump system further includes a plurality of reservoirs.

In some embodiments, the breast pump system further includes a pressure outlet in fluidic communication with each reservoir. In some embodiments, each pressure outlet includes a valve. In some embodiments, the pressure outlet is a breather tube.

In some embodiments, the breast pump system further includes a manifold disposed between the milk chamber and the plurality of reservoirs. In some embodiments, the manifold includes a straight manifold, a right-angle manifold, a round manifold, a block manifold, a square manifold, a hex manifold, a wye manifold, or a rotating joint manifold.

In some embodiments, the manifold includes a valve.

In some embodiments, the manifold includes an inlet and an outlet that is configured to move from a first position to a second position, e.g., to provide fluidic communication with two different reservoirs.

In some embodiments, the outlet of the manifold is configured to be in fluidic communication with one reservoir at a time.

In some embodiments, the manifold includes a plurality of outlets. In some embodiments, each reservoir includes an inlet in fluidic communication with a different outlet of the manifold. In some embodiments, each outlet includes a valve. In some embodiments, the valve is a solenoid valve.

In some embodiments, the manifold includes (i) a moveable, e.g., rotating, member including the inlet of the manifold and a fluid conduit, wherein the inlet of the manifold and the fluid conduit are in fluidic communication, and (ii) a stationary member including the plurality of outlets of the manifold. In some embodiments, the moveable, e.g., rotating, member is configured to move, e.g., rotate, and align the fluid conduit with each of the plurality of outlets of the manifold. In some embodiments, the moveable, e.g., rotating, member is axially arranged within the stationary member.

In some embodiments, the modular breast pump system includes a breast milk distribution system. In some embodiments, the breast milk distribution system is disposed of within the base module.

In some embodiments, the breast milk distribution system further includes a plurality of reservoirs, wherein each of the plurality of reservoirs are configured to be in fluidic communication with a different outlet. In some embodiments, the breast milk distribution system further includes from 2 to 12 reservoirs. In some embodiments, the breast milk distribution system further includes from 4 to 6 reservoirs.

In some embodiments, each reservoir includes a pressure sensor.

In some embodiments, the breast milk distribution system further includes a pressure outlet in fluidic communication with each reservoir. In some embodiments, each pressure outlet includes a valve. In some embodiments, the pressure outlet is a breather tube.

In some embodiments, each reservoir includes an inlet in fluidic communication with a different outlet of the manifold. In some embodiments, the inlet of the reservoir is configured to open and close.

In some embodiments, the breast milk distribution system further includes a plurality of valves, wherein the inlet of each reservoir includes a valve, e.g., to seal each inlet when not being filled.

In some embodiments, each reservoir includes a lid, wherein lid includes the pressure outlet, the inlet of the reservoir, and/or the valve of the reservoir.

In some embodiments, the breast milk distribution system further includes a pressure source in fluidic communication with the breast cup, the manifold, and/or at least one of the plurality of reservoirs. In some embodiments, the pressure source is a negative pressure source or a positive pressure source. In some embodiments, the pressure source is configured to open and/close an outlet of the manifold. In some embodiments, the pressure source is configured to open and/close an inlet of a reservoir.

In some embodiments, the manifold includes a straight manifold, a right-angle manifold, a round manifold, a block manifold, a square manifold, a hex manifold, a wye manifold, or a rotating joint manifold.

In some embodiments, the manifold includes (i) a moving, e.g., rotating, member including the inlet of the manifold and a fluid conduit, wherein the inlet of the manifold and the fluid conduit are in fluidic communication, and (ii) a stationary member including the plurality of outlets of the manifold. In some embodiments, the moving, e.g., rotating, member is configured to move, e.g., rotate, and align the fluid conduit with each of the plurality of outlets of the manifold.

In some embodiments, the fluid conduit of the moving, e.g., rotating, member includes an outlet, and the diameter of the outlet of the fluid conduit has the same diameter as the outlets of the manifold. In some embodiments, the diameter of the outlet of the fluid conduit is larger than the diameter of the outlets of the manifold. In some embodiments, the diameter of the outlet of the fluid conduit is smaller than the diameter of the outlets of the manifold.

In some embodiments, the outlets of the manifold are radially disposed in the stationary member.

In some embodiments, the inlet of the manifold has a longitudinal axis orthogonally arranged to a longitudinal axis of each of the outlets of the manifold.

In some embodiments, each of outlets of the manifold includes a valve. In some embodiments, the valve includes a ball valve, a batch dispensing valve, a butterfly valve, a diaphragm valve, a diverting valve, a gate valve, a piston valve, a plug valve, a pinch valve, a saddle valve, a solenoid valve, a stem valve, a stop cock valve, or a three-way valve. In some embodiments, the valve is a solenoid valve. In some embodiments, the valve is a pinch valve. In some embodiments, the valve is a three-way solenoid pinch valve.

In some embodiments, the breast milk distribution system further includes a drive unit configured to open and close the valve. In some embodiments, the drive unit is a stepper motor or a linear actuator. In some embodiments, the stepper motor is attached to the manifold, wherein the stepper motor is configured to rotate the manifold. In some embodiments, the linear actuator is an electric linear actuator, a hydraulic linear actuator, or a pneumatic linear actuator.

In some embodiments, the breast milk distribution system further includes a fluid conduit providing fluidic communication between a breast cup and the inlet of the manifold. In some embodiments, the breast milk distribution system further includes a plurality of fluid conduits providing fluidic communication between each of the outlets of the manifold and the plurality of reservoirs.

In some embodiments, the plurality of reservoirs is disposed in an enclosure. In some embodiments, the enclosure includes a lid, e.g., wherein the manifold is disposed in the lid of the enclosure. In some embodiments, closure of the lid is configured to provide fluidic communication between at least one reservoir and at least one outlet of the manifold. In some embodiments, the enclosure further includes the pressure source. In some embodiments, the enclosure includes a cooling element and/or insulation. In some embodiments, the enclosure includes a weight sensor, e.g., a scale.

In some embodiments, the breast milk distribution system further includes a control unit configured to (i) send a signal to the valve to open and close or (ii) send a signal to the drive unit to move the moving, e.g., rotating, member.

In some embodiments, the present disclosure provides a breast milk distribution system, including: a breast cup configured to receive a nipple; and a manifold in fluidic communication with the breast cup, wherein the manifold includes an inlet and an outlet that is configured to move from a first position to a second position.

In some embodiments, the breast milk distribution system further includes a plurality of reservoirs, wherein each of the plurality of reservoirs is configured to be in fluidic communication with the outlet of the manifold. In some embodiments, the outlet of the manifold is configured to be in fluidic communication with one reservoir at a time.

In some embodiments, each reservoir includes an inlet, and the inlet of the reservoir is configured to open and close. In some embodiments, each inlet of each reservoir includes a valve configured to open and close the inlet.

In some embodiments, each reservoir includes a pressure sensor.

In some embodiments, each reservoir includes a pressure outlet.

In some embodiments, the breast milk distribution system further includes a pressure source in fluidic communication with the breast cup, the manifold, and/or at least one of the plurality of reservoirs. In some embodiments, the pressure source is a negative pressure source or a positive pressure source.

In some embodiments, the breast cup includes (i) a housing; (ii) a breast shield; and (iii) a diaphragm disposed between the housing and the breast shield, wherein the diaphragm is further disposed between a pressure chamber and a milk chamber. In some embodiments, the negative pressure source is fluidic communication with the pressure chamber. In some embodiments, the plurality of reservoirs is configured to be in fluidic communication with the milk chamber.

In some embodiments, the breast milk distribution system further includes a drive unit configured to move the outlet. In some embodiments, the drive unit is a stepper motor or a linear actuator. In some embodiments, the manifold includes the stepper motor, and the stepper motor is configured to move, e.g., radially, the outlet of the manifold. In some embodiments, the linear actuator is an electric linear actuator, a hydraulic linear actuator, or a pneumatic linear actuator.

In some embodiments, the breast milk distribution system further includes a fluid conduit providing fluidic communication between the breast cup and the inlet of the manifold. In some embodiments, the breast milk distribution system further includes a fluid conduit providing fluidic communication between the inlet of the manifold and the outlet of the manifold.

In some embodiments, the breast milk distribution system further includes an arm, wherein the fluid conduit is disposed in and/or on the arm. In some embodiments, the arm includes an articulating arm.

In some embodiments, the plurality of reservoirs is disposed in an enclosure. In some embodiments, the enclosure includes a lid, and wherein the manifold is disposed in the lid of the enclosure.

In some embodiments, the breast milk distribution system further includes a hall effect sensor.

In some embodiments, the breast milk distribution system further includes a microswitch, e.g., to rehome the moving member.

The present disclosure provides a method of distributing milk from breast pumping, including: (i) providing the breast milk distribution system of the present disclosure; (ii) pumping a human breast to induce milk from a first breast pumping session to flow from a nipple to the manifold via a breast cup; (iii) providing fluidic communication between the inlet of the manifold and a first reservoir to allow milk from the first breast pumping session to flow into the first reservoir; (iv) removing fluidic communication between the inlet of the manifold and the first fluid reservoir; (v) pumping the human breast to induce milk from a second breast pumping session to flow from the nipple to the manifold via the breast cup; and (vi) providing fluidic communication between the inlet of the manifold and a second reservoir to allow milk from the second breast pumping session to flow into the second reservoir.

In some embodiments, providing fluidic communication between the inlet of the manifold and the first reservoir includes opening a first valve. In some embodiments, the control unit sends a signal to the first valve to open and/or close. In some embodiments, the control unit sends a signal to the second valve to open and/or close.

In some embodiments, the method further includes from 30 minutes to 240 minutes (about 30 minutes to 60 minutes, about 30 minutes to 90 minutes, about 60 minutes to about 180 minutes, about 60 minutes to about 120 minutes, about 90 minutes to 120 minutes, or about 120 minutes to 240 minutes) between step (ii) and step (v).

In some embodiments, the method further includes (vi) removing fluidic communication between the inlet of the manifold and the second fluid reservoir; (vii) providing fluidic communication between the inlet of the manifold and a third reservoir; and (viii) pumping the human breast to induce milk from a third breast pumping session to flow from the nipple to the manifold via the breast cup. In some embodiments, the method further includes from 30 minutes to 240 minutes (about 30 minutes to 60 minutes, about 30 minutes to 90 minutes, about 60 minutes to about 180 minutes, about 60 minutes to about 120 minutes, about 90 minutes to 120 minutes, or about 120 minutes to 240 minutes) between step (v) and step (viii).

In some embodiments, the method further includes providing fluidic communication between (i) the inlet of the manifold and (ii) the first reservoir, the second reservoir, or the third fluid reservoir by moving, e.g., rotating, the moving, e.g., rotating, member. In some embodiments, the method further includes removing fluidic communication between (i) the inlet of the manifold and (ii) the first reservoir, the second reservoir, or the third fluid reservoir by moving, e.g., rotating, the moving, e.g., rotating, member.

In some embodiments, the method further includes providing fluidic communication between (i) the inlet of the manifold and (ii) the first reservoir, the second reservoir, or the third fluid reservoir by opening the valve to the inlet of the first reservoir, the second reservoir, or the third fluid reservoir. In some embodiments, the method further includes removing fluidic communication between (i) the inlet of the manifold and (ii) the first reservoir, the second reservoir, or the third fluid reservoir by closing the valve to the inlet of the first reservoir, the second reservoir, or the third fluid reservoir.

In some embodiments, the method further includes providing fluidic communication between (i) the inlet of the manifold and (ii) the first reservoir, the second reservoir, or the third fluid reservoir by moving the arm. In some embodiments, the method further includes removing fluidic communication between (i) the inlet of the manifold and (ii) the first reservoir, the second reservoir, or the third fluid reservoir by moving the arm. In some embodiments, the method further includes moving the arm with the drive unit, wherein the drive unit is the stepper motor.

In some embodiments, the breast cup includes (a) a housing; (b) a breast shield; and
(c) a diaphragm disposed between the housing and the breast shield. The diaphragm is disposed between a pressure chamber and a milk chamber.

In some embodiments, the breast shield includes a wide portion and a nipple tunnel.

In some embodiments, the breast cup further includes a milk chamber wall disposed between the diaphragm and the breast shield. The diaphragm and the milk chamber wall define the milk chamber.

In some embodiments, the milk chamber includes an inlet from the breast shield, and the diaphragm releasably seals the inlet. In some embodiments, the inlet includes a valve, e.g., a mechanical valve, an umbrella valve, a butterfly valve, a disk valve, or a duckbill valve. In some embodiments, the inlet comprises a plurality of orifices. In some embodiments, the plurality of orifices is circumferentially arranged at a nipple tunnel of the breast shield.

In some embodiments, the breast shield and/or the housing includes ridges, bumps or dimples. In some embodiments, the breast shield includes a toroidal ridge.

In some embodiments, the diaphragm includes a material having a Shore hardness from A10 to A80 or D10 to D80. In some embodiments, the diaphragm includes a first material and a second material, where the second material is more rigid than the first material and seals the inlet.

In some embodiments, the housing, diaphragm, and/or breast shield includes a clear material. In some embodiments, the clear material forms a magnifying lens. In some embodiments, the diaphragm includes the clear material where the diaphragm contacts an inlet to the milk chamber.

In some embodiments, the diaphragm is shaped to conform to the shape of the housing or breast shield.

In some embodiments, the milk chamber further comprises an outlet.

In some embodiments, the breast cup further includes a one directional valve configured to allow fluid flow from the milk chamber to a reservoir. In some embodiments, the one directional valve is disposed in the outlet.

In one aspect, the present disclosure provides a modular breast pump system including: (a) a breast cup as described herein; (b) a negative pressure source, wherein the negative pressure source is in fluidic communication with the pressure chamber; and (c) optionally a reservoir in fluidic communication with the milk chamber. In some embodiments, the system includes the reservoir.

In some embodiments, the modular breast pump system further includes a positive pressure source, wherein the positive pressure source is in fluidic communication with the pressure chamber. In some embodiments, the positive pressure source is a vacuum pump. In some embodiments, a pressure source includes the negative pressure source and the positive pressure source. In some embodiments, the negative pressure source is a vacuum pump.

In some embodiments, the modular breast pump system further includes a first fluid conduit providing fluidic communication between the milk chamber and the reservoir. In some embodiments, the first fluid conduit is releasably connected to the milk chamber and/or the reservoir. In some embodiments, the first fluid conduit includes a movement element. In some embodiments, the movement element includes a swivel fitting or a rotating fitting. In some embodiments, the movement element includes a ball bearing. In some embodiments, the first fluid conduit is movably connected to the milk chamber. In some embodiments, the first fluid conduit is rotatable with respect to the breast cup.

In some embodiments, the modular breast pump system further includes a second fluid conduit providing fluidic communication between the negative pressure source and/or the positive pressure source and the pressure chamber. In some embodiments, the second fluid conduit is releasably connected to the negative pressure source, the positive pressure source, and/or the pressure chamber.

In some embodiments, the modular breast pump system further includes a control valve.

In some embodiments, the milk chamber further includes an outlet.

In some embodiments, the modular breast pump system further includes a one directional valve configured to allow fluid flow from the milk chamber to the reservoir. In some embodiments, the one directional valve is disposed in the first fluid conduit. In some embodiments, the outlet includes the one directional valve. In some embodiments, the one directional valve is disposed in the diaphragm.

In some embodiments, the breast shield is configured to permit a slow leak to the ambient atmosphere.

In some embodiments, the breast shield includes a wide portion and a nipple tunnel. In some embodiments, the nipple tunnel has a length from about 5 mm to about 75 mm (e.g., about 5 mm to about 25 mm, about 10 mm to about 30 mm, about 25 mm to about 50 mm, about 30 mm to about 60 mm, or about 50 mm to about 75 mm). In some embodiments, the nipple tunnel has a minimum diameter from about 10 mm to about 130 mm (e.g., about 10 mm to about 50 mm, about 25 mm to about 75 mm, about 50 mm to about 100 mm, about 75 mm to about 100 mm, or about 100 mm to about 130 mm). In some embodiments, the nipple tunnel has a maximum diameter from about 10 mm to about 130 mm (e.g., about 10 mm to about 50 mm, about 25 mm to about 75 mm, about 50 mm to about 100 mm, about 75 mm to about 100 mm, or about 100 mm to about 130 mm). In some embodiments, the diameter of the nipple tunnel is between about 20 and about 40 mm.

In some embodiments, the breast shield is funneled.

In some embodiments, the milk chamber includes a volume from about 1 mL to about 150 mL (e.g., about 1 mL to about 10 mL, about 1 mL to about 50 mL, about 10 mL to about 50 mL, about 25 mL to about 50 mL, about 30 mL to about 60 mL, about 50 mL to about 100 mL, about 50 mL to about 150 mL, about 75 mL to about 125 mL or about 100 mL to about 150 mL). In some embodiments, the pressure chamber includes a volume from about 1 mL to about 150 mL (e.g., about 1 mL to about 10 mL, about 1 mL to about 50 mL, about 10 mL to about 50 mL, about 25 mL to about 50 mL, about 30 mL to about 60 mL, about 50 mL to about 100 mL, about 50 mL to about 150 mL, about 75 mL to about 125 mL or about 100 mL to about 150 mL).

In some embodiments, the modular breast pump system further includes a milk chamber wall disposed between the diaphragm and the breast shield, and the diaphragm and the milk chamber wall define the volume of the milk chamber.

In some embodiments, the milk chamber includes an inlet from the breast shield, e.g., wherein the diaphragm releasably contacts the inlet. In some embodiments, the diaphragm releasably seals the inlet.

In some embodiments, the inlet includes a valve. In some embodiments, the valve is mechanical valve, an umbrella valve, a butterfly valve, a disk valve, or a duckbill valve.

In some embodiments, the inlet includes a plurality of orifices. In some embodiments, the plurality of orifices is circumferentially arranged at the nipple tunnel of the breast shield. In some embodiments, each of the plurality of orifices has a diameter from about 0.5 mm to about 10 mm (e.g., about 0.5 mm to about 1 mm, about 0.5 mm to about 2.5 mm, about 1 mm to about 5 mm, about 2 mm to about 7 mm, about 4 mm to about 6 mm, about 5 mm to about 7.5 mm, about 5 mm to about 10 mm, or about 7.5 mm to about 10 mm).

In some embodiments, the breast cup or component thereof, e.g., the breast shield, includes a flexible material. In some embodiments, the breast cup or component thereof, e.g., the breast shield, is polymeric. In some embodiments, the housing includes polyethylene terephthalate (PET), polypropylene (PP), polytetrafluoroethylene (PTFE) (e.g., Teflon), polyethylene, high-density polyethylene (HDPE), low-density polyethylene (LDPE), copolyester (e.g., Tritan EX401, polycarbonate (PC), nylon, polyvinylidene fluoride (PVDF), silicone, polyester, cotton, linen, satin, organdy, rayon, taffeta, broad cloth, poplin, velour, gauze, canvas, shirting, muslin, tweed, georgette, crepe, wool, twill, gabardine, denim, or drill.

In some embodiments, the breast cup or component thereof, e.g., the breast shield, include ridges, bumps or dimples. In some embodiments, the breast cup includes a toroidal ridge.

In some embodiments, the modular breast pump system further includes two breast cups, wherein the two breast cups each include a housing, a breast shield, and a diaphragm as described herein.

In some embodiments, the diaphragm includes a flexible material. In some embodiments, the diaphragm includes a polymeric material.

In some embodiments, the diaphragm includes a material having a Shore hardness from A10 to A80. In some embodiments, the diaphragm includes a material having a shore hardness from A10 to A80 and a second Shore hardness from A10 to A80 or being rigid, wherein the material includes the second Shore hardness or is rigid where the diaphragm contacts the inlet. In some embodiments, the diaphragm includes a first material, e.g., having a Shore hardness from A10 to A80, and a second material, e.g., having a different shore hardness from A10 to A80 or being rigid. In some embodiments, either the first or second material may have a Shore hardness from D10 to D80. The second material may be more rigid than the first material and contact the inlet.

In some embodiments, the diaphragm includes a thickness from about 1 mm to about 10 mm (e.g., about 1 mm to about 2 mm, about 1 mm to about 3 mm, about 1 mm to about 5 mm, about 2 mm to about 7 mm, about 4 mm to about 6 mm, about 5 mm to about 7.5 mm, about 5 mm to about 10 mm, or about 7.5 mm to about 10 mm). In some embodiments, the diaphragm includes a first thickness from about 1 mm to about 10 mm (e.g., about 1 mm to about 2 mm, about 1 mm to about 3 mm, about 1 mm to about 5 mm, about 2 mm to about 7 mm, about 4 mm to about 6 mm, about 5 mm to about 7.5 mm, about 5 mm to about 10 mm, or about 7.5 mm to about 10 mm), and a second thickness from about 1 mm to about 10 mm (e.g., about 1 mm to about 2 mm, about 1 mm to about 3 mm, about 1 mm to about 5 mm, about 2 mm to about 7 mm, about 4 mm to about 6 mm, about 5 mm to about 7.5 mm, about 5 mm to about 10 mm, or about 7.5 mm to about 10 mm), wherein the diaphragm includes the second thickness where the diaphragm contacts the inlet. The thickness of the diaphragm may be non-uniform. For example, portions at the edge or contacting the inlet may be thicker or thinner than other portions.

In some embodiments, the diaphragm includes a plurality of layers. In some embodiments, the diaphragm includes a plurality of layers wherein the diaphragm contacts the inlet.

In some embodiments, the diaphragm is shaped to conform to the shape of breast shield or the milk chamber wall. In some embodiments, the diaphragm includes a curved shape.

In some embodiments, the pressure chamber further includes a valve to the ambient atmosphere. In some embodiments, a volume of the pressure chamber may be set with the valve. In some embodiments, the valve is an inflation valve.

All or portions of the breast cup may be clear, e.g., to allow for visualization of the nipple during placement of the breast cup. A portion, e.g., a portion of the diaphragm, may also include a lens to magnify the nipple for alignment. Breast cups with clear (e.g., transparent) portions may include a flap or other opaque covering to hide the clear portions after alignment.

The present disclosure provides a method of breast pumping including providing the modular breast pump system of the present disclosure, contacting a human breast of a subject with a breast cup; and pumping milk from the human breast by alternating reducing and increasing pressure in the pressure chamber.

In some embodiments, the modular breast pump system may be used when the subject is moving, resting, sleeping, or traveling.

In some embodiments, the method further includes connecting the second fluid conduit to the breast cup and negative pressure source and/or positive pressure source. In some embodiments, the method further includes connecting the first fluid conduit to the breast cup and the reservoir. In some embodiments, the method further includes connecting the second fluid conduit to the negative pressure source and the pressure chamber. In some embodiments, the method further includes connecting the third fluid conduit to the control valve, the breast cup, and/or the second fluid conduit. In some embodiments, the method further includes connecting the fourth fluid conduit to the breast cup and the reservoir.

In some embodiments, the method further includes contacting the human breast with the wide portion of the breast cup. In some embodiments, the method further includes contacting the human breast with the wide portion of the breast shield. In some embodiments, a nipple of the breast is placed in the nipple tunnel of the breast cup. In some embodiments, the method further includes contacting two human breasts with the two breast cups.

In some embodiments, the method further includes applying negative pressure to the pressure chamber with the negative pressure source. In some embodiments, the method further includes deforming the diaphragm away from the breast shield. In some embodiments, the method further includes deforming the diaphragm away from the inlet. In some embodiments, the milk chamber comprises an inlet, and the diaphragm pulls away from the inlet to provide fluidic communication between the milk chamber and the breast shield when pressure in the pressure chamber is reduced. In some embodiments, the method further includes unsealing the inlet. In some embodiments, the method further includes expanding the milk chamber. In some embodiments, the method further includes contracting the pressure chamber. In some embodiments, the method further includes providing negative pressure to elongate the nipple. In some embodiments, the method further includes drawing milk from the nipple. In some embodiments, the method further includes drawing milk from the nipple tunnel into the milk chamber. In some embodiments, the method further includes drawing milk through the inlet into the milk chamber.

In some embodiments, the method further includes opening the control valve and depressurizing the pressure chamber. In some embodiments, the method further includes returning the diaphragm to a nominal position. In some embodiments, the method further includes closing the control valve.

In some embodiments, the method further includes applying positive pressure to the pressure chamber with the positive pressure source. In some embodiments, the method further includes allowing the nipple to contract. In some embodiments, the method further includes deforming the diaphragm towards the breast shield. In some embodiments, the method includes allowing the diaphragm to return to a nominal position. In some embodiments, the method further includes sealing the inlet with the diaphragm. In some embodiments, the diaphragm seals the inlet when pressure in the pressure chamber increases. In some embodiments, the method further includes sealing orifices in the inlet with the diaphragm. In some embodiments, the method further includes contracting the milk chamber. In some embodiments, the method further includes expanding the pressure chamber. In some embodiments, the method further includes transporting milk from the milk chamber through the first fluid conduit to the reservoir. In some embodiments, the method further includes transporting milk through the outlet. In some embodiments, the method further includes transporting milk through the one directional valve.

In some embodiments, the method further includes applying negative pressure to a fluid conduit attached to an outlet of the milk chamber. In some embodiments, the negative pressure is applied via a peristaltic pump or a vacuum pump. In some embodiments, the fluid conduit comprises an air inlet.

In some embodiments, the method further includes waiting at least 30 minutes after the pumping step and repeating the pumping step to a provide a first and second breast pumping session. In some embodiments, the method further includes pumping milk from the human breast twice, wherein there is at least about 30 minutes (e.g., about 45 minutes, about 60 minutes, about 75 minutes, about 90 minutes, about 120 minutes, about 150 minutes, about 180 minutes, about 210 minutes, or about 240 minutes) between a first breast pumping session to a second breast pumping session.

In some embodiments, milk from the first breast pumping session is transported to a first reservoir, the first reservoir is removed, and milk from the second breast pumping session is transported to a second reservoir. In some embodiments, milk from the first pumping session is transported into the first reservoir, the first reservoir is removed and then replaced, and milk from the second breast pumping session is transported to the replaced first reservoir. In some embodiments, following the first pumping session, milk from the first reservoir is fed to a child, and the first reservoir is replaced prior to the start of the second pumping session. In some embodiments, following the first pumping session, milk from the first reservoir is disposed of, and the first reservoir is replaced prior to the start of the second pumping session.

In some embodiments, milk from the first breast pumping session is transported to a first reservoir, and milk from the second pumping session is transported to a second reservoir, e.g., via a manifold. In some embodiments, the manifold directs milk to the first reservoir and the second reservoir by moving a movable member to provide sequential fluidic communication between an inlet to the manifold and the first and second reservoirs.

In some embodiments, milk from the first breast pumping session is transported to a first reservoir fluidically connected to the portable module, and milk from the second breast pumping session is transported to a second reservoir disposed of in the base module. In some embodiments, the manifold transports milk to a plurality of reservoirs disposed of in the base module. In some embodiments, the method further includes connecting the portable module and the base module after the first pumping session is complete, and prior to the start of the second pumping session.

In some embodiments, the method further includes pumping milk from the human breast to induce milk from a first breast pumping session to flow from a nipple to the manifold via the breast cup; providing fluidic communication between the inlet of the manifold and a first reservoir to allow milk from the first breast pumping session to flow into the first reservoir; removing fluidic communication between the inlet of the manifold and the first fluid reservoir; pumping the human breast to induce milk from a second breast pumping session to flow from the nipple to the manifold via the breast cup; and providing fluidic communication between the inlet of the manifold and a second reservoir to allow milk from the second breast pumping session to flow into the second reservoir. In some embodiments, the method further includes from 30 minutes to 240 minutes (about 30 minutes to 60 minutes, about 30 minutes to 90 minutes, about 60 minutes to about 180 minutes, about 60 minutes to about 120 minutes, about 90 minutes to 120 minutes, or about 120 minutes to 240 minutes) between the first and second sessions.

In some embodiments, the method further includes removing fluidic communication between the inlet of the manifold and the second fluid reservoir; providing fluidic communication between the inlet of the manifold and a third reservoir; and pumping the human breast to induce milk from a third breast pumping session to flow from the nipple to the manifold via the breast cup. In some embodiments, the method further includes from 30 minutes to 240 minutes (about 30 minutes to 60 minutes, about 30 minutes to 90 minutes, about 60 minutes to about 180 minutes, about 60 minutes to about 120 minutes, about 90 minutes to 120 minutes, or about 120 minutes to 240 minutes) between step the second and third sessions.

In some embodiments, the method further includes providing fluidic communication between (i) the inlet of the manifold and (ii) the first reservoir, the second reservoir, or the third fluid reservoir by moving the movable, e.g., rotating, member. In some embodiments, the method further includes removing fluidic communication between (i) the inlet of the manifold and (ii) the first reservoir, the second reservoir, or the third fluid reservoir by moving, e.g., rotating, the movable, e.g., rotating, member.

In some embodiments, the method further includes providing fluidic communication between (i) the inlet of the manifold and (ii) the first reservoir, the second reservoir, or the third fluid reservoir by opening the valve to the inlet of the first reservoir, the second reservoir, or the third fluid reservoir. In some embodiments, the method further includes removing fluidic communication between (i) the inlet of the manifold and (ii) the first reservoir, the second reservoir, or the third fluid reservoir by closing the valve to the inlet of the first reservoir, the second reservoir, or the third fluid reservoir.

In some embodiments, the method further includes providing fluidic communication between (i) the inlet of the manifold and (ii) the first reservoir, the second reservoir, or the third fluid reservoir by moving the arm. In some embodiments, the method further includes removing fluidic communication between (i) the inlet of the manifold and (ii) the first reservoir, the second reservoir, or the third fluid reservoir by moving the arm. In some embodiments, the method further includes moving the arm with the drive unit, wherein the drive unit is the stepper motor.

In some embodiments, the method further includes taking a weight, a temperature, a pressure, an optical, a flow rate, a pH, or a viscosity measurement of the milk with the sensor. In some embodiments, the method further includes alerting a user of the temperature, pressure, optical, flow rate, volume, pH, or viscosity measurement. In some embodiments, the method further includes changing at least one of the first pressure or second pressure based on the temperature, pressure, or flow rate measurement. In some embodiments, the method further includes measuring the weight of a reservoir containing the pumped milk.

In some embodiments, the method further includes securing the breast cup to the garment and donning the garment. In some embodiments, the method further includes donning the garment and securing the breast cup to the garment.

In some embodiments, the method further includes securing the portable module to the subject with a strap or a fastener. In some embodiments, the method further includes portable module traveling in time with the subject's body.

In some embodiments, the method further includes removing the portable module from connection with the base module. In some embodiments, the method further includes holding the portable module by a handle and moving the portable module from a first location to a second location. In some embodiments, the method includes a subject contacting the sides or bottom of the portable module and holding the portable module. In some embodiments, the method further includes mating the portable module and the base module.

In some embodiments, the method further includes pumping milk from the human breast at least twice without removing the breast cup from the human breast.

The present disclosure provides a method of cleaning a modular breast pump system: (i) providing the modular breast pump system of the present disclosure, (ii) providing a cleaning fluid to the breast cup; and (iii) pumping the cleaning fluid from the breast cup to the reservoir.

In some embodiments, a subject including the human breast is moving, reclining, resting, sleeping, or traveling.

The present disclosure further provides a kit comprising a base module, an portable module, a reservoir, optionally one and/or two breast cups, a manifold and a plurality of reservoirs (e.g., 2 to 3, 2 to 4, 2 to 5, 2 to 6, 2 to 8, 2 to 10, 3 to 4, 3 to 5, 3 to 6, 4 to 5, 4 to 6, 4 to 8, 4 to 10, 5 to 10, 2, 3, 4, 5, 6, 7, 8, 9, 10, 11, 12, 13, 14, 15, 16, 17, 18, 19, or 20).

### Definitions

To facilitate the understanding of this disclosure, a number of terms are defined below and throughout the disclosure. Unless otherwise defined, all technical and scientific terms used herein have the same meaning as commonly understood by one of ordinary skill in the art to which this disclosure belongs. The terminology herein is used to describe specific embodiments of the disclosure, but their usage does not limit the disclosure, except as outlined in the claims.

Terms such as “a”, “an,” and “the” are not intended to refer to only a singular entity but include the general class of which a specific example may be used for illustration.

The term “fluidically connected,” as used herein, refers to a direct connection between at least two device elements, e.g., a channel, reservoir, etc., that allows for fluid to move between such device elements without passing through an intervening element.

The term “fluidically disconnected,” as used herein refers to the absence of a connection in which fluid, e.g., a liquid, can flow between elements in a fluid path.

The term “food contact substance,” as used herein, refers to a substance or material that is intended for use as a component in manufacturing, packing, packaging, transporting, or holding food in which such use is not intended to have any technical effect in such food.

The term “fluidic communication,” as used herein, refers to a connection between at least two device elements, e.g., a channel, reservoir, etc., that allows for fluid to move between such device elements with or without passing through one or more intervening device elements.

The term “valve,” as used herein, refers to an element which regulates, directs or controls the flow of a fluid by opening, closing, or partially obstructing a fluid pathway.

The term “portable,” as used herein, refers to an object that a subject can transport the object either in a pocket area, strapped to the body, or otherwise on their person.

The term “positive pressure,” as used herein, refers to an application of pressure from a pressure source (e.g., a pump) that tends to increase a pressure within a given component or space.

The term “negative pressure,” as used herein, refers to an application of pressure from a pressure source (e.g., a pump) that tends to decrease a pressure within a given component or space.

### BRIEF DESCRIPTION OF THE DRAWINGS

The following detailed description of the embodiments of the disclosure will be better understood when read in conjunction with the appended drawings. For the purpose of illustrating the disclosure, there are shown in the drawing embodiments, which are presently exemplified. It should be understood, however, that the disclosure is not limited to the precise arrangement and instrumentalities of the embodiments shown in the drawings.

FIG. 1 shows a schematic of a breast cup including a wide portion and a nipple tunnel, according to an example embodiment.

FIG. 2 shows a reservoir enclosure including a reservoir containing milk and a one directional valve, according to an example embodiment.

FIG. 3A and FIG. 3B show schematic drawings of a breast pump system, according to an example embodiment.

FIG. 4 shows a schematic drawing of breast cup, including housing, diaphragm, breast shield, pressure chamber, milk chamber, outlet, and sealing point, according to an example embodiment.

FIG. 5 shows a schematic drawing of breast cup, including housing, diaphragm, breast shield, pressure chamber, milk chamber, outlet, and valve, according to an example embodiment.

FIG. 6 shows a schematic drawing of breast cup, including housing, diaphragm, breast shield, pressure chamber, milk chamber, outlet, sealing point, and orifices, according to an example embodiment.

FIG. 7 shows a schematic drawing of breast cup, including housing, diaphragm, breast shield, pressure chamber, milk chamber, outlet, sealing point, and milk chamber wall, according to an example embodiment.

FIG. 8A and FIG. 8B show schematic drawings of breast shield having a protrusion on the inner surface of breast shield, wherein the inner surface of breast shield is configured to contact the breast of a user, according to an example embodiment. FIG. 8A shows a schematic drawing of breast shield having a toroidal ridge on the inner surface of breast shield, according to an example embodiment. FIG. 8B shows a schematic drawing of breast shield having a curved toroidal ridge on the inner surface of breast shield, according to an example embodiment.

FIG. 9 shows a schematic drawing of a milk distribution system including (i) a plurality of reservoirs and (ii) a manifold having an inlet, at least one outlet, and an integral drive unit, wherein the manifold is configured to rotate to provide fluidic communication between an outlet of the manifold and a reservoir, according to an example embodiment.

FIG. 10 shows a schematic drawing of a milk distribution system including (i) a plurality of reservoirs, (ii) a drive unit, and (iii) a manifold having an inlet and at least one outlet, wherein the drive unit is configured to rotate the manifold to provide fluidic communication between an outlet of the manifold and a reservoir, according to an example embodiment.

FIG. 11 shows a schematic drawing of a milk distribution system including (i) a plurality of reservoirs and (ii) a manifold having an inlet and at least out outlet, wherein each of the reservoirs includes a valve, in particular, a solenoid valve, wherein the solenoid valve is configured to provide fluidic communication between an outlet of the manifold and a reservoir, according to an example embodiment.

FIG. 12 shows a schematic drawing of a milk distribution system including (i) a plurality of reservoirs, (ii) a pressure source, (iii) and a manifold having an inlet and at least one outlet, wherein the pressure source is configured to provide fluidic communication between an outlet of the manifold and a reservoir, according to an example embodiment.

FIGS. 13A-13D show views of a breast cup including housing, diaphragm, breast shield, pressure chamber, milk chamber, milk chamber wall, ports, and a one directional valve, according to an example embodiment.

FIGS. 14A-14B show a milk distribution system including a manifold with four arms, an inlet, outlet, alignment plate, weighing plate, vertical body, gasket, drive dog, stationary base, and a stepper motor, according to an example embodiment.

FIG. 15 shows a schematic of a milk distribution system with a flush line and negative pressure source, according to an example embodiment.

FIG. 16 shows a schematic of a modular breast pump system, including an portable module and a base module, according to an example embodiment.

FIG. 17 shows a perspective view of a portable module for use with the module breast pump system of FIG. 16, according to an example embodiment.

FIG. 18 shows an exploded view of the portable module of FIG. 17, according to an example embodiment.

FIG. 19 shows a side view of the portable module of FIG. 17, according to an example embodiment.

FIG. 20 shows a section view of the portable module of FIG. 19, taken along section line A-A, according to an example embodiment.

FIG. 21 shows a section view of the portable module of FIG. 19, taken along section line B-B, according to an example embodiment.

FIG. 22 shows a perspective view of the portable module of FIG. 17, shown with a cap of the portable module removed, according to an example embodiment.

FIG. 23 shows another perspective view of the portable module of FIG. 17, shown with the cap of the portable module removed, according to an example embodiment.

FIG. 24 shows a top view of the portable module of FIG. 17, according to an example embodiment.

FIG. 25 shows a top view of the portable module of FIG. 17, shown with a cap of the portable module removed, according to an example embodiment.

FIG. 26 shows a top view of the portable module of FIG. 17, shown with a cap and an end cover of the portable module removed, according to an example embodiment.

FIG. 27 shows a perspective view of a base module for use with the module breast pump system of FIG. 16, according to an example embodiment.

FIG. 28 shows a perspective view of the base module of FIG. 27, shown with a lid of the base module removed, according to an example embodiment.

FIG. 29 shows a perspective view of the base module of FIG. 27, shown with the lid of the base module removed and a removable pumping unit detached from the base module, according to an example embodiment.

FIG. 30 shows a top view of the base module of FIG. 27, shown with the lid of the base module removed, according to an example embodiment.

FIG. 31 shows a top view of the base module of FIG. 27, shown with a lid of the base module removed and the removable pumping unit detached from the base module, according to an example embodiment.

FIG. 32 shows a rear view of a removable pumping unit, according to an example embodiment.

FIG. 33 shows a front view of the base module of FIG. 27, shown with the removable pumping unit removed, according to an example embodiment.

FIG. 34 shows a front view of the base module of FIG. 27, according to an example embodiment.

FIG. 35 shows a section view of the base module of FIG. 34, take along line B-B, according to an example embodiment.

FIG. 36 shows a front view of the base module of FIG. 27, according to an example embodiment.

FIG. 37 shows a section view of the base module of FIG. 36, take along line H-H, according to an example embodiment.

FIG. 38 shows a front view of the base module of FIG. 27, according to an example embodiment.

FIG. 39 shows a section view of the base module of FIG. 38, take along line E-E, according to an example embodiment.

FIG. 40 shows a rear view of the base module of FIG. 27, according to an example embodiment.

FIG. 41 shows a section view of the base module of FIG. 40, take along line E-E, according to an example embodiment.

### DETAILED DESCRIPTION

The present disclosure provides improved modular breast pump systems, components thereof, e.g., a base module and an portable module, and methods of use thereof. The present modular breast pump systems are particularly advantageous for navigating daily activities, discreet public pumping and accessing pumped milk without removal of the breast cups, moving, resting, reclining, sleeping, or traveling.

The present disclosure further provides improved milk distribution systems and methods of use thereof. The present milk distribution systems are particularly advantageous for use in a series of breast pumping sessions without removal of a breast cup and/or without replacement of a reservoir in the breast pump system.

### Modular Breast Pump System

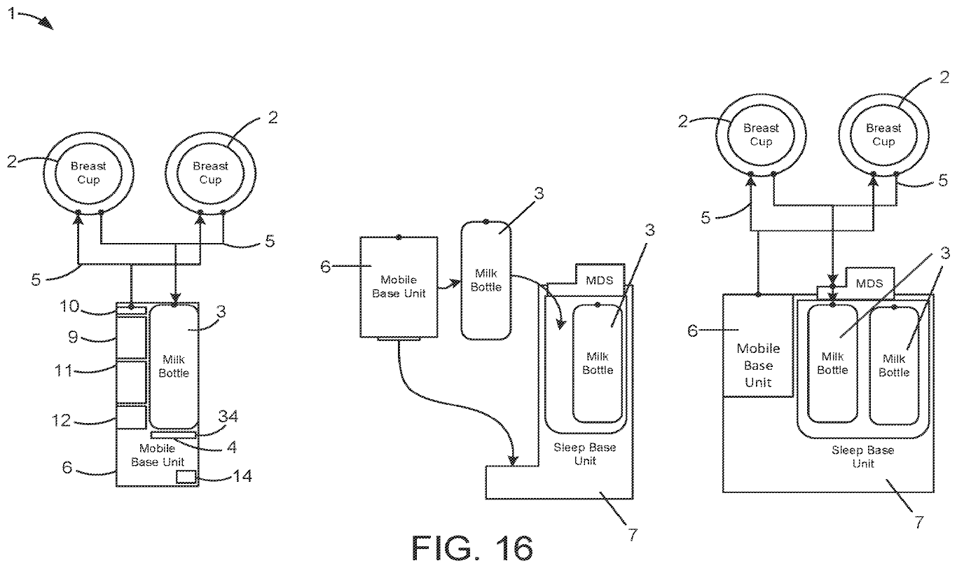

The present disclosure provides an improved modular breast pump system 1. The modular breast pump system includes an accessory or portable module 6 and a main or base module 7. The modules may be mated together or separated. For example, when at home, the modules are mated, but the portable module 6 can be decoupled from the base module 7 for breast pumping on the go. The modular breast pump system 1 is advantageous in that it allows for maximum mobility during pumping without the need for an outlet; provides for discreet public pumping without necessitating removal of the breast cup to remove milk after pumping; comfortably allows for pumping in any position, such as lying reclined, flat, or on the side; allows for pumping without assembly at the time of use; allows for a plurality of breast pumping sessions without the removal of the breast cup 2; decreases the likelihood of waking up a partner while breast pumping; and/or allows for the collection and storage of milk for extended periods of time, e.g., while the mother is traveling outside the home, resting, or sleeping An exemplary system is shown in FIG. 16.

As seen in FIG. 16, the modular breast pump system 1 may include two breast cups 2 that can be in fluidic communication with a pressure source disposed of in the portable module 6. The breast cups may also be in fluidic communication with a reservoir disposed of in the portable module 6 and/or the base module 7. The modular breast pumping system may be utilized with either the portable module 6 alone, or with the portable module 6 mated to the base module 7.

The modular breast pump system 1 may include at least one breast cup 2 in fluidic communication with a pressure source 9 and a reservoir 3.

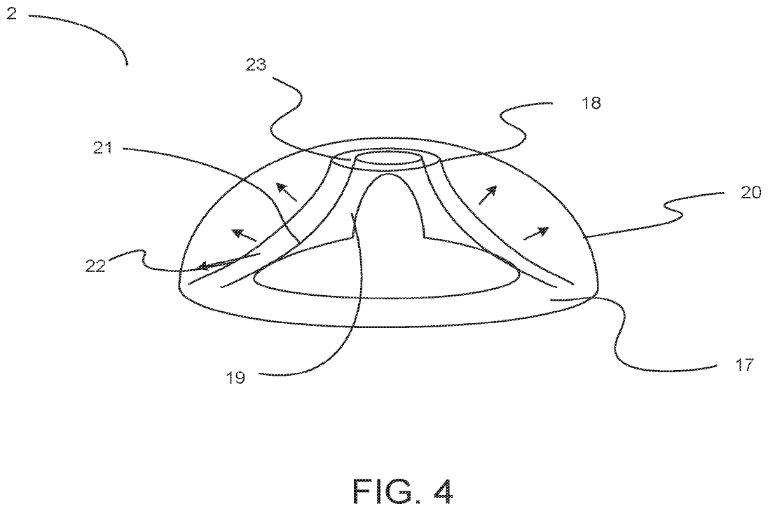

As shown in, e.g., FIG. 4, the modular breast pump system 1 can include a breast cup 2 having a housing including an upper housing 17 and a lower housing or milk chamber wall 26, a breast shield 19, and a diaphragm 18 disposed between the upper housing 17 and the breast shield 19 (and also between the upper housing 17 and the lower housing 26). The diaphragm 18 can separate the pressure chamber 20 from the milk chamber 21. The diaphragm provides numerous advantages including improved breast pumping in reclined positions. A pressure source 9 (e.g., a negative pressure source and/or a positive pressure source) can be in fluidic communication with the pressure chamber 20, and the reservoir 3 can be in fluidic communication with the milk chamber 21.

The modular breast pump system 1 can include a food contact substance, e.g., for surfaces that contact milk and the breast. Alternatively, or in addition, the breast pump system 1 can include an infant grade material.

The modular breast pump system 1, including any element therein, can include a polymer (e.g., polyvinyl chloride (PVC), polyethylene terephthalate (PET), polypropylene (PP), polytetrafluoroethylene (PTFE), polyethylene, high-density polyethylene (HDPE), low-density polyethylene (LDPE), copolyester (e.g., Tritan EX401, polystyrene, a thermoplastic elastomer, thermoplastic polyurethane, nylon, polyvinylidene fluoride (PVDF), silicone, or polycarbonate (PC)). For example, the reservoir 3, the reservoir enclosure 4, the fluid conduit 5, or the breast cup 2, including the housing (e.g., the upper housing 17 and the lower housing or milk chamber wall 26), breast shield 19, and diaphragm 18 can include a polymer.

In some embodiments, modular breast pump system 1 can include a fabric (e.g., polyester, cotton, linen, satin, organdy, rayon, taffeta, broad cloth, poplin, velour, gauze, canvas, shirting, muslin, tweed, georgette, crepe, wool, twill, gabardine, denim, or drill). For example, the reservoir 3, the reservoir enclosure 4, or the breast cup 2, including the housing (e.g., the upper housing 17 and the lower housing or milk chamber wall 26) and the breast shield 19, can include a fabric.

In some embodiments, the modular breast pump system 1 includes a foam, e.g., an open cell foam. For example, the reservoir 3, the reservoir enclosure 4, or the breast cup 2, including the housing (e.g., the upper housing 17 and the lower housing or milk chamber wall 26) and the breast shield 19, can include a foam.

### Modules

As shown in FIG. 16, the base module 7 and the portable module 6 mate together. The combined modules may include a negative pressure source, a drive unit, a power source, and a breast milk distribution system. In some embodiments, one of the modules further includes a positive pressure source. In some embodiments, the positive pressure source is the same as the negative pressure source. In some embodiments, the combined modules include a plurality of pressure sources, power sources, drive units, valves, sensors, and reservoirs.

In some embodiments, the modules are connected physically to make a single unit, such that the connected unit may be safely carried. The two modules may be aligned with magnets and/or shaped to snap or otherwise fit together. Physical connections can be made by any suitable manner, including magnets, clips, zippers, snaps, latches, etc. In some embodiments, the modules are connected electrically to make a single unit. For example, the base module 7 may power the portable module 6 and/or recharge a battery in the portable module 6 when the modules are mated. Any suitable electrical connection may be employed, e.g., plug and socket. In some embodiments, the modules are fluidically connected with respect to the pressure source.

In some embodiments, a first fluidic conduit fluidically connects a pressure source in the portable module 6 to the breast cup. In some embodiments, a second fluidic conduit fluidically connects the breast cup to a reservoir releasably connected to the portable module 6. In some embodiments, the second fluidic conduit fluidically connects the breast cup to a reservoir disposed in the base module 7. In some embodiments, the second fluidic conduit fluidically connects the breast cup to a manifold in a breast milk distribution system in the base module 7.

The modules may include any polymer (e.g., PVC, PET, PP, PTFE, polyethylene, HDPE, LDPE, copolyester (e.g., Tritan EX401, polystyrene, a thermoplastic elastomer, a thermoplastic polyurethane, nylon, PVDF, or PC) or other suitable material, e.g., metal or ceramic. The modules may include outer components including a base, a body, and a cover that can releasably or non-releasably be secured together. The modules may be injection molded.

### Base Module

The base module 7 may include various components including a breast milk distribution system. In some embodiments, the base module 7 further includes a pressure source, a control valve, a reservoir enclosure, and a power source. In some embodiments, the base module 7 further includes a plurality of reservoirs, valves, and/or sensors.

In some embodiments, the base module 7 may be configured such that a breast pumping session may occur with the base module 7 alone. In some embodiments, the base module 7 may be configured such that a breast pumping system may only occur when the portable module 6 and base module 7 are mated. In one embodiment, a pressure source in the portable module 6 is used to induce lactation, while collection of milk occurs in the base module 7. That is, in some embodiments, the base module 7 does not include a pressure source for inducing lactation. In some embodiments, the base module 7 includes a pressure source for transporting milk after lactation.

In some embodiments, the base module 7 may include a power source, such that when mated to the portable module 6, the base module 7 provides any power required for operation. In some embodiments, the base module 7 may further charge a power source of the portable module 6 when mated.

In some embodiments, the base module 7 includes a reservoir enclosure 4. In some embodiments, the reservoir enclosure in the base module 7 includes a cooling element and/or insulation. In some embodiments, the reservoir in the base module 7 includes space for a plurality of reservoirs. In some embodiments, the base module 7 includes a plurality of reservoirs. In some embodiments the reservoir enclosure in the base module 7 includes one or more sensors including one or more of a weight sensor, a load cell, a temperature sensor, a pressure sensor, an optical sensor, a flow sensor, a volume sensor, a pH sensor, and a viscosity sensor.

The base module 7 may include any polymer (e.g., PVC, PET, PP, PTFE, polyethylene, HDPE, LDPE, copolyester (e.g., Tritan EX401, polystyrene, a thermoplastic elastomer, a thermoplastic polyurethane, nylon, PVDF, or PC) or other suitable material, e.g., metal or ceramic. The base module 7 may include outer components including a base, a body, and a cover that can releasably or non-releasably be secured together. The base module 7 may be injection molded.

### Portable Module

The portable module 6 may include various components including a pressure source 9 (e.g., a negative pressure source and/or a positive pressure source), a control valve 10, a drive unit 11, and a power source 12. In some embodiments, the portable module 6 includes a second power source 16 and/or one or more control valves 10. In some embodiments, the negative pressure source is the same as the positive pressure source. The portable module 6 may be portable such that a subject can move the portable module 6 from one location to another location a plurality of times before, during, and/or after a pumping session. For example, the user may decouple the portable module 6 from the base module 7 for breast pumping on the go.

In some embodiments, the portable module 6 includes a reservoir enclosure 4. In some embodiments, the portable module 6 includes a lid, and the reservoir enclosure may be accessible through the lid. In some embodiments, the portable module 6 does not include a lid to access the reservoir enclosure. In some embodiments, a reservoir is releasably secured in the reservoir enclosure disposed in the portable module 6 such that the reservoir does not unintentionally separate from the portable module 6. In some embodiments, the reservoir enclosure in the portable module 6 includes a cooling element and/or insulation. In some embodiments the reservoir enclosure in portable module 6 includes one or more sensors including one or more of a weight sensor, a load cell, a temperature sensor, a pressure sensor, an optical sensor, a flow sensor, a volume sensor, a pH sensor, and a viscosity sensor. In some embodiments, the reservoir enclosure in the portable module 6 holds only a single reservoir.

In some embodiments, the portable module 6 may include a reservoir that is releasably connected to the portable module 6 in the absence of a reservoir enclosure. For example, the reservoir may be connected to the portable module 6 via an opening in the reservoir where milk may enter the reservoir. In some embodiments, the reservoir may be fluidically connected to the portable module 6. In some embodiments, the reservoir may include internal and/or external threads such that it may be releasably attached to the portable module 6. In some embodiments, the reservoir may be releasably attached to the portable module 6 via a latch, snap fit, or other mechanism.

The portable module 6 may include any polymer (e.g., PVC, PET, PP, PTFE, polyethylene, HDPE, LDPE, copolyester (e.g., Tritan EX401, polystyrene, a thermoplastic elastomer, a thermoplastic polyurethane, nylon, PVDF, or PC) or other suitable material, e.g., metal or ceramic. The portable module 6 may include outer components including a base, a body, and a cover that can releasably or non-releasably be secured together. The portable module 6 may be injection molded.

In some embodiments, the portable module 6 includes a weight of between about 1 pound and about 20 pounds (e.g., about 1 to 2 pounds, about 1 to 3 pounds, about 1 to 4 pounds, about 1 to 5 pounds, about 1 to 6 pounds, about 1 to 7 pounds, about 1 to 8 pounds, about 1 to 9 pounds, about 1 to 10 pounds, about 1 to 11 pounds, about 1 to 12 pounds, about 1 to 13 pounds, about 1 to 14 pounds, about 1 to 15 pounds, about 1 to 16 pounds, about 1 to 17 pounds, about 1 to 18 pounds, about 1 to 19 pounds, about 1 to 20 pounds, about 2 to 3 pounds, about 2 to 4 pounds, about 2 to 5 pounds, about 2 to 6 pounds, about 2 to 7 pounds, about 2 to 8 pounds, about 2 to 9 pounds, about 2 to 10 pounds, about 2 to 11 pounds, about 2 to 12 pounds, about 2 to 13 pounds, about 2 to 14 pounds, about 2 to 15 pounds, about 2 to 16 pounds, about 2 to 17 pounds, about 2 to 18 pounds, about 2 to 19 pounds, about 2 to 20 pounds, about 3 to 4 pounds, about 3 to 5 pounds, about 3 to 6 pounds, about 3 to 7 pounds, about 3 to 8 pounds, about 3 to 9 pounds, about 3 to 10 pounds, about 3 to 11 pounds, about 3 to 12 pounds, about 3 to 13 pounds, about 3 to 14 pounds, about 3 to 15 pounds, about 3 to 16 pounds, about 3 to 17 pounds, about 3 to 18 pounds, about 3 to 19 pounds, about 3 to 20 pounds, about 4 to 5 pounds, about 4 to 6 pounds, about 4 to 7 pounds, about 4 to 8 pounds, about 4 to 9 pounds, about 4 to 10 pounds, about 4 to 11 pounds, about 4 to 12 pounds, about 4 to 13 pounds, about 4 to 14 pounds, about 4 to 15 pounds, about 4 to 16 pounds, about 4 to 17 pounds, about 4 to 18 pounds, about 4 to 19 pounds, about 4 to 20 pounds, about 5 to 6 pounds, about 5 to 7 pounds, about 5 to 8 pounds, about 5 to 9 pounds, about 5 to 10 pounds, about 5 to 11 pounds, about 5 to 12 pounds, about 5 to 13 pounds, about 5 to 14 pounds, about 5 to 15 pounds, about 5 to 16 pounds, about 5 to 17 pounds, about 5 to 18 pounds, about 5 to 19 pounds, about 5 to 20 pounds, about 6 to 7 pounds, about 6 to 8 pounds, about 6 to 9 pounds, about 6 to 10 pounds, about 6 to 11 pounds, about 6 to 12 pounds, about 6 to 13 pounds, about 6 to 14 pounds, about 6 to 15 pounds, about 6 to 16 pounds, about 6 to 17 pounds, about 6 to 18 pounds, about 6 to 19 pounds, about 6 to 20 pounds, about 7 to 8 pounds, about 7 to 9 pounds, about 7 to 10 pounds, about 7 to 11 pounds, about 7 to 12 pounds, about 7 to 13 pounds, about 7 to 14 pounds, about 7 to 15 pounds, about 7 to 16 pounds, about 7 to 17 pounds, about 7 to 18 pounds, about 7 to 19 pounds, about 7 to 20 pounds, about 8 to 9 pounds, about 8 to 10 pounds, about 8 to 11 pounds, about 8 to 12 pounds, about 8 to 13 pounds, about 8 to 14 pounds, about 8 to 15 pounds, about 8 to 16 pounds, about 8 to 17 pounds, about 8 to 18 pounds, about 8 to 19 pounds, about 8 to 20 pounds, about 9 to 10 pounds, about 9 to 11 pounds, about 9 to 12 pounds, about 9 to 13 pounds, about 9 to 14 pounds, about 9 to 15 pounds, about 9 to 16 pounds, about 9 to 17 pounds, about 9 to 18 pounds, about 9 to 19 pounds, about 9 to 20 pounds, about 10 to 11 pounds, about 10 to 12 pounds, about 10 to 13 pounds, about 10 to 14 pounds, about 10 to 15 pounds, about 10 to 16 pounds, about 10 to 17 pounds, about 10 to 18 pounds, about 10 to 19 pounds, about 10 to 20 pounds, about 11 to 12 pounds, about 11 to 13 pounds, about 11 to 14 pounds, about 11 to 15 pounds, about 11 to 16 pounds, about 11 to 17 pounds, about 11 to 18 pounds, about 11 to 19 pounds, about 11 to 20 pounds, about 12 to 13 pounds, about 12 to 14 pounds, about 12 to 15 pounds, about 12 to 16 pounds, about 12 to 17 pounds, about 12 to 18 pounds, about 12 to 19 pounds, about 12 to 20 pounds, about 13 to 14 pounds, about 13 to 15 pounds, about 13 to 16 pounds, about 13 to 17 pounds, about 13 to 18 pounds, about 13 to 19 pounds, about 13 to 20 pounds, about 14 to 15 pounds, about 14 to 16 pounds, about 14 to 17 pounds, about 14 to 18 pounds, about 14 to 19 pounds, about 14 to 20 pounds, about 15 to 16 pounds, about 15 to 17 pounds, about 15 to 18 pounds, about 15 to 19 pounds, about 15 to 20 pounds, about 16 to 17 pounds, about 16 to 18 pounds, about 16 to 19 pounds, about 16 to 20 pounds, about 17 to 18 pounds, about 17 to 19 pounds, about 17 to 20 pounds, about 18 to 19 pounds, about 18 to 20 pounds, about 19 to 20 pounds, about 1 pound, about 2 pounds, about 3 pounds, about 4 pounds, about 5 pounds, about 6 pounds, about 7 pounds, about 8 pounds, about 9 pounds, about 10 pounds, about 11 pounds, about 12 pounds, about 13 pounds, about 14 pounds, about 15 pounds, about 16 pounds, about 17 pounds, about 18 pounds, about 19 pounds, or about 20 pounds).

The portable module 6 may include a handle, or other method of carrying the portable module 6. In some embodiments, the portable module 6 includes a strap, such that a subject may carry the portable module 6 over their shoulder or around the waist like a belt. In some embodiments, the portable module 6 further includes a fastener, such that the portable module 6 may be releasably secured to the body of a subject, e.g., a clip attached to a belt, or a garment.

### Pressure Source

The modular breast pump system 1 and/or the milk distribution system 28 can include a pressure source 9 (e.g., a negative pressure source and/or a positive pressure source). Specifically, the pressure source is configured to be used to move fluids, such as air, water, milk, and/or cleaning fluid, in the modular breast pump system 1 and/or the milk distribution system 28. In some embodiments, the modular breast pump system 1 and/or the milk distribution system 28 includes a plurality of pressure sources (e.g., 1, 2, 3, 4, 5, 6, 7, 8, 9 or 10). In some embodiments, at least one pressure source is a peristaltic pump. In some embodiments, at least one pressure source is a vacuum pump, e.g., which is capable of producing negative and positive pressure.

In some embodiments, the modular breast pump system 1 includes a negative pressure source that provides negative pressure to the breast cup 2, and thus the nipple of the breast in order to express milk. Additionally, the negative pressure source may be configured transport milk in the modular breast pump system 1, e.g., to expand and/or compress the pressure chamber 20 and/or the milk chamber 21 in specific breast cups. In some embodiments, the negative pressure source may be configured to provide pressure to any breast cup, not limited to the exemplary breast cup described herein. In some embodiments, the negative pressure source is a vacuum pump.

In some embodiments, the modular breast pump system 1 includes a positive pressure source that provides positive pressure to the breast cup 2 to aid in transportation of milk in the modular breast pump system, e.g., to expand and/or compress the pressure chamber 20 and/or the milk chamber 21 if present. In some embodiments, the positive pressure source is a pressure pump, a manually compressible chamber, a vacuum pump, a peristaltic pump, or a valve to the ambient atmosphere.

In some embodiments, pressure applied to the breast cup 2 varies. In some embodiments, the pressure source applies pressure to the breast cup 2 for 1 to 20 cycles (e.g., 1 to 2 cycles, 1 to 3 cycles, 1 to 4 cycles, 1 to 5 cycles, 1 to 6 cycles, 1 to 7 cycles, 1 to 8 cycles, 1 to 9 cycles, 1 to 10 cycles, 1 to 15 cycles, 2 to 3 cycles, 2 to 4 cycles, 2 to 5 cycles, 3 to 7 cycles, 4 to 6 cycles, 5 to 10 cycles, 5 to 15 cycles, 10 to 15 cycles, 15 to 20 cycles, 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, or 20 cycles). A first cycle may apply a maximum negative pressure of 0 mmHg to about 400 mmHg and/or a maximum positive pressure of about 0 mmHg to about 400 mmHg of positive pressure. A second cycle may apply a maximum negative pressure of about 0 mmHg to about 400 mmHg and/or a maximum positive pressure of about 0 mmHg to about 400 mmHg of positive pressure. Likewise, a third, fourth, fifth, etc., cycle, may apply a maximum negative pressure of about 0 mmHg to about 400 mmHg and/or a maximum positive pressure of about 0 mmHg to about 400 mmHg of positive pressure.

In some embodiments, the milk distribution system 28 includes a pressure source in fluidic communication with the breast cup 2, the manifold 29, and/or at least one of a plurality of reservoirs 3. In some embodiments, the milk distribution system 28 includes a pressure source configured to transport milk from the breast cup 2 to a reservoir 3. In some embodiments, this pressure source is the same pressure source used to pressurize the pressure chamber 20. In some embodiments, this pressure source is a different pressure source than is used to pressurize the pressure chamber 20. In some embodiments, this pressure source is a peristaltic pump. In some embodiments, milk distribution system 28 includes a fluid conduit 5 in fluidic communication with the breast cup 2, the manifold 29, at least one reservoir 3, and/or another fluid conduit 5 providing fluidic communication between components of the milk distribution system 28. In some embodiments, the pressure source 9 is a negative pressure source and/or a positive pressure source. In some embodiments, the pressure source is configured to open and/close an outlet of the manifold 31. In some embodiments, the pressure source is configured to open and/close an inlet of a reservoir, e.g., as shown in FIG. 12. In some embodiments, the inlet of a reservoir 3 is nominally closed and is configured to open upon pressurization provided by a pressure source.

The negative pressure source may provide a negative pressure from about 0 mmHg to about 400 mmHg (e.g., about 0 mmHg to about 10 mmHg, about 0 mmHg to about 20 mmHg, about 0 mmHg to about 30 mmHg, about 0 mmHg to about 40 mmHg, about 0 mmHg to about 50 mmHg, about 0 mmHg to about 60 mmHg, about 0 mmHg to about 70 mmHg, about 0 mmHg to about 80 mmHg, about 0 mmHg to about 90 mmHg, about 0 to about 100 mmHg, about 0 mmHg to about 110 mmHg, about 0 mmHg to about 120 mmHg, about 0 mmHg to about 130 mmHg, about 0 to about 140 mmHg, about 0 mmHg to about 150 mmHg, about 0 to about 175 mmHg, about 0 mmHg to about 200 mmHg, about 0 mmHg to about 225 mmHg, about 0 mmHg to about 250 mmHg, about 0 mmHg to about 275 mmHg, about 0 mmHg to about 300 mmHg, about 0 to about 350 mmHg, about 0 mmHg to about 400 mmHg, about 10 mmHg to about 20 mmHg, about 10 mmHg to about 30 mmHg, about 10 mmHg to about 40 mmHg, about 10 mmHg to about 50 mmHg, about 10 mmHg to about 60 mmHg, about 10 mmHg to about 70 mmHg, about 10 mmHg to about 80 mmHg, about 10 mmHg to about 90 mmHg, about 10 mmHg to about 100 mmHg, about 10 mmHg to about 110 mmHg, about 10 mmHg to about 120 mmHg, about 10 mmHg to about 130 mmHg, about 10 mmHg to about 140 mmHg, about 10 mmHg to about 150 mmHg, about 10 mmHg to about 175 mmHg, about 10 mmHg to about 200 mmHg, about 10 mmHg to about 225 mmHg, about 10 mmHg to about 250 mmHg, about 10 mmHg to about 300 mmHg, about 10 to about 350 mmHg, about 10 mmHg to about 400 mmHg, about 20 mmHg to about 30 mmHg, about 20 mmHg to about 40 mmHg, about 20 mmHg to about 50 mmHg, about 20 mmHg to about 60 mmHg, about 20 mmHg to about 70 mmHg, about 20 mmHg to about 80 mmHg, about 20 mmHg to about 90 mmHg, about 20 mmHg to about 100 mmHg, about 20 mmHg to about 110 mmHg, about 20 mmHg to about 120 mmHg, about 20 mmHg to about 130 mmHg, about 20 mmHg to about 140 mmHg, about 20 mmHg to about 150 mmHg, about 20 mmHg to about 175 mmHg, about 20 mmHg to about 200 mmHg, about 20 mmHg to about 225 mmHg, about 20 mmHg to about 250 mmHg, about 20 mmHg to about 300 mmHg, about 20 to about 350 mmHg, about 20 mmHg to about 400 mmHg, about 25 mmHg to about 50 mmHg, about 25 mmHg to about 75 mmHg, about 25 mmHg to about 100 mmHg, about 30 mmHg to about 40 mmHg, about 30 mmHg to about 50 mmHg, about 30 mmHg to about 60 mmHg, about 30 mmHg to about 70 mmHg, about 30 mmHg to about 80 mmHg, about 30 mmHg to about 90 mmHg, about 30 mmHg to about 100 mmHg, about 30 mmHg to about 110 mmHg, about 30 mmHg to about 120 mmHg, about 30 mmHg to about 130 mmHg, about 30 mmHg to about 140 mmHg, about 30 mmHg to about 150 mmHg, about 30 mmHg to about 175 mmHg, about 30 mmHg to about 200 mmHg, about 30 mmHg to about 225 mmHg, about 30 mmHg to about 250 mmHg, about 30 mmHg to about 300 mmHg, about 30 to about 350 mmHg, about 30 mmHg to about 400 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 40 mmHg to about 110 mmHg, about 40 mmHg to about 120 mmHg, about 40 mmHg to about 130 mmHg, about 40 mmHg to about 140 mmHg, about 40 mmHg to about 150 mmHg, about 40 mmHg to about 175 mmHg, about 40 mmHg to about 200 mmHg, about 40 mmHg to about 225 mmHg, about 40 mmHg to about 250 mmHg, about 40 mmHg to about 300 mmHg, about 40 to about 350 mmHg, about 40 mmHg to about 400 mmHg, about 50 mmHg to about 60 mmHg, about 50 mmHg to about 70 mmHg, about 50 mmHg to about 75 mmHg, about 50 mmHg to about 80 mmHg, about 50 mmHg to about 90 mmHg, about 50 mmHg to about 100 mmHg, about 50 mmHg to about 110 mmHg, about 50 mmHg to about 120 mmHg, about 50 mmHg to about 130 mmHg, about mmHg 50 to about 140 mmHg, about 50 mmHg to about 150 mmHg, about 50 mmHg to about 175 mmHg, about 50 mmHg to about 200 mmHg, about 50 mmHg to about 225 mmHg, about 50 mmHg to about 250 mmHg, about 50 mmHg to about 300 mmHg, about 50 to about 350 mmHg, about 50 mmHg to about 400 mmHg, about 60 mmHg to about 70 mmHg, about 60 mmHg to about 80 mmHg, about 60 mmHg to about 90 mmHg, about 60 mmHg to about 100 mmHg, about 60 mmHg to about 110 mmHg, about 60 mmHg to about 120 mmHg, about 60 mmHg to about 130 mmHg, about 60 mmHg to about 140 mmHg, about 60 mmHg to about 150 mmHg, about 60 mmHg to about 175 mmHg, about 60 mmHg to about 200 mmHg, about 60 mmHg to about 225 mmHg, about 60 mmHg to about 250 mmHg, about 60 mmHg to about 300 mmHg, about 60 to about 350 mmHg, about 60 mmHg to about 400 mmHg, about 70 mmHg to about 80 mmHg, about 70 mmHg to about 90 mmHg, about 70 mmHg to about 100 mmHg, about 70 mmHg to about 110 mmHg, about 70 mmHg to about 120 mmHg, about 70 mmHg to about 130 mmHg, about 70 mmHg to about 140 mmHg, about 70 mmHg to about 150 mmHg, about 70 mmHg to about 175 mmHg, about 70 mmHg to about 200 mmHg, about 70 mmHg to about 225 mmHg, about 70 mmHg to about 250 mmHg, about 70 mmHg to about 300 mmHg, about 70 to about 350 mmHg, about 70 mmHg to about 400 mmHg, about 75 mmHg to about 100 mmHg, about 75 mmHg to about 125 mmHg, about 80 mmHg to about 90 mmHg, about 80 mmHg to about 100 mmHg, about 80 mmHg to about 110 mmHg, about 80 mmHg to about 120 mmHg, about 80 mmHg to about 130 mmHg, about 80 mmHg to about 140 mmHg, about 80 mmHg to about 150 mmHg, about 80 mmHg to about 175 mmHg, about 80 mmHg to about 200 mmHg, about 80 mmHg to about 225 mmHg, about 80 mmHg to about 250 mmHg, about 80 mmHg to about 300 mmHg, about 80 to about 350 mmHg, about 80 mmHg to about 400 mmHg, about 90 mmHg to about 100 mmHg, about 90 mmHg to about 110 mmHg, about 90 mmHg to about 120 mmHg, about 90 mmHg to about 130 mmHg, about 90 mmHg to about 140 mmHg, about 90 mmHg to about 150 mmHg, about 90 mmHg to about 175 mmHg, about 90 mmHg to about 200 mmHg, about 90 mmHg to about 225 mmHg, about 90 mmHg to about 250 mmHg, about 90 mmHg to about 300 mmHg, about 90 to about 350 mmHg, about 90 mmHg to about 400 mmHg, about 100 mmHg to about 110 mmHg, about 100 mmHg to about 120 mmHg, about 100 mmHg to about 130 mmHg, about 100 mmHg to about 140 mmHg, about 100 mmHg to about 150 mmHg, about 100 mmHg to about 175 mmHg, about 100 mmHg to about 200 mmHg, about 100 mmHg to about 225 mmHg, about 100 mmHg to about 250 mmHg, about 100 mmHg to about 300 mmHg, about 100 to about 350 mmHg, about 100 mmHg to about 400 mmHg, about 110 mmHg to about 120 mmHg, about 110 mmHg to about 130 mmHg, about 110 mmHg to about 140 mmHg, about 110 mmHg to about 150 mmHg, about 110 mmHg to about 175 mmHg, about 110 mmHg to about 200 mmHg, about 110 mmHg to about 225 mmHg, about 110 mmHg to about 250 mmHg, about 110 mmHg to about 300 mmHg, about 110 to about 350 mmHg, about 110 mmHg to about 400 mmHg, about 120 mmHg to about 130 mmHg, about 120 mmHg to about 140 mmHg, about 120 mmHg to about 150 mmHg, about 120 mmHg to about 175 mmHg, about 120 mmHg to about 200 mmHg, about 120 mmHg to about 225 mmHg, about 120 mmHg to about 250 mmHg, about 120 mmHg to about 300 mmHg, about 120 to about 350 mmHg, about 120 mmHg to about 400 mmHg, about 130 mmHg to about 140 mmHg, about 130 mmHg to about 150 mmHg, about 130 mmHg to about 175 mmHg, about 130 mmHg to about 200 mmHg, about 130 mmHg to about 225 mmHg, about 130 mmHg to about 250 mmHg, about 130 mmHg to about 300 mmHg, about 130 to about 350 mmHg, about 130 mmHg to about 400 mmHg, about 140 mmHg to about 150 mmHg, about 140 mmHg to about 175 mmHg, about 140 mmHg to about 200 mmHg, about 140 mmHg to about 225 mmHg, about 140 mmHg to about 250 mmHg, about 140 mmHg to about 300 mmHg, about 140 to about 350 mmHg, about 140 mmHg to about 400 mmHg, about 150 mmHg to about 175 mmHg, about 150 mmHg to about 200 mmHg, about 150 mmHg to about 225 mmHg, about 150 mmHg to about 250 mmHg, about 150 mmHg to about 300 mmHg, about 150 to about 350 mmHg, about 150 mmHg to about 400 mmHg, about 175 mmHg to about 200 mmHg, about 175 mmHg to about 225 mmHg, about 175 mmHg to about 250 mmHg, about 175 mmHg to about 300 mmHg, about 175 to about 350 mmHg, about 175 mmHg to about 400 mmHg, about 200 mmHg to about 225 mmHg, about 200 mmHg to about 250 mmHg, about 200 mmHg to about 300 mmHg, about 200 to about 350 mmHg, about 200 mmHg to about 400 mmHg, about 225 mmHg to about 250 mmHg, about 225 mmHg to about 300 mmHg, about 225 to about 350 mmHg, about 225 mmHg to about 400 mmHg, about 275 mmHg to about 300 mmHg, about 275 to about 350 mmHg, about 275 mmHg to about 400 mmHg, about 300 mmHg to about 325 mmHg, about 300 to about 350 mmHg, about 300 mmHg to about 400 mmHg, about 325 mmHg to about 350 mmHg, about 325 to about 375 mmHg, about 325 mmHg to about 400 mmHg, about 350 mmHg to about 375 mmHg, about 350 to about 400 mmHg, or about 375 mmHg to about 400 mmHg.

The positive pressure source can provide a positive pressure from about 0 mmHg to about 400 mmHg (e.g., about 0 mmHg to about 10 mmHg, about 0 mmHg to about 20 mmHg, about 0 mmHg to about 30 mmHg, about 0 mmHg to about 40 mmHg, about 0 mmHg to about 50 mmHg, about 0 mmHg to about 60 mmHg, about 0 mmHg to about 70 mmHg, about 0 mmHg to about 80 mmHg, about 0 mmHg to about 90 mmHg, about 0 to about 100 mmHg, about 0 mmHg to about 110 mmHg, about 0 mmHg to about 120 mmHg, about 0 mmHg to about 130 mmHg, about 0 to about 140 mmHg, about 0 mmHg to about 150 mmHg, about 0 to about 175 mmHg, about 0 mmHg to about 200 mmHg, about 0 mmHg to about 225 mmHg, about 0 mmHg to about 250 mmHg, about 0 mmHg to about 275 mmHg, about 0 mmHg to about 300 mmHg, about 0 to about 350 mmHg, about 0 mmHg to about 400 mmHg, about 10 mmHg to about 20 mmHg, about 10 mmHg to about 30 mmHg, about 10 mmHg to about 40 mmHg, about 10 mmHg to about 50 mmHg, about 10 mmHg to about 60 mmHg, about 10 mmHg to about 70 mmHg, about 10 mmHg to about 80 mmHg, about 10 mmHg to about 90 mmHg, about 10 mmHg to about 100 mmHg, about 10 mmHg to about 110 mmHg, about 10 mmHg to about 120 mmHg, about 10 mmHg to about 130 mmHg, about 10 mmHg to about 140 mmHg, about 10 mmHg to about 150 mmHg, about 10 mmHg to about 175 mmHg, about 10 mmHg to about 200 mmHg, about 10 mmHg to about 225 mmHg, about 10 mmHg to about 250 mmHg, about 10 mmHg to about 300 mmHg, about 10 to about 350 mmHg, about 10 mmHg to about 400 mmHg, about 20 mmHg to about 30 mmHg, about 20 mmHg to about 40 mmHg, about 20 mmHg to about 50 mmHg, about 20 mmHg to about 60 mmHg, about 20 mmHg to about 70 mmHg, about 20 mmHg to about 80 mmHg, about 20 mmHg to about 90 mmHg, about 20 mmHg to about 100 mmHg, about 20 mmHg to about 110 mmHg, about 20 mmHg to about 120 mmHg, about 20 mmHg to about 130 mmHg, about 20 mmHg to about 140 mmHg, about 20 mmHg to about 150 mmHg, about 20 mmHg to about 175 mmHg, about 20 mmHg to about 200 mmHg, about 20 mmHg to about 225 mmHg, about 20 mmHg to about 250 mmHg, about 20 mmHg to about 300 mmHg, about 20 to about 350 mmHg, about 20 mmHg to about 400 mmHg, about 25 mmHg to about 50 mmHg, about 25 mmHg to about 75 mmHg, about 25 mmHg to about 100 mmHg, about 30 mmHg to about 40 mmHg, about 30 mmHg to about 50 mmHg, about 30 mmHg to about 60 mmHg, about 30 mmHg to about 70 mmHg, about 30 mmHg to about 80 mmHg, about 30 mmHg to about 90 mmHg, about 30 mmHg to about 100 mmHg, about 30 mmHg to about 110 mmHg, about 30 mmHg to about 120 mmHg, about 30 mmHg to about 130 mmHg, about 30 mmHg to about 140 mmHg, about 30 mmHg to about 150 mmHg, about 30 mmHg to about 175 mmHg, about 30 mmHg to about 200 mmHg, about 30 mmHg to about 225 mmHg, about 30 mmHg to about 250 mmHg, about 30 mmHg to about 300 mmHg, about 30 to about 350 mmHg, about 30 mmHg to about 400 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 40 mmHg to about 110 mmHg, about 40 mmHg to about 120 mmHg, about 40 mmHg to about 130 mmHg, about 40 mmHg to about 140 mmHg, about 40 mmHg to about 150 mmHg, about 40 mmHg to about 175 mmHg, about 40 mmHg to about 200 mmHg, about 40 mmHg to about 225 mmHg, about 40 mmHg to about 250 mmHg, about 40 mmHg to about 300 mmHg, about 40 to about 350 mmHg, about 40 mmHg to about 400 mmHg, about 50 mmHg to about 60 mmHg, about 50 mmHg to about 70 mmHg, about 50 mmHg to about 75 mmHg, about 50 mmHg to about 80 mmHg, about 50 mmHg to about 90 mmHg, about 50 mmHg to about 100 mmHg, about 50 mmHg to about 110 mmHg, about 50 mmHg to about 120 mmHg, about 50 mmHg to about 130 mmHg, about mmHg 50 to about 140 mmHg, about 50 mmHg to about 150 mmHg, about 50 mmHg to about 175 mmHg, about 50 mmHg to about 200 mmHg, about 50 mmHg to about 225 mmHg, about 50 mmHg to about 250 mmHg, about 50 mmHg to about 300 mmHg, about 50 to about 350 mmHg, about 50 mmHg to about 400 mmHg, about 60 mmHg to about 70 mmHg, about 60 mmHg to about 80 mmHg, about 60 mmHg to about 90 mmHg, about 60 mmHg to about 100 mmHg, about 60 mmHg to about 110 mmHg, about 60 mmHg to about 120 mmHg, about 60 mmHg to about 130 mmHg, about 60 mmHg to about 140 mmHg, about 60 mmHg to about 150 mmHg, about 60 mmHg to about 175 mmHg, about 60 mmHg to about 200 mmHg, about 60 mmHg to about 225 mmHg, about 60 mmHg to about 250 mmHg, about 60 mmHg to about 300 mmHg, about 60 to about 350 mmHg, about 60 mmHg to about 400 mmHg, about 70 mmHg to about 80 mmHg, about 70 mmHg to about 90 mmHg, about 70 mmHg to about 100 mmHg, about 70 mmHg to about 110 mmHg, about 70 mmHg to about 120 mmHg, about 70 mmHg to about 130 mmHg, about 70 mmHg to about 140 mmHg, about 70 mmHg to about 150 mmHg, about 70 mmHg to about 175 mmHg, about 70 mmHg to about 200 mmHg, about 70 mmHg to about 225 mmHg, about 70 mmHg to about 250 mmHg, about 70 mmHg to about 300 mmHg, about 70 to about 350 mmHg, about 70 mmHg to about 400 mmHg, about 75 mmHg to about 100 mmHg, about 75 mmHg to about 125 mmHg, about 80 mmHg to about 90 mmHg, about 80 mmHg to about 100 mmHg, about 80 mmHg to about 110 mmHg, about 80 mmHg to about 120 mmHg, about 80 mmHg to about 130 mmHg, about 80 mmHg to about 140 mmHg, about 80 mmHg to about 150 mmHg, about 80 mmHg to about 175 mmHg, about 80 mmHg to about 200 mmHg, about 80 mmHg to about 225 mmHg, about 80 mmHg to about 250 mmHg, about 80 mmHg to about 300 mmHg, about 80 to about 350 mmHg, about 80 mmHg to about 400 mmHg, about 90 mmHg to about 100 mmHg, about 90 mmHg to about 110 mmHg, about 90 mmHg to about 120 mmHg, about 90 mmHg to about 130 mmHg, about 90 mmHg to about 140 mmHg, about 90 mmHg to about 150 mmHg, about 90 mmHg to about 175 mmHg, about 90 mmHg to about 200 mmHg, about 90 mmHg to about 225 mmHg, about 90 mmHg to about 250 mmHg, about 90 mmHg to about 300 mmHg, about 90 to about 350 mmHg, about 90 mmHg to about 400 mmHg, about 100 mmHg to about 110 mmHg, about 100 mmHg to about 120 mmHg, about 100 mmHg to about 130 mmHg, about 100 mmHg to about 140 mmHg, about 100 mmHg to about 150 mmHg, about 100 mmHg to about 175 mmHg, about 100 mmHg to about 200 mmHg, about 100 mmHg to about 225 mmHg, about 100 mmHg to about 250 mmHg, about 100 mmHg to about 300 mmHg, about 100 to about 350 mmHg, about 100 mmHg to about 400 mmHg, about 110 mmHg to about 120 mmHg, about 110 mmHg to about 130 mmHg, about 110 mmHg to about 140 mmHg, about 110 mmHg to about 150 mmHg, about 110 mmHg to about 175 mmHg, about 110 mmHg to about 200 mmHg, about 110 mmHg to about 225 mmHg, about 110 mmHg to about 250 mmHg, about 110 mmHg to about 300 mmHg, about 110 to about 350 mmHg, about 110 mmHg to about 400 mmHg, about 120 mmHg to about 130 mmHg, about 120 mmHg to about 140 mmHg, about 120 mmHg to about 150 mmHg, about 120 mmHg to about 175 mmHg, about 120 mmHg to about 200 mmHg, about 120 mmHg to about 225 mmHg, about 120 mmHg to about 250 mmHg, about 120 mmHg to about 300 mmHg, about 120 to about 350 mmHg, about 120 mmHg to about 400 mmHg, about 130 mmHg to about 140 mmHg, about 130 mmHg to about 150 mmHg, about 130 mmHg to about 175 mmHg, about 130 mmHg to about 200 mmHg, about 130 mmHg to about 225 mmHg, about 130 mmHg to about 250 mmHg, about 130 mmHg to about 300 mmHg, about 130 to about 350 mmHg, about 130 mmHg to about 400 mmHg, about 140 mmHg to about 150 mmHg, about 140 mmHg to about 175 mmHg, about 140 mmHg to about 200 mmHg, about 140 mmHg to about 225 mmHg, about 140 mmHg to about 250 mmHg, about 140 mmHg to about 300 mmHg, about 140 to about 350 mmHg, about 140 mmHg to about 400 mmHg, about 150 mmHg to about 175 mmHg, about 150 mmHg to about 200 mmHg, about 150 mmHg to about 225 mmHg, about 150 mmHg to about 250 mmHg, about 150 mmHg to about 300 mmHg, about 150 to about 350 mmHg, about 150 mmHg to about 400 mmHg, about 175 mmHg to about 200 mmHg, about 175 mmHg to about 225 mmHg, about 175 mmHg to about 250 mmHg, about 175 mmHg to about 300 mmHg, about 175 to about 350 mmHg, about 175 mmHg to about 400 mmHg, about 200 mmHg to about 225 mmHg, about 200 mmHg to about 250 mmHg, about 200 mmHg to about 300 mmHg, about 200 to about 350 mmHg, about 200 mmHg to about 400 mmHg, about 225 mmHg to about 250 mmHg, about 225 mmHg to about 300 mmHg, about 225 to about 350 mmHg, about 225 mmHg to about 400 mmHg, about 275 mmHg to about 300 mmHg, about 275 to about 350 mmHg, about 275 mmHg to about 400 mmHg, about 300 mmHg to about 325 mmHg, about 300 to about 350 mmHg, about 300 mmHg to about 400 mmHg, about 325 mmHg to about 350 mmHg, about 325 to about 375 mmHg, about 325 mmHg to about 400 mmHg, about 350 mmHg to about 375 mmHg, about 350 to about 400 mmHg, or about 375 mmHg to about 400 mmHg.

In some embodiments, pressure applied to the breast cup 2 may change, e.g., from about every 0.1 seconds to about every 120 seconds (e.g., about every 0.1 seconds to about every 1 second, about every 0.1 seconds to about every 2 seconds, about every 0.1 seconds to about every 3 seconds, about every 0.1 seconds to about every 4 seconds, about every 0.1 seconds to about every 5 seconds, about every 0.1 seconds to about every 6 seconds, about every 0.1 seconds to about every 7 seconds, about every 0.1 seconds to about every 8 seconds, about every 0.1 seconds to about every 9 seconds, about every 0.1 seconds to about every 10 seconds, about every 0.1 seconds to about every 11 seconds, about every 0.1 seconds to about every 12 seconds, about every 0.1 seconds to about every 13 seconds, about every 0.1 seconds to about every 14 seconds, about every 0.1 seconds to about every 15 seconds, about every 0.1 seconds to about every 20 seconds, about every 0.1 seconds to about every 30 seconds, about every 0.1 seconds to about every 45 seconds, about every 0.1 seconds to about every 60 seconds, about every 0.1 seconds to about every 90 seconds, about every 1 second to about every 2 seconds, about every 1 second to about every 3 seconds, about every 1 second to about every 4 seconds, about every 1 second to about every 5 seconds, about every 1 second to about every 6 seconds, about every 1 second to about every 7 seconds, about every 1 second to about every 8 seconds, about every 1 second to about every 9 seconds, about every 1 second to about every 10 seconds, about every 2 second to about every 5 seconds, about every 3 second to about every 7 seconds, about every 4 second to about every 5 seconds, about every 5 second to about every 10 seconds, about every 10 second to about every 15 seconds, about every 15 second to about every 30 seconds, about every 0.1 seconds, about every 0.2 seconds, about every 0.3 seconds, about every 0.4 seconds, about every 0.5 seconds, about every 0.6 seconds, about every 0.7 seconds, about every 0.8 seconds, about every 0.9 seconds, about every 1 second, about every 1.5 seconds, about every 2 seconds, about every 3 seconds, about every 4 seconds, about every 5 seconds, about every 6 seconds, about every 7 seconds, about every 8 seconds, about every 9 seconds, about every 10 seconds, about every 11 seconds, about every 12 seconds, about every 13 seconds, about every 14 seconds, about every 15 seconds, about every 20 seconds, about every 30 seconds, about every 45 seconds, about every 60 seconds, about every 75 seconds, about every 90 seconds, about every 105 seconds, or about every 120 seconds).

In some embodiments, pressure applied to the breast cup 2 changes gradually. In some embodiments, pressure applied to the breast cup 2 changes at different rates, e.g., in a first cycle the pressure applied to the breast cup 2 changes over about 0.1 seconds to about 120 seconds (e.g., about 1 second), and in a second cycle the pressure applied to the breast cup 2 changes over about 0.1 seconds to about 120 seconds (e.g., about 5 seconds).

Pressure within the breast cup 2 can provide a pulsating or massaging sensation. In some embodiments, the speed and pressure of the pulsation or massage may be selected by the user. In some embodiments, alternating pressure mimics the action of suckling.

### Reservoir

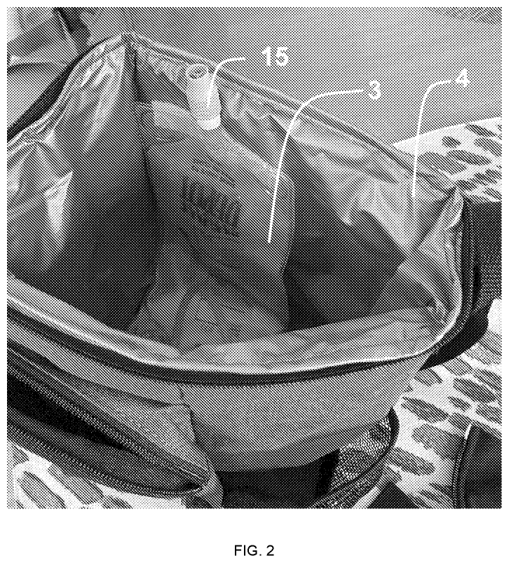

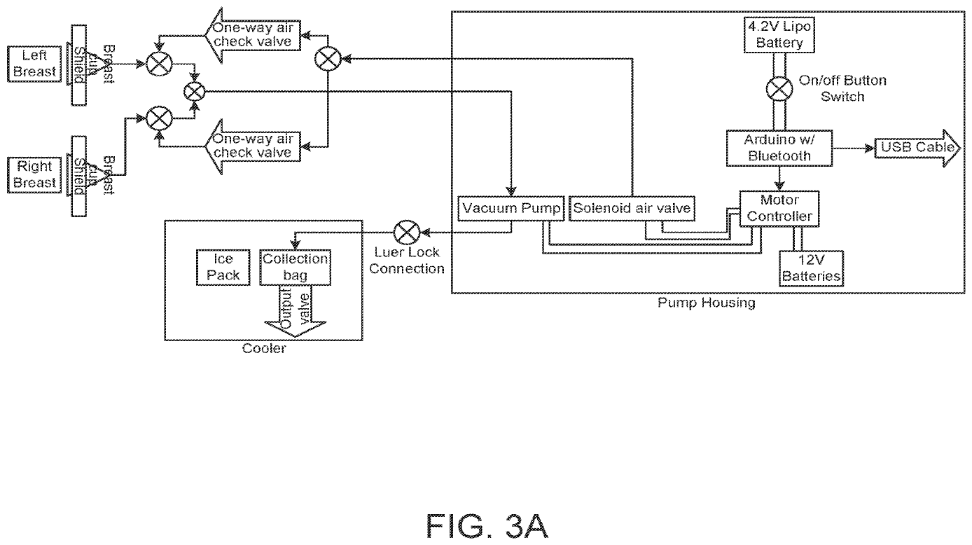

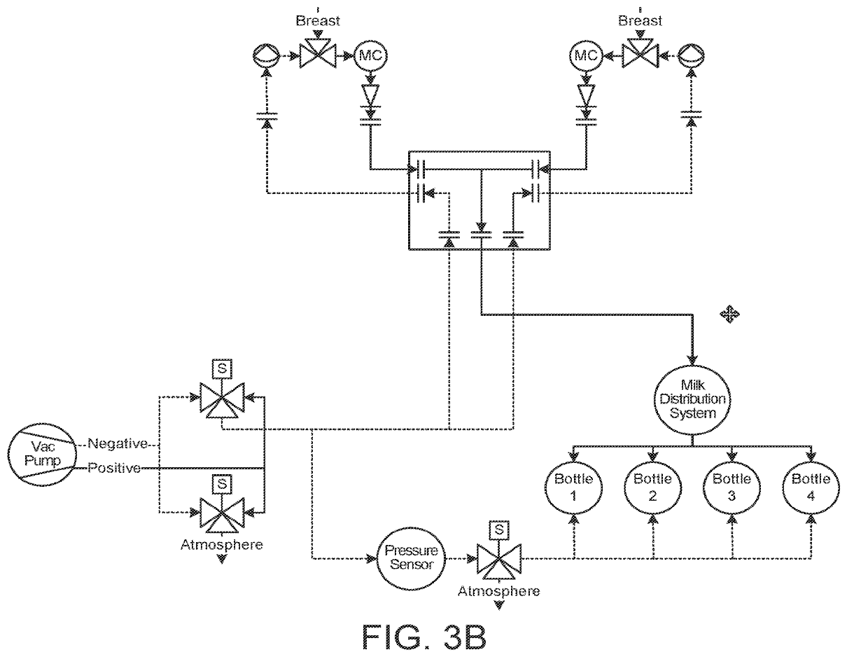

The modular breast pump system 1 and/or the milk distribution system 28 may include a reservoir 3, e.g., as shown in FIG. 2. The reservoir 3 is a collection container for the milk, such as a bottle or bag. The reservoir 3 may be polymeric or fabric. The reservoir 3 may be in fluidic communication with the pressure source 9, such that fluid is moved from the breast, through the pressure source 9, to the reservoir 3. The reservoir 3 may be releasably connected to a fluid conduit 5, such as a tube. The reservoir 3 may be configured for ease of cleaning. The reservoir 3 may be placed in a dishwasher, refrigerator, and/or freezer. In some embodiments, the reservoir 3 includes a temperature control element 47 (e.g., a cooling element and/or insulation). In some embodiments, the reservoir 3 includes a refrigerant, e.g., a thermoelectric cooler or an ice pack. The reservoir enclosure may also include a thermostat to regulate the temperature. In some embodiments, the reservoir 3 is insulated, e.g., with foam or a reflective material. Typically, reservoirs 3 are removable from the system, e.g., to feed an infant.

In some embodiments, the reservoir includes a volume from about 50 mL to about 1500 mL (e.g., about 50 mL to about 100 mL, about 50 mL to about 150 mL, about 50 mL to about 200 mL, about 50 mL to about 250 mL, about 50 mL to about 300 mL, about 50 mL to about 400 mL, about 50 mL to about 500 mL, about 50 mL to about 600 mL, about 50 mL to about 700 mL, about 50 mL to about 700 mL, about 50 mL to about 750 mL, about 50 mL to about 800 mL, about 50 mL to about 900 mL, about 50 mL to about 1000 mL, about 50 mL to about 1100 mL, about 50 mL to about 1200 mL, about 50 mL to about 1300 mL, about 50 mL to about 1400 mL, about 100 mL to about 250 mL, about 100 mL to about 500 mL, about 100 mL to about 750 mL, about 100 mL to about 1000 mL, about 100 mL to about 1250 mL, about 250 mL to about 500 mL, about 250 mL to about 750 mL, about 250 mL to about 1000 mL, about 250 mL to about 1250 mL, about 250 mL to about 1500 mL, about 500 mL to about 750 mL, about 500 mL to about 1000 mL, about 500 mL to about 1250 mL, about 500 mL to about 1500 mL, about 750 mL to about 1000 mL, about 750 mL to about 1250 mL, about 750 mL to about 1500 mL, about 1000 mL to about 1250 mL, about 1250 mL to about 1500 mL, about 50 mL, about 100 mL to about 150 mL, about 200 mL, about 250 mL, about 300 mL, about 350 mL, about 400 mL, about 450 mL, about 500 mL, about 55 mL, about 600 mL, about 650 mL, about 700 mL, about 750 mL, about 800 mL, about 850 mL, about 900 mL, about 950 mL, about 1000 mL, about 1050 mL, about 1100 mL, about 1150 mL, about 1200 mL, about 1250 mL, about 1300 mL, about 1350 mL, about 1400 mL, about 1450 mL, or about 1500 mL).

In some embodiments, the modular breast pump system 1 and/or the milk distribution system 28 includes a plurality of reservoirs 3. In some embodiments, modular breast pump system includes from 1 to 10 reservoirs 3 (e.g., from 1 to 2, from 1 to 3, from 1 to 4, from 1 to 5, from 1 to 6, from 1 to 7, from 1 to 8, from 1 to 9, from 2 to 3, from 2 to 4, from 2 to 5, from 2 to 6, from 2 to 7, from 2 to 8, from 3 to 4, from 3 to 5, from 3 to 6, from 4 to 5, from 4 to 6, from 4 to 8, from 4 to 10, from 5 to 8, from 5 to 10, 1, 2, 3, 4, 5, 6, 7, 8, 9, or 10). In some embodiments, one reservoir may be disposed in the portable module 6, and an additional plurality of reservoirs may be disposed in an auxiliary reservoir enclosure 4. In this embodiment, the auxiliary reservoir enclosure 4, e.g., a cooler, may be an unattached to the portable module 6. In some embodiments, one reservoir may be disposed in the portable module 6, and an additional plurality of reservoirs may be disposed in the base module 7. In some embodiments, a plurality of reservoirs may be disposed in the portable module 6, the base module 7, and/or an auxiliary reservoir enclosure. In some embodiments, the base module 7 further includes the breast milk distribution system 28, which further includes from 2 to 12 reservoirs 3. In some embodiments, the breast milk distribution system 28 further includes from 4 to 6 reservoirs 3. In some embodiments, the plurality of reservoirs 3 is configured to be in fluidic communication with the breast cup.

In some embodiments, a reservoir may be releasably attached to the portable module 6. In some embodiments, the reservoir is disposed in a reservoir enclosure 4 within the portable module 6, wherein the enclosure is physically and/or thermally separate from other components of the portable module 6, e.g., the pressure source. In some embodiments, the reservoir is configured to be connected to the base unit in the absence of a reservoir enclosure, e.g., held by a strap or pocket on the exterior of the portable module 6. In some embodiments, the reservoir may be releasably connected to the breast cup and/or one or more fluidic conduits. In some embodiments, the reservoir may be disposed separately from the portable module 6. For example, the reservoir may be connected to the portable module 6 via an opening in the reservoir where milk may enter the reservoir. In some embodiments, the reservoir may be fluidically connected to the portable module 6. In some embodiments, the reservoir may include a catch, or internal and/or external threads such that it may be releasably attached to the portable module 6. In some embodiments, the reservoir may be releasably attached to the portable module 6 via a catch mechanism, latch, snap fit, or other suitable mechanism.

In some embodiments, each reservoir 3 includes a lid, wherein lid includes the pressure outlet, the inlet of the reservoir 3, and/or the valve of the reservoir 3. The lid can be integral to or attached to the reservoir 3. In some embodiments, the lid is releasably attached to the reservoir 3. In some embodiments, the lid is screwed or snapped onto the reservoir 3. In particular, the reservoir 3 may include external threads, the lid may include internal threads, and the internal threads and the external threads may provide releasable attachment of the reservoir 3 and lid. In some embodiments, the lid is attached with a magnet, e.g., an electromagnet. In some embodiments, the lid is configured to be opened and/or closed with the electromagnet. In some embodiment, the lid is configured to be opened and/or closed with pressure, e.g., air pressure.

The lid of the reservoir can have a substantially flat or rounded top surface.

In some embodiments, the reservoir 3, e.g., the lid of the reservoir 3, includes a pressure outlet. The pressure outlet may or may not include a valve 24. In some embodiments, the pressure outlet is a breather tube. In some embodiments, the pressure outlet is an orifice, e.g., the reservoir 3 or the lid of the reservoir 3 may include an orifice. In some embodiments, the milk distribution system 28 includes a pressure outlet in fluidic communication with each reservoir 3. A pressure outlet is advantageous in allowing excess pressure to vent as the reservoir 3 is being filled with milk. In some embodiments, the pressure outlet is solely open as the reservoir 3 is being filled, and the pressure outlet is otherwise nominally closed. An outlet may be connected to a negative pressure source and a vent, e.g., to remove warm air to aid in cooling or to provide suction for milk flow.

### Reservoir Disposition

The modular breast pump system 1 may include a reservoir enclosure 4. The reservoir 3 may be within a reservoir enclosure 4, e.g., as shown in FIG. 2. In some embodiments, a plurality of reservoirs 3 is disposed in an enclosure 4. A reservoir 3 may be placed in the enclosure 4 prior to a breast pumping session. In some embodiments, the portable module 6 includes only a single reservoir.

In some embodiments, the modular breast pump system may include a plurality of reservoir enclosures 4. In some embodiments, the modular breast pump system 1 may include a first reservoir enclosure and a second reservoir enclosure. In some embodiments, a first reservoir enclosure 4 may be disposed within the portable module 6, e.g., such that a reservoir 3 may be releasably secured within the portable module 6. In some embodiments, a second reservoir enclosure 4 may be disposed within the base module 7. In some embodiments, the modular breast pump system 1 may include a third reservoir enclosure. In another embodiment, a third reservoir enclosure 4 may be an auxiliary reservoir enclosure, e.g., a cooler, wherein a plurality of reservoirs 3 may be disposed within the third reservoir enclosure 4. In some embodiments, the modular breast pump system may include a first reservoir enclosure disposed in the portable module 6, a second reservoir enclosure disposed in the base module 7, and/or a third auxiliary reservoir enclosure. In some embodiments, a reservoir enclosure in the portable module 6 and a reservoir enclosure in the base module 7 may form a single reservoir enclosure when the modules are mated. In some embodiments, a reservoir enclosure in the portable module 6 and a reservoir enclosure in the base module 7 are separate when the modules are mated.

The enclosure 4 is advantageous in that milk may be stored for a prolonged period of time, e.g., at least 10 minutes, at least 15 minutes, at least 20 minutes, at least 30 minutes, at least 40 minutes, at least 45 minutes, at least 60 minutes, at least 1.5 hours, at least 2 hours, at least 3 hours, at least 4 hours, at least 5 hours, at least 6 hours, at least 7 hours, at least 8 hours, at least 9 hours, or at least 10 hours.

The enclosure 4 may allow the milk to safely cool for prolonged storage. In some embodiments, the enclosure 4 may maintain the milk below about 6 °C (e.g., about 5 °C, about 5.5 °C, about 4.5 °C, about 4 °C, about 3.5 °C, about 3 °C, about 2.5 °C, about 2 °C, about 1.5 °C, about 1 °C, about 0.5 °C, or about 0 °C) for a prolonged period of time (e.g., at least 10 minutes, at least 15 minutes, at least 20 minutes, at least 30 minutes, at least 40 minutes, at least 45 minutes, at least 60 minutes, at least 1.5 hours, at least 2 hours, at least 3 hours, at least 4 hours, at least 5 hours, at least 6 hours, at least 7 hours, at least 8 hours, at least 9 hours, or at least 10 hours).

In some embodiments, the enclosure 4 includes a cooling element. The cooling element may be an ice pack and may also include insulation or heat resistant material. In some embodiments, the enclosure 4 includes a plurality of ice packs, such as from 2 to 20 (e.g., 2 to 3, 2 to 4, 2 to 5, 2 to 6, 2 to 7, 2 to 8, 2 to 9, 2 to 10, 2 to 11, 2 to 12, 3 to 4, 3 to 5, 3 to 6, 2 to 8, 3 to 10, 4 to 5, from 4 to 6, 4 to 8, 4 to 10, 4 to 12, 5 to 10, 6 to 8, 6 to 10, 6 to 12, 8 to 10, 8 to 12, 10 to 12, 10 to 20, 1, 2, 3, 4, 5, 6, 7, 8, 9, 10, 11, 12, 13, 14, 15, 16, 17, 18, 19, or 20). The ice pack may be stored in a freezer and then placed in the enclosure before the initiation of breast pumping.

In some embodiments, the cooling element substantially surrounds the reservoir 3. In some embodiments, the cooling element, e.g., the ice pack, is in contact with at least about 5% of the reservoir 3 (e.g., about 10%, about 20%, about 25%, about 30%, about 35%, about 40%, about 45%, about 50%, about 60%, about 70%, about 75%, about 80%, about 90%, or about 95%). In some embodiments, the cooling element conforms to the reservoir 3. In some embodiments, the cooling element is deformable ice pack.

In some embodiments, the enclosure 4 includes an internal recess, in which the reservoir 3 is configured to be placed in the recess. In some embodiments, following placement in the recess, the outer surface of the reservoir 3 is in contact with an internal surface of the recess. In some embodiments, at least about 5% (e.g., about 10%, about 20%, about 25%, about 30%, about 35%, about 40%, about 45%, about 50%, about 60%, about 70%, about 75%, about 80%, about 90%, or about 95%) of the outer surface of the reservoir 3 is in contact with the inner surface of the recess.

In some embodiments, the enclosure 4 includes a releasably attached tray, in which the tray includes a plurality of recesses (e.g., 2 to 3, 2 to 4, 2 to 5, 2 to 6, 2 to 7, 2 to 8, 2 to 9, 2 to 10, 2 to 11, 2 to 12, 3 to 4, 3 to 5, 3 to 6, 2 to 8, 3 to 10, 4 to 5, from 4 to 6, 4 to 8, 4 to 10, 4 to 12, 5 to 10, 6 to 8, 6 to 10, 6 to 12, 8 to 10, 8 to 12, 10 to 12, 10 to 20, 1, 2, 3, 4, 5, 6, 7, 8, 9, 10, 11, 12, 13, 14, 15, 16, 17, 18, 19, or 20). In some embodiments, the tray includes the cooling element. In some embodiments, the tray includes an ice pack. In some embodiments, the tray is substantially formed from an ice pack.

In some embodiments, the reservoir 3 includes a recess. In some embodiments, the cooling element, e.g., the ice pack, is configured to be placed in the recess. In some embodiments, at least about 5% (e.g., about 10%, about 20%, about 25%, about 30%, about 35%, about 40%, about 45%, about 50%, about 60%, about 70%, about 75%, about 80%, about 90%, or about 95%) of the outer surface of the cooling element is in contact with the inner surface of the recess.

In some embodiments, the enclosure 4 is a cooler, e.g., as shown in FIG. 2. In some embodiments, the enclosure 4 is electrically connected to a power source 12. In some embodiments, the power source is a rechargeable or single use battery. In some embodiments, the power source is an AC power source. In some embodiments, the enclosure 4 includes a refrigerant, e.g., a thermoelectric cooler. The reservoir enclosure may also include a thermostat to regulate the temperature.

In some embodiments, the enclosure includes a load cell or weight sensor, e.g., a scale. In some embodiments, the weight sensor may be disposed underneath the reservoir 3, such that the weight of the reservoir 3 is measured. In some embodiments, the weight of the reservoir 3 is measured over time using the weight sensor or load cell. In some embodiments, the weight of the reservoir 3 over time may be used to calculate flow rate of milk during a pumping session. In some embodiments, the weight of the reservoir 3 may be used to determine the volume of milk in the reservoir 3, the identity of reservoirs 3 filled or empty, or when a pumping cycle is complete.

The reservoir may also be attached to the exterior of the portable module 6, e.g., held in an exterior pocket or by a strap, or disposed separately from the accessory or portable unit.

The reservoir 3 or enclosure 4 may be wearable, such as including at least one fastener, e.g., a clip and/or strap, to secure the reservoir 3 or enclosure 4 to the user, e.g., the waist of a user. The present system is advantageous in that it may be used to breast pump while resting or sleeping and also used while going about daily activities, such as working, caring for an infant, etc. by mating or unmating (separating) the modules.

### Breast Cup

The modular breast pump system may be employed with any suitable breast cup that can be in fluidic communication with the system, e.g., via a fluidic conduit. As is known in the art, breast cups may include a breast shield component that is designed to contact the breast and surround the nipple. Breast cups may also include a housing to surround the breast shield. The breast cup may be actuated manually or by automation, e.g., by pressure cycles from a pump or other pressure source. The system is advantageous for automated pumping as it allows for the breast cup to stay in place. Pumping milk into a reservoir may occur by the action of a diaphragm, when present. Other pumping mechanisms may also be employed, including providing positive pressure breast cup, e.g., by forced air or providing negative pressure to a fluid conduit between the breast cup and a reservoir. Negative pressure may be employed by a peristaltic pump or by air being pulled into the fluid conduit. These may also act to flush the conduit after a pumping session or after a set number of pumping session. For example, a flush line may connect to the fluid conduit, with both the flush line and the fluid conduit having one directional valves to prevent back flow. Negative pressure may be applied by reducing pressure in a reservoir, and a vent may be present to release pressure when desired.

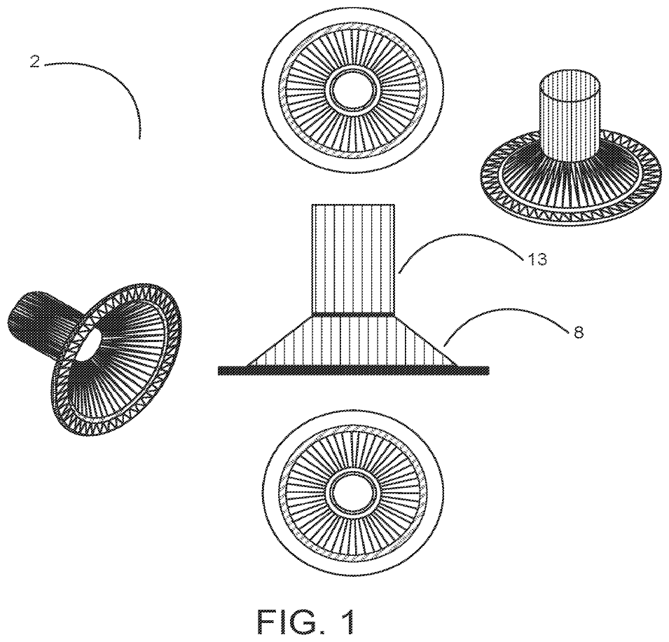

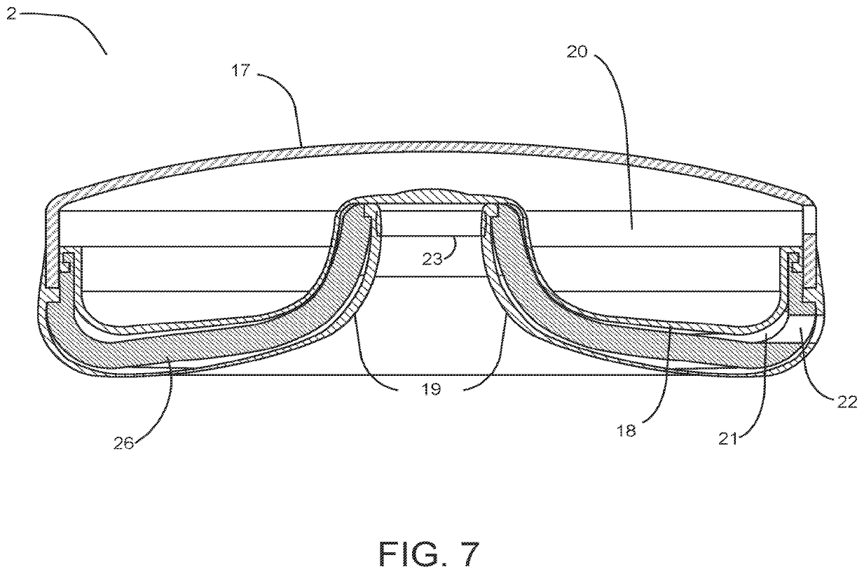

In some aspects, the modular breast pump system 1 may be used with any embodiment of the breast cups described in this disclosure. An exemplary breast cup 2 is shown in FIG. 1. The breast cup may include a housing (e.g., the upper housing 17 and the lower housing or milk chamber wall 26), a breast shield 19, and a diaphragm 18 disposed between the upper housing 17 and the breast shield 19 (as well as between the upper housing 17 and the lower housing or milk chamber wall 26), such that the diaphragm 18 separates a pressure chamber 20 from a milk chamber 21. In some embodiments, the breast cup 2 includes a milk chamber wall 26, e.g., as shown in FIG. 7. The milk chamber wall 26 can be disposed between the diaphragm 18 and the breast shield 19, thereby defining the milk chamber 21 between the diaphragm 18 and the milk chamber wall 26. Pressurization and/or depressurization of the pressure chamber 20 deforms the diaphragm 18, and thereby expands or compresses the milk chamber 21. The pressure chamber is not fluidically connected to the milk chamber or breast shield. In some embodiments, the pressure chamber is not in fluidic communication with the milk chamber or breast shield.

One or more of the components of the breast cup 2, including the upper housing 17, the diaphragm 18, the breast shield 19, and/or the milk chamber wall 26 may be removably attached to the breast cup 2. In some embodiments, the pressure chamber 20 and/or the milk chamber 21 are distinct reservoirs, as opposed to chambers formed from the space between other components (e.g., the upper housing 17, the diaphragm 18, the milk chamber wall 26, and/or the breast shield 19, and may be reversibly attached to the breast cup 2. For example, the milk chamber 21 may be removed from the breast cup 2 to be cleaned or replaced.

In some embodiments, at least one of the upper housing 17, the diaphragm 18, the breast shield 19, and/or the milk chamber wall 26 can include a protrusion and/or a recess, such as a groove. In some embodiments, a protrusion on at least one of the upper housing 17, the diaphragm 18, the breast shield 19, and/or the milk chamber wall 26 is configured to mate with a recess, such as a groove, on at least one of the upper housing 17, the diaphragm 18, the breast shield 19, and/or the milk chamber wall 26. Mating between a protrusion and a recess, e.g., groove, allows two components to be snapped, slid, or friction-fit together. FIG. 7 shows an embodiment of the breast cup 2, in which the upper housing 17, the diaphragm 18, the breast shield 19, and/or the milk chamber wall 26 are attached together at least partially using protrusions and recesses, e.g., grooves.

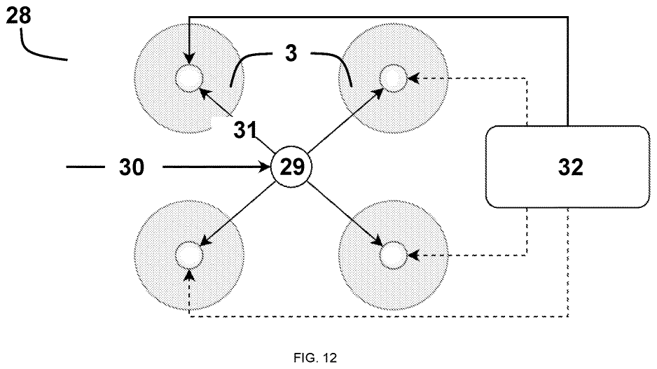

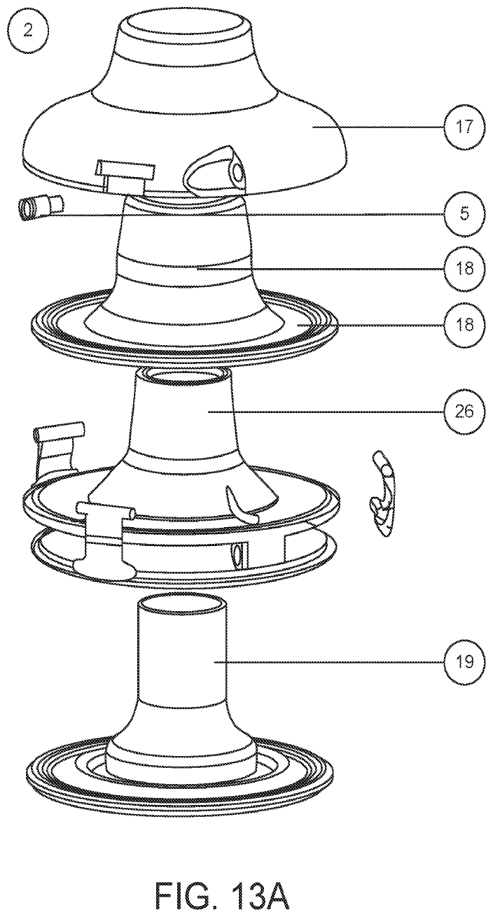

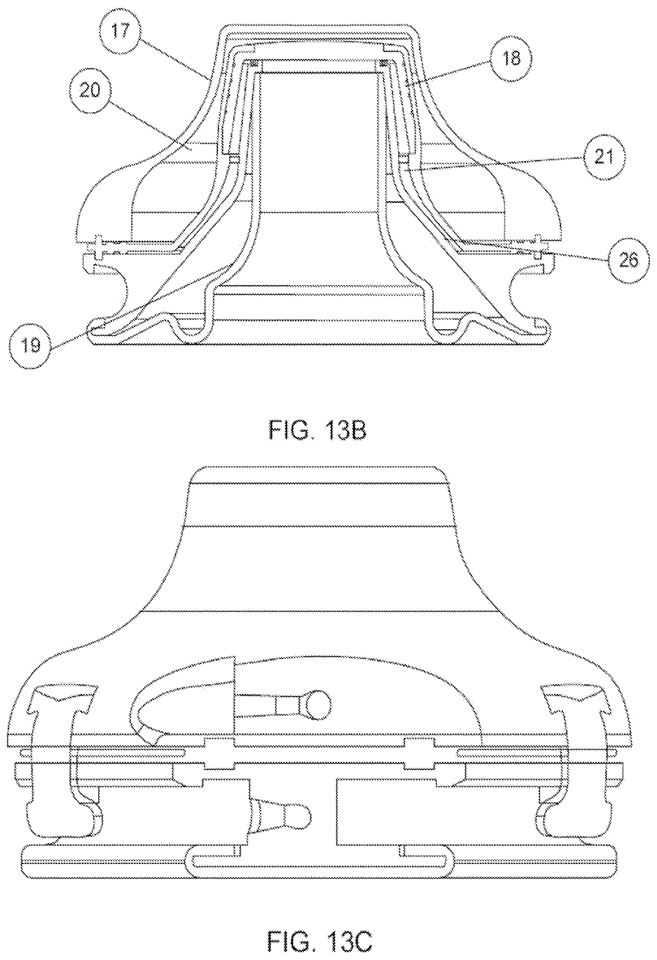

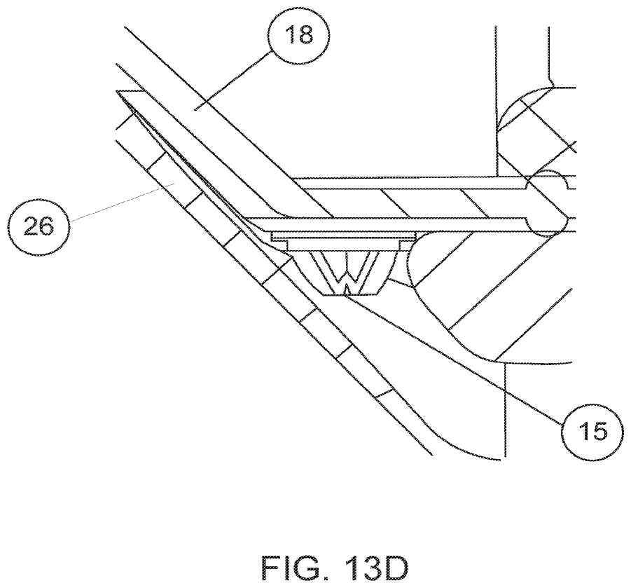

Alternatively, or in addition, at least one of the upper housing 17, the diaphragm 18, the breast shield 19, and/or the milk chamber wall 26 can be attached together using any other suitable connector, e.g., an adhesive (e.g., glue) or a fastener (e.g., a screw). FIGS. 13A-13D show an embodiment of the breast cup 2, in which the upper housing 17, the diaphragm 18, the breast shield 19, and/or the milk chamber wall 26 are attached together at least partially using a fastener.

The breast cup 2, e.g., via the breast shield 19, contacts the breast of the user and can provide a seal during breast pumping, such that milk does not leak. The breast cup 2, the breast shield 19, and/or the housing are configured to conform to the breast comfortably for frequent and/or long periods of wear, such that the breast is not irritated. For example, the interior of the breast cup 2, e.g., the breast shield 19, may conform to the breast and/or the nipple of the user.

The breast cup 2, the breast shield 19, and/or the housing may be in a shape for improved conformance to the breast, e.g., a funnel, a cone, or a dome. The exterior of the breast cup 2 may be rounded. A rounded breast cup 2 may appear breast-like, e.g., to be worn discreetly under clothing. The breast cup 2, the breast shield 19, and/or the housing may be toroidal, e.g., forming a ring, with the breast cup 2, the breast shield 19, and/or the housing covering the breast.

In some embodiments, the breast cup 2 is funneled. The breast cup 2 may include a wide portion 8 and a nipple tunnel 13, e.g., as shown in FIG. 1, in which the wide portion 8, e.g., of the breast cup 19, initially contacts the breast, and the nipple tunnel 13, e.g., of the breast cup 19, is configured to receive the nipple.

The wide portion 8 of the breast cup 2 may have a largest diameter from about 50 mm to about 250 mm (e.g., about 50 mm to about 75 mm, about 50 mm to about 100 mm, about 50 mm to about 125 mm, about 50 mm to about 150 mm, about 50 mm to about 75 mm, about 75 mm to about 100 mm, about 75 mm to about 125 mm, about 75 mm to about 150 mm, about 75 mm to about 175 mm, about 75 mm to about 200 mm, about 100 mm to about 125 mm, about 100 mm to about 150 mm, about 100 mm to about 175 mm, about 100 mm to about 200 mm, about 125 mm to about 150 mm, about 125 mm to about 175 mm, about 125 mm to about 200 mm, about 150 mm to about 175 mm, about 150 mm to about 200 mm, about 150 mm to about 250 mm, or about 200 mm to about 250 mm). The nipple tunnel 13 of the breast cup 2 may have a largest diameter greater than 25 mm (e.g., greater than 50 mm, 75 mm, 100 mm, 125 mm, 150 mm, 175 mm, 200 mm, 225 mm, or 250 mm).

The breast cup 2, the breast shield 19, and/or the housing may include a gel such that it allows better conformance to the breast. In some embodiments, the breast cup 2 conforms to the breast and supports the weight of the breast cup 2 and fluid conduits extending from the breast cup 2 without additional adhesives, gels, straps, or bras. In some embodiments, the breast cup 2 secures to a garment, such as a bra or shirt.

The breast cup 2, the breast shield 19, and/or the housing may include a food contact substance. Additionally, or alternatively, the breast cup 2, the breast shield 19, and/or the housing may include an infant grade material. The breast cup 2, the breast shield 19, and/or the housing may include a flexible material, e.g., formed from a polymeric material such as silicone. The breast cup 2, the breast shield 19, and/or the housing may include a rigid material. The breast cup 2, the breast shield 19, and/or the housing may include PET, PVC, PTFE, polyethylene, HDPE, LDPE, copolyester (e.g., Tritan EX401, PP, polystyrene, a thermoplastic elastomer, thermoplastic polyurethane, PC, nylon, PVDF, or silicone. In some embodiments, breast cup 2, the breast shield 19, and/or the housing can include a fabric (e.g., polyester, cotton, linen, satin, organdy, rayon, taffeta, broad cloth, poplin, velour, gauze, canvas, shirting, muslin, tweed, georgette, crepe, wool, twill, gabardine, denim, or drill).

In some embodiments, the breast cup 2 may be in fluidic communication with the pressure source 9, such that milk is pumped from the breast to the reservoir 3.

In some embodiments, the breast cup 2 may have a second fluidic conduit providing fluidic communication between the breast cup 2 and the pressure source 9. In some embodiments, the breast cup 2 may have a first fluidic conduit providing fluidic communication between the breast cup 2 and a reservoir 3 or manifold 29. In some embodiments the first fluidic conduit is releasably connected to the breast cup 2 via a first outlet on the breast cup 2, e.g., from the milk chamber. In some embodiments, the second fluidic conduit is releasably connected to the breast cup 2 via an inlet to the breast cup 2, e.g., in the pressure chamber. In some embodiments, the first and second fluidic conduits are releasably connected to the breast cup 2 via a connector, e.g., wherein the inlet and outlet to which the first fluidic conduit and the second fluidic conduit connect are not disposed adjacent to each other. In some embodiments, the connector further comprises a half-moon shape.

In some embodiments, the breast cup 2 allows for the extension of the nipple when negative pressure is supplied, e.g., by the pressure source 9 e.g., at a first pressure, in order to express milk. When positive pressure is supplied by a control valve 10 and/or a positive pressure source, e.g., at a second pressure, the nipple contracts, and reduces or stops milk flow until a higher negative pressure is supplied.

In some embodiments, the interior of the breast cup 2, e.g., the interior of the breast shield 19, includes an open area, e.g., the nipple tunnel 13, dimensioned to allow for clearance and space in front of the nipple to permit milk to exit the nipple even when the nipple is pulled forward by suction.

In some embodiments, the modular breast pump system 1 includes two breast cups 2, e.g., one for each breast. In embodiments in which the modular breast pump system includes two breast cups 2, the two breast cups 2 can each include a housing, a breast shield 19, and a diaphragm 18. A user can use both of the breast cups 2 at the same time, or just one of the two at any given time.

One or more components of the breast cup may be clear to allow for visualization of the nipple, e.g., for alignment, during placement. The breast cup may also include an opaque cover or flat to make the nipple not visible after placement of the breast cup. The breast cup may also include a lens to magnify the nipple to aid in alignment.

### Breast Shield

The breast cup 2 can include a breast shield 19, e.g., as shown in FIGS. 13A-13D. The breast shield 19 contacts the breast of the user and can provide an airtight seal during breast pumping, such that milk does not leak from the breast cup 2. The breast shield 19 is configured to conform to the breast comfortably for frequent and/or long periods of wear, such that the breast is not irritated. The breast shield 19 can include a wide portion and a nipple tunnel, in which the wide portion initially contacts the breast, and the nipple tunnel is configured to receive the nipple. For example, the breast shield 19 can conform to the breast and provide space for the nipple, e.g., in the nipple tunnel.

In some embodiments, the nipple tunnel has a length from about 5 mm to about 75 mm (e.g., about 5 mm to about 10 mm, about 5 mm to about 20 mm, about 5 mm to about 30 mm, about 5 mm to about 40 mm, about 5 mm to about 50 mm, about 10 mm to about 20 mm, about 10 mm to about 30 mm, about 10 mm to about 40 mm, about 10 mm to about 50 mm, about 10 mm to about 60 mm, about 10 mm to about 70 mm, about 25 mm to about 50 mm, about 25 mm to about 75 mm, about 50 mm to about 75 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, 35 mm, about 40 mm, about 45 mm, about 50 mm, about 55 mm, about 60 mm, about 65 mm, about 70 mm, or about 75 mm).

In some embodiments, the nipple tunnel has a minimum diameter from about 10 mm to about 130 mm (e.g., about 10 mm to about 20 mm, about 10 mm to about 30 mm, about 10 mm to about 40 mm, about 10 mm to about 50 mm, about 10 mm to about 60 mm, about 10 mm to about 70 mm, about 10 mm to about 100 mm, about 25 mm to about 50 mm, about 25 mm to about 75 mm, about 30 mm to about 130 mm, about 50 mm to about 75 mm, about 50 mm to about 130 mm, about 70 mm to about 130 mm, about 100 mm to about 130 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, 35 mm, about 40 mm, about 45 mm, about 50 mm, about 55 mm, about 60 mm, about 65 mm, about 70 mm, about 75 mm, about 80 mm, about 90 mm, about 100 mm, about 110 mm, about 120 mm, about 125 mm, or about 130 mm). In some embodiments, the diameter of the nipple tunnel is between about 20 and about 40 mm.

In some embodiments, the nipple tunnel has a maximum diameter from about 10 mm to about 130 mm (e.g., about 10 mm to about 20 mm, about 10 mm to about 30 mm, about 10 mm to about 40 mm, about 10 mm to about 50 mm, about 10 mm to about 60 mm, about 10 mm to about 70 mm, about 10 mm to about 100 mm, about 25 mm to about 50 mm, about 30 mm to about 130 mm, about 25 mm to about 75 mm, from about 50 mm to about 75 mm, about 50 mm to about 130 mm, about 70 mm to about 130 mm, about 100 mm to about 130 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, 35 mm, about 40 mm, about 45 mm, about 50 mm, about 55 mm, about 60 mm, about 65 mm, about 70 mm, about 75 mm, about 80 mm, about 90 mm, about 100 mm, about 110 mm, about 120 mm, about 125 mm, or about 130 mm).

In some embodiments, the diameter of the nipple tunnel is between about 11 and 30 mm.

The breast shield 19 can be in a shape for improved conformance to the breast, e.g., a funnel, a cone, or a dome. The breast shield 19 can be rounded or toroidal, e.g., forming a ring.

The breast shield 19 can include an inlet to the milk chamber 21, such that milk may enter the milk chamber 21 once expressed from the nipple. In some embodiments, the diaphragm 18 releasably contacts the inlet, e.g., at a sealing point 23, providing a seal when negative pressure is not applied to the pressure chamber. Thus, in some embodiments, the diaphragm 18 releasably seals the inlet. The releasable sealing of the inlet to the milk chamber 21 is shown in FIG. 4.

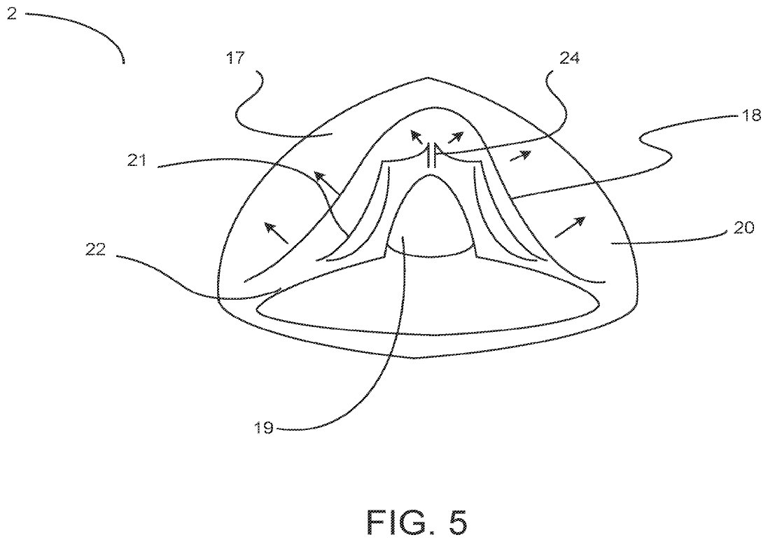

In some embodiments, the inlet includes a valve, e.g., an umbrella valve 24, e.g., as shown in FIG. 5, or a non-drip valve. In some embodiments, the valve is a mechanical valve, an umbrella valve 24, a butterfly valve, a disk valve, or a duckbill valve.

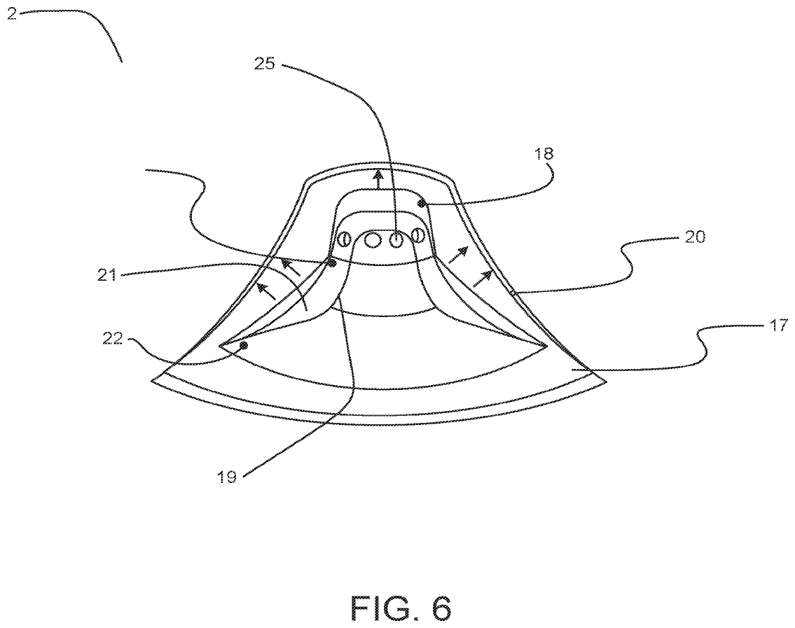

In some embodiments, the inlet includes at least one orifice 25, e.g., as shown in FIG. 6. In some embodiments, the inlet includes a plurality of orifices 25. In some embodiments, the plurality of orifices 25 are circumferentially arranged at the nipple tunnel of the breast shield 19, e.g., as shown in FIG. 6. In some embodiments, the diaphragm 18 releasably contacts at least one of the orifices 25, e.g., at sealing point 23, providing a seal when negative pressure is not applied to the pressure chamber 20. In some embodiments, the diaphragm 18 contacts the plurality of orifices 25. Thus, in some embodiments, the diaphragm 18 releasably seals at least one orifice, e.g., at sealing point 23. In some embodiments, the diaphragm 18 seals the plurality of orifices 25, e.g., at sealing point 23. In some embodiments, the diaphragm 18 releasably contacts the breast cup 19 at a point as to separate the at least one orifice 25 from the milk chamber 21.

In some embodiments, the orifice 25 can have a diameter from about 0.5 mm to about 10 mm (e.g., about 0.5 mm to about 1 mm, about 0.5 mm to about 1.5 mm, about 0.5 mm to about 2 mm, about 0.5 mm to about 5 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 3 mm to about 7 mm, about 5 mm to about 10 mm, about 8 mm to about 10 mm, about 1 mm, about 2 mm, about 3 mm, about 4 mm, about 5 mm, about 6 mm, about 7 mm, about 8 mm, about 9 mm, or about 10 mm).

In some embodiments, the diaphragm 18 is not configured to contact the inlet, e.g., at sealing point 23. In some embodiments, the breast cup 2, e.g., the breast shield 19 includes a fitting which may seal the inlet from the milk chamber 21. In some embodiments, the fitting is a plug or a cover.

The wide portion of the breast shield 19 can seal to the breast, while the nipple tunnel provides room for the nipple to be expanded to express milk. In some embodiments, the wide portion of the breast shield 19 seals to the breast, e.g., around the base of nipple. In some embodiments, the breast shield 19 is sealed to the breast before a breast pumping session commences.

In some embodiments, a positive or negative pressure may be applied to the breast cup 2 prior to a breast pumping session to contract the breast shield 19, e.g., the wide portion of the breast shield 19, such that the breast shield 19 may be sealed to the breast. Following a breast pumping session, a positive or negative pressure may be applied to the breast cup 2 such that the breast cup 2, e.g., the wide portion of the breast shield 19, may be unsealed from the breast. In some embodiments, the edge of the wide portion of the breast shield 19 solely contacts the breast before application of negative pressure. Following application of negative pressure to the breast cup 2, substantially all of the wide portion of the breast shield 19 may contact the breast.

Negative pressure can be applied to the breast cup 2 for sealing of the breast cup 2 to the breast from the pressure source 9, or from force applied manually from the user to the breast cup 2, e.g., to the housing. For example, the user may push on the breast cup 2, e.g., the housing, to provide negative pressure to the breast shield 19 and seal the breast cup 2 to the breast. Positive pressure can be applied to the breast cup 2 to unseal the breast cup 2 from the breast from the positive pressure source, from a valve to the ambient atmosphere, from a slow leak, or from force manually applied from the user to the breast cup 2. For example, a user may pull on the breast cup 2, e.g., the housing, to provide positive pressure to the breast shield 19 and unseal the breast cup 2 from the breast. In some embodiments, the breast cup 2 includes an air pocket, to which negative or positive pressure can be applied to seal or unseal the breast cup 2 to the breast.

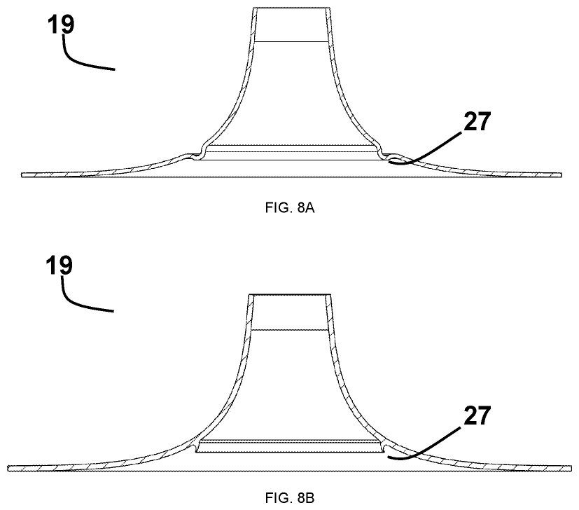

The inside of the breast shield 19 can include at least one protrusion 27 to improve sealing of the breast shield 19 to the breast. FIG. 8A and FIG. 8B show embodiments of the breast shield 19 having protrusions 27. In some embodiments, the protrusion 27 is a toroidal ridge, see, e.g., FIG. 8A. In some embodiments, the protrusion 27 is a curved toroidal ridge, see, e.g., FIG. 8B. In some embodiments, the outer surface of the breast shield 19 includes an indent to form the protrusion 27 on the inner surface of the breast shield 19, see, e.g., FIG. 8A.

Sealing of the breast shield 19 to the breast may keep milk from leaking around the breast and improve security of the breast shield 19 to the breast during breast pumping. Unsealing of the breast shield 19 from the breast when a breast pumping session is not ongoing, e.g., to allow air flow, may improve comfort, reduce skin temperature, and reduce sweat build-up. Furthermore, expansion and contraction of breast shield 19 allows for the breast shield 19 to fit breasts of a plurality of sizes, e.g., AA cup, A cup, B cup, C cup, D cup, DD cup, DDD cup, E cup, F cup, etc.

The ability to expand and contract the nipple tunnel of the breast shield 19, mechanically or using pressure, may be advantageous in setting up the modular breast pump system 1 for breast pumping. In some embodiments, the internal diameter of the nipple tunnel of breast shield 19 is reduced slowly while moving the breast shield 19 into position, such that the user may be able to center the breast shield 19 on their nipple via touch and feel without the need for line of sight. In some embodiments, the housing and diaphragm 18 may be or may include a clear material, such that the user may additionally use line of sight to center the breast shield 19 on their nipple.

In some embodiments, the breast shield 19 and/or the housing include ridges, bumps, and/or dimples. In some embodiments, an internal surface of the breast shield 19 and/or the housing include ridges, bumps, or dimples. Ridges, bumps, and/or dimples may aid in aligning the breast cup 2 to the breast, adherence of the breast cup 2 to skin, reduction of heat build-up, and may provide further comfort when a user is wearing the breast cup. In some embodiments, the ridges, bumps, and/or dimples include foam.

In some embodiments, the ridge includes a straight ridge, a curved ridge, a zig-zag ridge, an undulating ridge, a spiral ridge, a continuous ridge, or a broken ridge. The breast shield 19 can include a plurality of ridges. In some embodiments, the ridges are from about 1 mm to about 100 mm apart (e.g., about 1 mm to about 2 mm, about 1 mm to about 5 mm, about 1 mm to about 10 mm, about 1 mm to about 25 mm, about 1 mm to about 50 mm, about 5 mm to about 10 mm, about 10 mm to about 25 mm, about 10 mm to about 50 mm, about 25 mm to about 75 mm, about 50 mm to about 100 mm, about 1 mm, about 5 mm, about 10 mm, about 15 mm, about 20 mm, about 25 mm, about 30 mm, about 40 mm, about 50 mm, about 60 mm, about 70 mm, about 75 mm, about 80 mm, about 90 mm, or about 100 mm).

Air may flow between the ridges, bumps, and/or dimples where the surface of the breast shield 19 does not contact the breast. In some embodiments, the ridges, bumps, and/or dimples extend from an edge of the breast shield 19 to an inner point of the inner surface of the breast shield 19. In this regard, the ridges, bumps, and/or dimples may form an air passage from the ambient atmosphere to the inner surface of the breast shield 19. Thus, in some embodiments, the breast shield 19 includes a fluid conduit, wherein the fluid conduit may be defined by the (i) ridges, bumps, and/dimples, (ii) the skin of the breast, and (iii) the inner surface of the breast shield 19. The fluid conduit may create a chimney effect resulting in the advantage of improved air circulation in the breast shield 19.

Alternatively, or additionally, the breast cup 2 may be manually pressed in order to expel air from the breast shield 19. Following release of the breast cup 2, fresh air may flow back into the breast shield 19. In some embodiments, this method of circulating air in the breast shield 19 may be accomplished before or following a breast pumping session. For example, a spiral ridge on the inner surface of the breast shield 19 may form a fluid conduit with the skin of the breast. When the breast cup 2 is pressed, air inside the breast shield 19 may be transported around the spiral to the ambient atmosphere. When the pressure removed from the breast cup 2, fresh air may be transported around the spiral back into the breast shield 19.

Ridges, bumps, and/or dimples may also reduce the peeling effect when removing the breast cup 2. A reduction in peeling effect can improve comfort, especially for those users with sensitive skin.

The breast shield 19 may include a material which becomes soft by heating and further conforms to the geometry of the body. The breast shield 19 can include one or more formable chambers, which aid in conforming the breast shield 19 to the breast of the user. In some embodiments, the formable chambers include a fluid, e.g., a gas or liquid. In some embodiments, the breast shield 19 includes flexible, gel-like materials such as cross-linked silicone. In some embodiments, the breast shield 19 includes one or more thixotropic fluids.

In some embodiments, breast shield 19 includes a material which may store heat for improved comfort. In some embodiments, the breast shield 19 includes a material which may expel heat into the ambient environment. In some embodiments, the breast shield 19 includes regions of material which may store heat, and regions of material which may expel heat. In some embodiments, the regions of material which may expel heat include regions having ridges, bumps, and/or dimples.

The breast shield 19 can include a food contact substance. Alternatively, or additionally, the breast shield 19 can include an infant grade material. The breast shield 19 can include polyvinyl chloride (PVC), polyethylene, PP, polystyrene, a thermoplastic elastomer, thermoplastic polyurethane, PC, nylon, polyvinylidene fluoride (PVDF), or silicone. The breast shield 19 can include a flexible material, e.g., formed from a polymeric material such as silicone. The breast shield 19 can include a rigid material. In some embodiments, the breast shield 19 includes a water-resistant or a sweat-resistant material.

In some embodiments, two human breasts are contacted with two breast shields 19 to allow pumping milk from both breasts.

### Housing

The breast cup 2 may include a housing including an upper housing 17 and a lower housing or milk chamber wall 26. The upper housing 17 may be disposed on the exterior of breast cup 2, e.g., around the breast cup 2, e.g., be outward facing. The upper housing 17 may form a portion of the outer surface of the pressure chamber 20 or be a separate component.

In some embodiments, the housing may be disposed around the wide portion 8 of the breast shield 19, e.g., the first point of contact with the breast. The housing may conform to the breast. The housing may include a gel such that it conforms to the breast. In some embodiments, the housing conforms to the breast and supports the weight of the breast cup 2, the housing, and fluid conduits 5 extending from the breast cup 2 without additional adhesives, gels, straps, or bras. In some embodiments, the housing does not conform to the breast. The housing may secure to a garment, such as a bra or shirt.

The housing may serve a plurality of additional purposes including improved conformance of the breast cup 2 to the breast, improved comfort of the modular breast pump system 1, releasable attachment of the breast cup 2 to a garment, and/or blocking the breast from view while using the modular breast pump system 1.

The housing can include a food contact substance. In some embodiments, the housing can include a flexible material, e.g., formed from a polymeric material such as silicone. In some embodiments, the housing can include a rigid material. In some embodiments, the housing can include a fabric (e.g., polyester, cotton, linen, satin, organdy, rayon, taffeta, broad cloth, poplin, velour, gauze, canvas, shirting, muslin, tweed, georgette, crepe, wool, twill, gabardine, denim, or drill). The housing can include polyvinyl chloride (PVC), PTFE (e.g., Teflon), polyethylene, HDPE, LDPE, copolyester (e.g., Tritan EX401, PP, polystyrene, a thermoplastic elastomer, thermoplastic polyurethane, PC, nylon, polyvinylidene fluoride (PVDF), or silicone. In some embodiments, the housing can include a clear material.

### Diaphragm

The breast cup 2 may include a diaphragm 18. The diaphragm 18 may separate the pressure chamber 20 from the milk chamber 21.

The diaphragm 18 is configured to deform, i.e., expand, contract, or balloon, under pressure. Upon application of negative pressure to the pressure chamber 21, the diaphragm 18 may deform away from the inlet to the milk chamber 21, opening the inlet to the milk chamber 21. The breast cup 2 including the diaphragm 18 is advantageous in that the configuration may aid in providing fluid flow under pressure, as opposed to exclusively relying on gravity, such that a subject may breast pump in a reclined position.

In some embodiments, the diaphragm 18 includes a flexible material that may stretch upon depressurization and pressurization of the pressure chamber. Alternatively, the diaphragm 18 includes a folded, or otherwise shaped, material, which may unfold and refold upon depressurization and pressurization of pressure chamber 20. In some embodiments, the diaphragm 18 includes from 1 to 10 folds (e.g., 1 to 2, 1 to 3, 1 to 4, 1 to 5, 1 to 6, 1 to 7, 1 to 8, 1 to 9, 2 to 3, 2 to 4, 2 to 5, 2 to 6, 2 to 8, 3 to 4, 3 to 5, 3 to 7, 4 to 5, 4 to 6, 4 to 10, 5 to 8, 5 to 10, 8 to 10, 1, 2, 3, 4, 5, 6, 7, 8, 9, or 10). In some embodiments, the folded, or otherwise shaped, material is toroidal. In some embodiments, the folded, or otherwise shaped, material may unfold in either direction. In other embodiments, the folded, or otherwise shaped, material may unfold only towards the pressure chamber 20 or milk chamber 21. Flexible and/or folded diaphragms are advantageous in that they allow for expansion with smaller footprints.

In some embodiments, the diaphragm 18 includes a patterned material, such as including a plurality of indents or grooves. A diaphragm including a patterned material may be advantageous in optimizing interaction with the pressure chamber 20 and/or the milk chamber 21. In some embodiments, a first portion of the diaphragm 18 includes a plurality of indents and/or grooves. In some embodiments, the first portion of the diaphragm 18, which includes the plurality of indents and/or grooves, can deform more than a second portion of the diaphragm 18 which does not include a plurality of indents and/or grooves.

In some embodiments, the diaphragm 18 includes a material having a Shore hardness from A10 to A80 (e.g., A10, A20, A30, A40, A50, A60, A70, or A80). In some embodiments, the diaphragm 18 includes a material having a Shore hardness from D10 to D80 (e.g., D10, D20, D30, D40, D50, D60, D70, or D80).

In some embodiments, the diaphragm 18 includes a thickness from about 0.5 mm to about 10 mm (e.g., about 0.5 mm to about 1 mm, about 0.5 mm to about 1.5 mm, about 0.5 mm to about 2 mm, about 0.5 mm to about 2.5 mm, about 0.5 mm to about 5 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 5 mm to about 10 mm, about 0.5 mm, about 1 mm, about 1.5 mm, about 2 mm, about 2.5 mm, about 3 mm, about 4 mm, about 5 mm, about 6 mm, about 7 mm, about 8 mm, about 9 mm, or about 10 mm). The diaphragm may have a non-uniform thickness, e.g., with a thicker portion to seal an inlet or at the edge to seal to other components.

In some embodiments, the diaphragm 18 is configured to seal the inlet to the milk chamber 21 at sealing point 23. Sealing between the diaphragm 18 and the inlet may be improved through optimization of the Shore hardness, rigidity, thickness, material, surface conditions, and shape of the diaphragm 18.

In some embodiments, the diaphragm 18 varies in thickness and/or Shore hardness. The diaphragm 18 may include a higher thickness and/or higher Shore hardness in the portion of the diaphragm 18 which overlaps with the inlet of the milk chamber 21, e.g., at sealing point 23. In some embodiments, the diaphragm 18 includes a first thickness from about 0.5 mm to about 10 mm (e.g., about 0.5 mm to about 1 mm, about 0.5 mm to about 1.5 mm, about 0.5 mm to about 2 mm, about 0.5 mm to about 2.5 mm, about 0.5 mm to about 5 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 1 mm to about 12 mm, about 1 mm to about 15 mm, about 5 mm to about 10 mm, about 5 mm to about 15 mm, about 5 mm to about 20 mm, about 10 mm to about 15 mm, about 10 mm to about 20 mm, about 15 mm to about 20 mm, about 0.5 mm, about 1 mm, about 1.5 mm, about 2 mm, about 2.5 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm), and a second thickness from about 0.5 mm to about 10 mm (e.g., about 0.5 mm to about 1 mm, about 0.5 mm to about 1.5 mm, about 0.5 mm to about 2 mm, about 0.5 mm to about 2.5 mm, about 0.5 mm to about 5 mm, about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 1 mm to about 12 mm, about 1 mm to about 15 mm, about 5 mm to about 10 mm, about 5 mm to about 15 mm, about 5 mm to about 20 mm, about 10 mm to about 15 mm, about 10 mm to about 20 mm, about 15 mm to about 20 mm, about 0.5 mm, about 1 mm, about 1.5 mm, about 2 mm, about 2.5 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm), in which diaphragm 18 includes the second thickness where diaphragm 18 contacts the inlet, e.g., at sealing point 23. In some embodiments, the diaphragm 18 includes a material having a first Shore hardness from A10 to A80 (e.g., A10, A20, A30, A40, A50, A60, A70, or A80) and a second Shore hardness, different from the first, from A10 to A80 (e.g., A10, A20, A30, A40, A50, A60, A70, or A80) or being rigid, in which the material includes the second Shore hardness where diaphragm 18 contacts the inlet, e.g., at sealing point 23. In this embodiment, the second Shore hardness may further range from D10 to D80 (e.g., D10, D20, D30, D40, D50, D60, D70, or D80) or be rigid.

In some embodiments, the diaphragm 18 includes a curved shape, such that the surface of the diaphragm 18 curves over and around the inlet to the milk chamber 21. In some embodiments, the diaphragm 18 includes a parabolic shape on the side of the diaphragm that contacts the inlet. In some embodiments, the diaphragm 18 may have a cup or cone shape.

In some embodiments, the diaphragm 18 includes a plurality of layers, e.g., 1, 2, 3, 4, 5, 6, 7, 8, 9, or 10. In some embodiments, the diaphragm 18 includes a plurality of layers in which the diaphragm 18 contacts the inlet, e.g., at sealing point 23. In some embodiments, a second layer is over molded over a first layer of the diaphragm 18. The first layer and/or second layer may be a flexible or rigid material. In some embodiments, the first layer is a rigid material, and the second layer is a flexible material. This embodiment may be advantageous in sealing the inlet, such that the rigid material maintains coverage of the entire inlet, while the flexible material aids in sealing the sealing point 23. The first layer may be a disk and/or may include a ring or lip to aid in sealing. Alternatively, the first layer may be a cup or cone shaped layer over molded or embedded into the diaphragm 18. In some embodiments, the cup or cone shaped layer is configured to cap the inlet to the milk chamber 21, e.g., at sealing point 23. A plurality of layers may aid decreasing deflection and improving sealing performance at the point where the diaphragm 18 contacts the inlet of the milk chamber, e.g., at sealing point 23. In some embodiments, all or a portion of the diaphragm may be clear. In some embodiments, a clear portion may be configured to seal the inlet to the milk chamber 21, e.g., at the sealing point, and may provide a visual line of sight from the outside of the diaphragm 18 to the nipple tunnel 13. In this embodiment, the clear portion may contain a lens, wherein the lens allows for magnification. A visual line of sight to the nipple chamber may aid the user in centering the breast shield 19 on the nipple during a pumping session.

In some embodiments, the diaphragm 18 is shaped to conform to the shape of the breast shield 19, e.g., upon compression of the milk chamber 21. In some embodiments, the diaphragm 18 is not configured to contact the inlet, e.g., at sealing point 23. In some embodiments, the fitting is a plug or a cover. In some embodiments, the diaphragm 18 is configured to actuate a valve 24.

The diaphragm 18 can include a food contact substance. The diaphragm 18 can include polyvinyl chloride (PVC), polyethylene, PP, polystyrene, a thermoplastic elastomer, thermoplastic polyurethane, PC, nylon, polyvinylidene fluoride (PVDF), or silicone. The diaphragm 18 can include a flexible material, e.g., formed from a polymeric material such as silicone. The diaphragm 18 can include a rigid material.

### Milk Chamber

The breast cup 2 can include the milk chamber 21, which is configured to temporarily hold a volume of milk that is expressed from a nipple. The milk chamber 21 includes an inlet and an outlet 22.

In some embodiments, the milk chamber 21 is reversibly attached to the breast cup 2. In some embodiments, the milk chamber 21 is reversibly attached to the breast cup 2, such that smaller or larger milk chambers 21 may be reversibly attached to the breast cup 2.

The milk chamber 21 can have a volume, nominal or expanded, from about 1 mL to about 150 mL (e.g., about 1 mL to about 10 mL, about 1 mL to about 25 mL, about 1 mL to about 50 mL, about 1 mL to about 75 mL, about 1 mL to about 100 mL, about 25 mL to about 50 mL, about 25 mL to about 100 mL, about 50 mL to about 150 mL, about 100 mL to about 150 mL, about 1 mL, about 5 mL, about 10 mL, about 15 mL, about 20 mL, about 25 mL, about 30 mL, about 40 mL, about 50 mL, about 60 mL, about 70 mL, about 75 mL, about 80 mL, about 90 mL, about 100 mL, about 125 mL, or about 150 mL). The milk chamber 21 can have a maximum volume, nominal or expanded, from about 1 mL to about 150 mL (e.g., about 1 mL to about 10 mL, about 1 mL to about 25 mL, about 1 mL to about 50 mL, about 1 mL to about 75 mL, about 1 mL to about 100 mL, about 25 mL to about 50 mL, about 25 mL to about 100 mL, about 50 mL to about 150 mL, about 100 mL to about 150 mL, about 1 mL, about 5 mL, about 10 mL, about 15 mL, about 20 mL, about 25 mL, about 30 mL, about 40 mL, about 50 mL, about 60 mL, about 70 mL, about 75 mL, about 80 mL, about 90 mL, about 100 mL, about 125 mL, or about 150 mL). In some embodiments, the milk chamber 21 has a nominal volume of less than about 10 mL (e.g., less than about 5 mL, less than about 4.5 mL, less than about 4 mL, less than about 3.5 mL, less than about 3 mL, less than about 2.5 mL, less than about 2 mL, less than about 1.5 mL, less than about 1 mL, less than about 0.5 mL, or less than about 0.1 mL). In some embodiments, the milk chamber has a nominal volume of about 30 mL.

In some embodiments, the diaphragm 18 seals the inlet to the milk chamber 21 before positive pressure, e.g., at ambient pressure, is applied to pressure chamber 20 and the milk chamber 21 is compressed, e.g., at sealing point 23. In some embodiments, the seal between the diaphragm 18 and the inlet to the milk chamber 21 has greater sealing pressure than that resistance of one directional valve on the outlet of the milk chamber 21.

As described herein, the milk chamber may be formed by the space between the breast shield and the diaphragm or between one or more additional layers between the diaphragm and the breast shield.

The milk chamber may include an outlet. The outlet may include a one directional valve, e.g., to prevent air or expressed milk from being drawn back into the milk chamber during milk expression. The valve opens to allow milk to flow out of the milk chamber. In some embodiments, the one directional valve is passively actuated by pressure changes in the milk chamber. The milk chamber may also include an inlet, e.g., as a vent or to allow additional fluids, e.g., cleaning fluids or positive pressure to enter.

### Milk Chamber Wall

The breast cup 2 may include a milk chamber wall 26.

While in some embodiments, the milk chamber 21 is defined between the diaphragm 18 and the breast shield 19, the milk chamber 21 can alternatively be defined between the diaphragm 18 and the milk chamber wall 26, e.g., as shown in FIG. 7. Thus, in some embodiments, the milk chamber wall 26 is disposed between the diaphragm 18 and the breast shield 19, thereby defining the milk chamber 21 between the diaphragm 18 and the milk chamber wall 26. The milk chamber wall is advantageous, in that it can provide additional structural stability to the milk chamber.

In some embodiments, the milk chamber wall 26 can include a curved shape or a cup-shape.

In some embodiments, the milk chamber wall 26 is removably attached to the breast cup 2. In some embodiments, the milk chamber wall 26 is snapped into the breast cup 2. In some embodiments, the milk chamber wall 26 can be inserted into the breast cup 2 to reduce the volume of the milk chamber 21.

In some embodiments, the milk chamber wall 26 includes an orifice for outlet 22.

In some embodiments, the milk chamber wall 26 can include polyethylene terephthalate (PET), polypropylene (PP), polyethylene, high-density polyethylene (HDPE), low-density polyethylene (LDPE), or polycarbonate (PC). The milk chamber wall may include a clear material.

### Pressure Chamber

The breast cup 2 can include the pressure chamber 20, which is configured to expand and compress the diaphragm 18 in order to express milk from a nipple.

In some embodiments, the pressure chamber 20 is reversibly attached to the breast cup 2.

The pressure chamber 20 can have a maximum volume from about 1 mL to about 150 mL (e.g., about 1 mL to about 10 mL, about 1 mL to about 15 mL, about 1 mL to about 20 mL, about 1 mL to about 25 mL, about 1 mL to about 50 mL, about 1 mL to about 75 mL, about 1 mL to about 100 mL, about 5 mL to about 10 mL, about 5 mL to about 15 mL, about 5 mL to about 20 mL, about 5 mL to about 25 mL, about 5 mL to about 50 mL, about 10 mL to about 15 mL, about 10 mL to about 20 mL, about 10 mL to about 25 mL, about 10 mL to about 30 mL, about 10 mL to about 50 mL, about 15 mL to about 20 mL, about 15 mL to about 25 mL, about 15 mL to about 30 mL, about 15 mL to about 50 mL, about 20 mL to about 25 mL, about 20 mL to about 30 mL, about 20 mL to about 50 mL, about 25 mL to about 50 mL, from about 25 mL to about 100 mL, from about 50 mL to about 150 mL, from about 100 mL to about 150 mL, about 1 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 25 mL, about 30 mL, about 40 mL, about 50 mL, about 60 mL, about 70 mL, about 75 mL, about 80 mL, about 90 mL, about 100 mL, about 125 mL, or about 150 mL). The pressure chamber may have a maximum volume of about 45 mL.

In some embodiments, the pressure chamber 20 limits the shape of the diaphragm 18 at the maximum deflection of the diaphragm 18, thus restricting the maximum negative pressure that can be applied to the nipple.

In some embodiments, upon application of positive pressure, the diaphragm 20 moves towards its nominal position and extends past it under pressure to conform to the shape of the milk chamber 21, thus transporting all liquid through the outlet 22.

In some embodiments, the pressure chamber 20 further includes a valve to the ambient atmosphere. In some embodiments, the valve is an inflation valve. In some embodiments, volume of the pressure chamber 20 may be set with the inflation valve. In some embodiments, the valve is a relief valve. The relief valve may be configured to release pressure from the pressure chamber 20 if the positive pressure exceeds a maximum positive pressure, or if the negative pressure exceeds a maximum negative pressure.

While in some embodiments, the volume of the pressure chamber 20 may be set with an inflation valve, the volume of the pressure chamber 20 may be set in a variety of manners. In some embodiments, the pressure chamber 20 is reversibly attached to the breast cup 2, such that smaller or larger pressure chambers 20 may be reversibly attached to the breast cup 2. In some embodiments, the breast cup includes a plug, which may be configured to be inserted into the pressure chamber 20 to reduce the volume. In some embodiments, the volume of the pressure chamber 20 may be adjusted manually by the user such that it sits further or closer to the chest of the user.

The pressure chamber 20 can be returned to ambient pressure following a breast pumping session.

The pressure chamber 20 includes one or more inlets to allow for changes in pressure. The pressure chamber or housing may include a limiter that controls the maximum volume of the pressure chamber, e.g., to determine the pressure limit. For example, the breast shield may include a bladder or other element that can be filled with air or other fluid, e.g., water, and disposed to reduce the volume available for expansion of the pressure chamber.

### Milk Distribution System

The United States Food and Drug Administration (FDA) advises against mixing freshly expressed breast milk with already cooled or frozen milk as it can rewarm the older stored milk. The FDA also advises against storing milk at room temperature for more than 4 hours. Thus, it is advantageous to have a modular breast pump system 1 that can provide for multiple breast pumping sessions without the removal of a breast cup 2, as well as divert milk to different reservoirs 3 from a plurality of breast pumping sessions.

The present disclosure provides a milk distribution system, e.g., a breast milk distribution system 28. The milk distribution system is advantageous in that it may facilitate repeated breast pumping without the removal of a breast cup 2 from a breast and/or without the replacement of a reservoir 3 from a breast pump system 1. The presently disclosed modular breast pump system 1 may include the milk distribution system 28, e.g., in the reservoir enclosure in the base module 7.

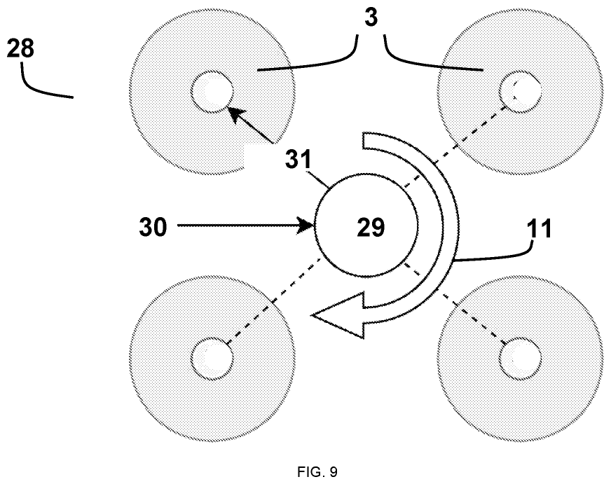

In some embodiments, the milk distribution system 28 can include a breast cup 2 and a manifold 29 comprising an inlet 30 and a plurality of outlets 31, e.g., as shown in FIG. 9. Each outlet 31 may provide fluidic communication to a different reservoir 3.

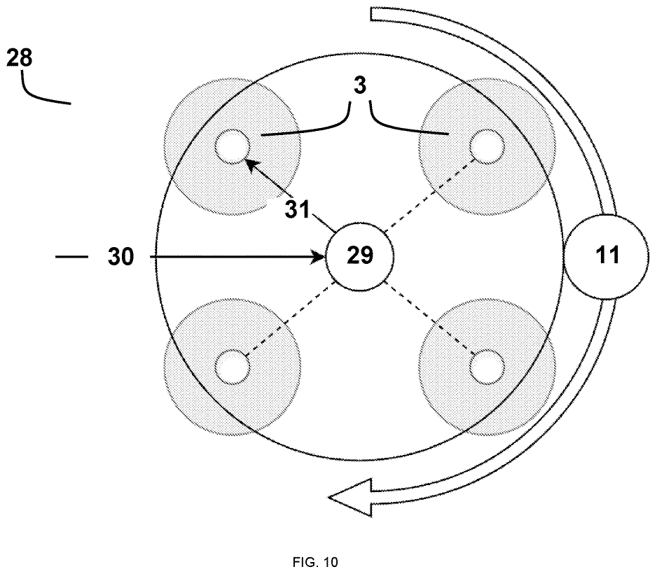

Alternatively, the manifold 29 can include an inlet 30 and an outlet 31, in which the outlet 31 is configured to provide fluidic communication to a plurality of reservoirs 3. In some embodiments, the milk distribution system 28 includes a manifold 29 in fluidic communication with the breast cup 2, wherein the manifold 29 includes an inlet 30 and an outlet 31 configured to move from a first position to a second position, e.g., as shown in FIG. 10.

In some embodiments, the milk distribution system 28 includes a plurality of reservoirs 3, e.g., 1, 2, 3, 4, 5, 6, 7, 8, 9, 10, 11, 12, 13, 14, 15, 16, 17, 18, 19, or 20. In some embodiments, the milk distribution system 28 can include a manifold 29 comprising an inlet 30 and a plurality of outlets 31, wherein each outlet 31 comprises a valve 24; and a plurality of reservoirs 3, wherein each of the plurality of reservoirs 3 is in fluidic communication with a different outlet 31.

In some embodiments, the milk distribution system 28 is configured to distribute milk from a plurality of breast pumping sessions based on a pre-programmed pumping schedule input by a user. In some embodiments, milk from a first pre-programmed breast pumping session of the pre-programmed pumping schedule is transported to a first reservoir 3, and milk from a second pre-programmed breast pumping session of the pre-programmed pumping schedule is transported to a second reservoir 3. Different pumping sessions may be spaced apart by time, e.g., 3-4 hours.

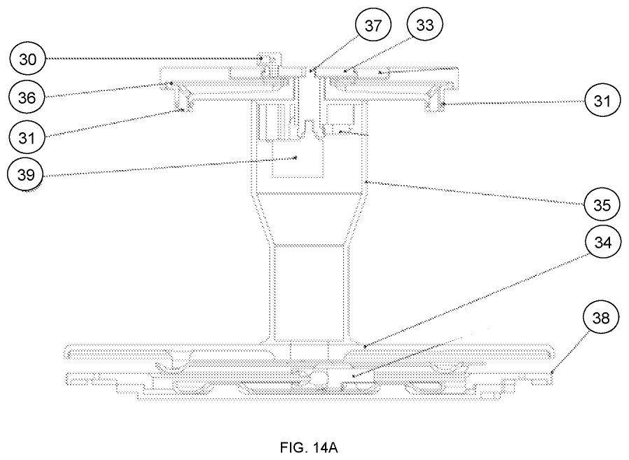

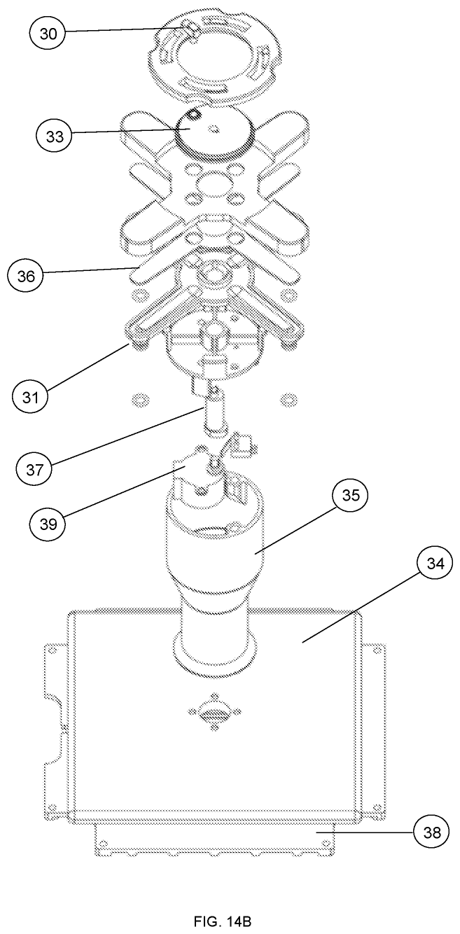

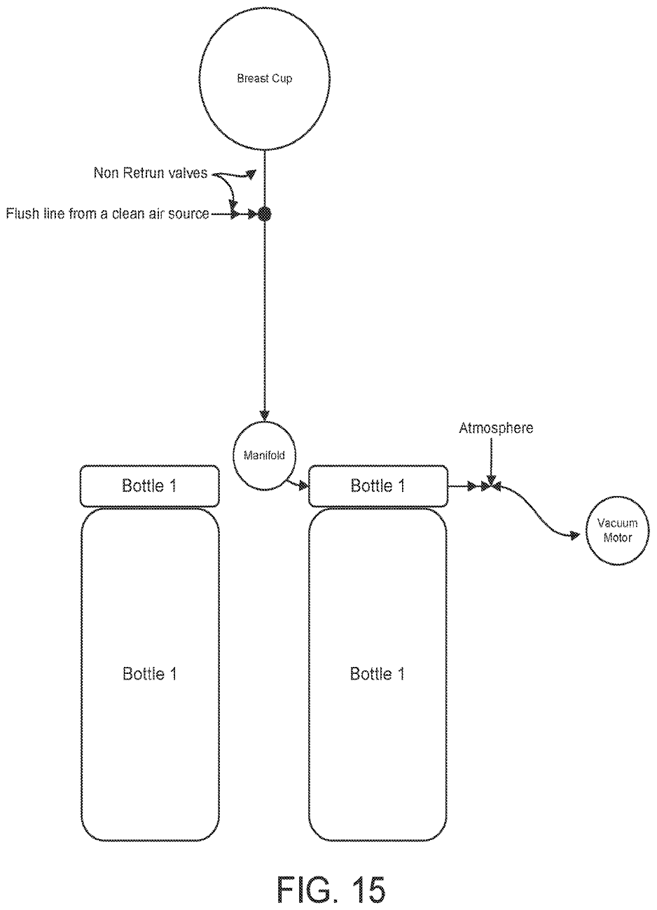

Pumping milk to the milk distribution system may occur by the action of the breast cup. Other pumping mechanisms may also be employed, including providing positive pressure to the milk chamber, e.g., by forced air or providing negative pressure to a fluid conduit between the breast cup and a reservoir. Negative pressure may be employed by a peristaltic pump or by air being pulled into the fluid conduit. These may also act to flush the conduit after a pumping session or after a set number of pumping session. For example, a flush line may connect to the fluid conduit, with both the flush line and the fluid conduit having one directional valves to prevent back flow. Negative pressure may be applied by reducing pressure in a reservoir, and a vent may be present to release pressure when desired, as reflected in the system shown in, e.g., FIG. 15.

### Manifold

The milk distribution system 28 can include a manifold. The manifold may include at least one inlet and at least one outlet. The manifold may also include at least one valve. In some embodiments, the manifold is disposed in the base module 7, e.g., in the reservoir enclosure.

In some embodiments, the manifold is disposed between the breast cup 2 and a plurality of reservoirs 3. In some embodiments, the manifold is disposed between two breast cups 2 and the plurality of reservoirs 3. Milk from different breast pumping sessions may be directed to different reservoirs 3, such as different breast pumping sessions in the same evening. In some embodiments, each reservoir 3 includes an inlet, wherein each inlet of the reservoir in fluidic communication with a different outlet of the manifold 29. In some embodiments, the inlet of the reservoir 3 is configured to open and close. Switching from a first, second, third, fourth, fifth, etc., reservoir can be manual or automatic.

In some embodiments, the manifold includes a substrate having a plurality of fluid conduits disposed therein. In some embodiments, the manifold includes a straight manifold, a right-angle manifold, a round manifold, a block manifold, a square manifold, a hex manifold, a wye manifold, or a rotating joint manifold. In some embodiments, the manifold includes a straight manifold or a right-angle manifold. In a straight manifold, fluid enters and exits the manifold in the same direction. In a right-angle manifold, fluid exits the manifold at a right angle from the direction the fluid entered the manifold.

In some embodiments, the manifold includes an inlet. In some embodiments, the manifold includes from 1 to 20 inlets (e.g., from 1 to 2, from 1 to 3, from 1 to 4, from 1 to 5, from 1 to 6, from 1 to 7, from 1 to 8, from 1 to 9, from 1 to 10, from 1 to 15, from 2 to 3, from 2 to 4, from 2 to 5, from 2 to 6, from 2 to 7, from 2 to 8, from 3 to 4, from 3 to 5, from 3 to 6, from 3 to 7, from 3 to 8, from 4 to 5, from 4 to 6, from 4 to 8, from 4 to 10, from 5 to 6, from 5 to 7, from 5 to 8, from 5 to 10, from 6 to 8, from 6 to 10, from 6 to 12, from 8 to 10, from 8 to 12, from 10 to 12, from 10 to 15, from 10 to 20, from 12 to 16, from 15 to 20, 1, 2, 3, 4, 5, 6, 7, 8, 9, 10, 11, 12, 13, 14, 15, 16, 17, 18, 19 or 20).

In some embodiments, the inlet is threaded. In some embodiments, the inlet includes internal threads and/or external threads. In some embodiments, the inlet includes a fluidic fitting. In some embodiments, the inlet includes a Luer fitting, wherein the Luer fitting may releasably connect the inlet and a fluid conduit 5.

In some embodiments, the manifold includes an outlet. In some embodiments, the manifold includes from 1 to 20 outlets (e.g., from 1 to 2, from 1 to 3, from 1 to 4, from 1 to 5, from 1 to 6, from 1 to 7, from 1 to 8, from 1 to 9, from 1 to 10, from 1 to 15, from 2 to 3, from 2 to 4, from 2 to 5, from 2 to 6, from 2 to 7, from 2 to 8, from 3 to 4, from 3 to 5, from 3 to 6, from 3 to 7, from 3 to 8, from 4 to 5, from 4 to 6, from 4 to 8, from 4 to 10, from 5 to 6, from 5 to 7, from 5 to 8, from 5 to 10, from 6 to 8, from 6 to 10, from 6 to 12, from 8 to 10, from 8 to 12, from 10 to 12, from 10 to 15, from 10 to 20, from 12 to 16, from 15 to 20, 1, 2, 3, 4, 5, 6, 7, 8, 9, 10, 11, 12, 13, 14, 15, 16, 17, 18, 19 or 20). In some embodiments, the milk distribution system 28 further includes a plurality of reservoirs 3, wherein each of the plurality of reservoirs 3 is configured to be in fluidic communication with a different outlet 31 of the manifold 29. In some embodiments, the outlet is threaded. In some embodiments, the outlet includes internal threads and/or external threads. In some embodiments, the outlet includes a Luer lock connection, wherein the Luer lock connection may releasably connect the outlet and a fluid conduit 5. The outlet may include an O-ring or other gasket to seal to an inlet of a reservoir.

In some embodiments, one inlet branches into from 2 to 20 outlets (e.g., from 2 to 3, from 2 to 4, from 2 to 5, from 2 to 6, from 2 to 7, from 2 to 8, from 3 to 4, from 3 to 5, from 3 to 6, from 3 to 7, from 3 to 8, from 4 to 5, from 4 to 6, from 4 to 8, from 4 to 10, from 5 to 6, from 5 to 7, from 5 to 8, from 5 to 10, from 6 to 8, from 6 to 10, from 6 to 12, from 8 to 10, from 8 to 12, from 10 to 12, from 10 to 15, from 10 to 20, from 12 to 16, or from 15 to 20, or 2, 3, 4, 5, 6, 7, 8, 9, 10, 11, 12, 13, 14, 15, 16, 17, 18, 19 or 20).

In some embodiments, the manifold includes at least one valve. In some embodiments, at least one inlet includes a valve. In some embodiments, at least one outlet includes a valve. In some embodiments, each outlet includes a valve. In some embodiments, the valve includes a ball valve, a batch dispensing valve, a butterfly valve, a diaphragm valve, a diverting valve, a gate valve, a pinch valve, a piston valve, a plug valve, a saddle valve, a solenoid valve, a stem valve, or a stop cock valve. In some embodiments, the valve includes at least one inlet and at least one outlet.

In some embodiments, the outlet of the valve is configured to move between a plurality of the outlets of the manifold. In some embodiments, the valve is a ball valve. In some embodiments, the ball of the ball valve includes at least one inlet and at least one outlet. In some embodiments, the ball may rotate to provide fluidic communication between outlet of the ball and a first outlet of the manifold and/or a second outlet of the manifold.

In some embodiments, the valve includes a solenoid valve. In some embodiments, each outlet of the manifold includes a solenoid valve. The milk distribution system 28 can include a drive unit configured to open and close the solenoid valve. In some embodiments, the solenoid valve includes a drive unit configured to open and close the solenoid valve. In some embodiments, the solenoid valve is a pinch valve.

In some embodiments, the breast milk distribution system 28 includes a drive unit 11 configured to open and close the valve 24. In some embodiments, the drive unit 11 is a stepper motor or a linear actuator. In some embodiments, the linear actuator is an electric linear actuator, a hydraulic linear actuator, or a pneumatic linear actuator. In some embodiments, the stepper motor 39 is attached to the manifold, wherein the stepper motor is configured to rotate the manifold 29.

The manifold 29 can be a rotary manifold, in which a plurality of outlets 31 is oriented circumferentially so as to line up with a plurality of reservoirs 3, wherein the manifold is configured to rotate and provide fluidic communication between the inlet 30 and each outlet 31 in sequence.

In some embodiments, the manifold 29 includes a moving, e.g., rotating, member and a stationary member. Each of the moving, e.g., rotating, member and stationary member can include a first end, a second end, and a body disposed therebetween. In some embodiments, each of the moving, e.g., rotating, member and stationary member include a first surface at the first end, and second surface at the second end. In some embodiments, the moving, e.g., rotating, member and/or the stationary member may include a channel disposed therein to transport milk and/or air. The body of each of the moving, e.g., rotating, member and/or the stationary member can have an outer surface.

In some embodiments, the moving, e.g., rotating, member is disposed within the stationary member. Alternatively, in some embodiments, the moving, e.g., rotating, member is disposed outside the stationary member.

In some embodiments, the moving, e.g., rotating, member and/or the stationary member includes an inlet 30 to the manifold 29. In some embodiments, the inlet 30 of the manifold is disposed on the first surface, second surface, or outer surface of the moving, e.g., rotating, member and/or the stationary member. In some embodiments, the moving, e.g., rotating, member and/or the stationary member includes at least one outlet 31 of the manifold 29. In some embodiments, at least one outlet 31 of the manifold is disposed on the first surface, second surface, or outer surface of the moving, e.g., rotating, member and/or the stationary member. In some embodiments, the outlets 31 of the manifold are axially or concentrically disposed in the outer surface of the moving, e.g., rotating, member or the stationary member. In some embodiments, the inlet 30 of the manifold 29 includes a longitudinal axis orthogonally arranged to a longitudinal axis of at least one of the outlets 31 of the manifold 29. In some embodiments, the inlet 30 of the manifold 29 includes a longitudinal axis orthogonally arranged to a longitudinal axis of each of the outlets 31 of the manifold 29.

In some embodiments, the moving, e.g., rotating, member and/or the stationary member include a fluid conduit 5 disposed therein, e.g., within the body. In some embodiments, the fluid conduit 5 is disposed in the body of the moving, e.g., rotating, member and/or the stationary member to provide fluidic communication between an inlet 30 of the manifold 29 and an outlet 31 of the manifold 29. In some embodiments, the fluid conduit 5 includes an outlet, and wherein the diameter of the outlet of the fluid conduit 5 includes the same diameter as the outlets 31 of the manifold 29. In alternative embodiments, the diameter of the outlet of the fluid conduit 5 is larger than the diameter of the outlets 31 of the manifold 29. In still other embodiments, the diameter of the outlet of the fluid conduit 5 is smaller than the diameter of the outlets 31 of the manifold 29. In some embodiments, the moving, e.g., rotating, member is configured to rotate and provide fluidic communication between the outlet of the fluid conduit 5 and the outlet 31 of the manifold 29, thus providing fluidic communication between the inlet 30 of the manifold and that outlet 31 of the manifold 29. In such an embodiment, the inlet 30 of the manifold 29 may or may not be in fluidic communication with other outlets 31 of the manifold.

In some embodiments, the manifold 29 includes (i) a moving, e.g., rotating, member including the inlet 30 of the manifold 29 and a fluid conduit, wherein the inlet 30 of the manifold 29 and the fluid conduit are in fluidic communication, and (ii) a stationary member including the plurality of outlets of the manifold 29; wherein the moving, e.g., rotating, member is configured to rotate and align the fluid conduit 5 with each of the plurality of outlets 31 of the manifold 29. In some embodiments, the moving, e.g., rotating, member is axially or concentrically arranged within the stationary member. In some embodiments, the outlets of the manifold 29 are radially disposed in the stationary member.

In some embodiments, the milk distribution system 28 includes a manifold 29 in fluidic communication with the breast cup 2, wherein the manifold 29 includes an inlet 30 and an outlet 31 configured to move from a first position to a second position. In some embodiments, each of the plurality of reservoirs 3 are configured to be in fluidic communication with the outlet of the manifold. In some embodiments, the outlet 31 of the manifold 29 is configured to be in fluidic communication with one reservoir at a time. In some embodiments, the milk distribution system 28 further includes a drive unit 11 configured to move the outlet 31. In some embodiments, the drive unit 11 is a stepper motor or a linear actuator. In some embodiments, the linear actuator is an electric linear actuator, a hydraulic linear actuator, or a pneumatic linear actuator. In some embodiments, the manifold 29 includes the stepper motor, and wherein the stepper motor is configured to radially move the outlet of the manifold 29.

In some embodiments, the manifold 29 includes an arm having the outlet 31 of the manifold, wherein the arm is configured to rotate and provide fluidic communication between the outlet 31 and each reservoir 3 individually. Thus, in some embodiments, the breast milk distribution system 28 further includes an arm, wherein the fluid conduit is disposed in and/or on the arm. In some embodiments, the arm includes an articulating arm. In some embodiments, the arm is hollow, and the fluid conduit 5 is disposed inside the arm. In some embodiments, the fluid conduit 5 is releasably connected to the arm.

In some embodiments, the manifold includes a stationary portion with two or more arms, e.g., four. Each arm has a channel connecting an inlet 30 and an outlet 31, which is configured to be in fluidic communication with a reservoir 3. For example, the outlets may have an O-ring or other seal to seal to the reservoirs. The arms may include a top plate and a bottom plate that are sealed with a gasket 36. The manifold may also include an alignment plate 33 that has an inlet. The alignment plate can be moved, e.g., rotated, to align its inlet with that of one of the arms of the manifold. The alignment plate inlet is in fluidic communication with a fluidic conduit from a breast cup. The manifold may include a motor 39, e.g., a stepper motor, with associated drive dog 37 to mate with the alignment plate, and a vertical body 35 to move the alignment plate and support the arms. The manifold may further include a weighing plate 34 comprising a weight sensor, e.g., a scale, and a stationary base 38 disposed at the bottom of the manifold. The reservoir 3 may rest on top of the weighing plate.

### Drive Unit and Control Unit

The modular breast pump system 1 and/or the milk distribution system 28 may include a drive unit 11, e.g., as shown in FIG. 10. The drive unit 11 provides power to the elements of the modular breast pump system 1.

In some embodiments, the drive unit 11 is a stepper motor or a linear actuator. In some embodiments, the linear actuator is an electric linear actuator, a hydraulic linear actuator, or a pneumatic linear actuator. In some embodiments, the drive unit is a stepper motor.

The drive unit 11 may be electrically connected to the pressure source 9 (e.g., the negative pressure source or the positive pressure source) and the control valve 10. The milk distribution system can include a drive unit 11, e.g., one configured to move at least one reservoir 3 or the manifold 29. In some embodiments, the stepper motor 39 is attached to the manifold 29, and the stepper motor is configured to move the manifold 29, e.g., the moving, e.g., rotating, member of the manifold 29.

In some embodiments, the modular breast pump system 1 and/or the milk distribution system 28 may include a control unit. The drive unit 11 may include a control unit. The control unit provides instructions to the drive unit 11, e.g., pre-programmed instructions or instructions provided by an external computer. The pre-programmed instructions or instructions provided by an external computer may be pumping instructions, heating instructions, cooling instructions, manifold instructions, time instructions, etc. For example, the control unit can be configured to (i) send a signal to the valve 24 to open and close or (ii) send a signal to the drive unit 11 to change the pressure in the pressure source 9, or to move the moveable, e.g., rotating, member.

### Fluid Conduit

The modular breast pump system 1 may include at least one fluid conduit 5, to provide movement of milk and other fluids, e.g., air. In some embodiments, the at least one fluid conduit 5 is flexible.

The fluid conduits 5 may include tubing. The fluid conduit 5 may include a food contact substance. The fluid conduit 5 may include an infant grade material. The fluid conduit 5 may include polyacetal, polyoxymethylene (POM), chlorinated polyvinyl chloride (CPVC), ethylene tetrafluoroethylene (ETFE), ethylene-vinyl acetate (EVA), fluorinated ethylene propylene (FEP), nylon, polyether ether ketone (PEEK), perfluoroalkoxy alkane (PFA), PC, polyethylene, PP, PTFE (e.g., Teflon), PVC, PVDF, thermoplastic elastomer (TPE), fluorosilicone, gum, latex, neoprene, polyurethane, rubber, rubber particles encapsulated in a PP matrix (e.g., Santoprene), or silicone. In some embodiments, the fluid conduits include an infant grade material.

In some embodiments, the fluid conduit 5 includes an internal diameter (ID) from about 0.5 mm to about 50 mm (e.g., about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 0.5 mm to about 5.5 mm, about 0.5 mm to about 6 mm, about 0.5 mm to about 6.5 mm, about 0.5 mm to about 7 mm, about 0.5 mm to about 7.5 mm, about 0.5 mm to about 10 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 10 mm, about 1 mm to about 25 mm, about 1 mm to about 30 mm, about 1 mm to about 40 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 3 mm to about 4 mm, about 3 mm to about 5 mm, about 4 mm to about 5 mm, about 4 mm to about 6 mm, about 5 mm to about 8 mm, about 5 mm to about 10 mm, about 5 mm to about 15 mm, about 5 mm to about 25 mm, about 5 mm to about 50 mm, about 10 mm to about 25 mm, about 20 mm to about 30 mm, about 25 mm to about 50 mm, about 30 mm to about 40 mm, about 30 mm to about 50 mm, or about 40 mm to about 50 mm).

In some embodiments, the fluid conduit 5 includes an outer diameter (OD) from about 0.5 mm to about 50 mm (e.g., about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 0.5 mm to about 5.5 mm, about 0.5 mm to about 6 mm, about 0.5 mm to about 6.5 mm, about 0.5 mm to about 7 mm, about 0.5 mm to about 7.5 mm, about 0.5 mm to about 10 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 10 mm, about 1 mm to about 25 mm, about 1 mm to about 30 mm, about 1 mm to about 40 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 3 mm to about 4 mm, about 3 mm to about 5 mm, about 4 mm to about 5 mm, about 4 mm to about 6 mm, about 5 mm to about 8 mm, about 5 mm to about 10 mm, about 5 mm to about 15 mm, about 5 mm to about 25 mm, about 5 mm to about 50 mm, about 10 mm to about 25 mm, about 20 mm to about 30 mm, about 25 mm to about 50 mm, about 30 mm to about 40 mm, about 30 mm to about 50 mm, or about 40 mm to about 50 mm).

The modular breast pump system 1 may be configured such that the user may roll during rest and/or sleep. The modular breast pump system may be configured such that the user may remove a reservoir from the reservoir enclosure without removing connection to a breast cup and/or the base and/or portable module 6. The modular breast pump system may be configured such that the user may remove the connection between a breast cup and the base and/or portable module 6 without removing the breast cup from the user. Currently available breast pump systems are not designed to allow for removal of the reservoir while leaving a breast cup in place.

The fluid conduit 5 may be selected to have a desired durometer to provide the preferred amount of flexibility for allowance of movement. For example, the fluid conduit 5 may have a durometer from about 25 Shore A scale and about 100 Shore D scale, including 25 A, 30 A, 35 A, 40 A, 45 A, 50 A, 55 A, 60 A, 65 A, 70 A, 75 A, 80 A, 85 A, 90 A, 95 A, 100 A, 10 D, 25 D, 30 D, 35 D, 40 D, 45 D, 50 D, 55 D, 60 D, 65 D, 70 D, 75 D, 80 D, 85 D, 90 D, 95 D, 100 D, or any durometer therebetween.

The fluid conduit 5 may be configured for ease of cleaning. For example, the modular breast pump system 1 may be run through with water to clean the fluid conduit 5.

In some embodiments, the fluid conduit 5 may be disposed in the center of the breast cup 2, e.g., in line with a nipple. In some embodiments, the fluid conduit 5 may be disposed at the base of breast cup 2, such that milk is expressed to the side. In some embodiments, the fluid conduit 5 may be disposed at the side of the breast cup 2.

The modular breast pump system 1 may include any number of fluid conduits 5, e.g., one, two, three, four, five, six, seven, eight, nine, ten, etc. A portion of fluid conduits 5 may provide air flow to the modular breast pump system 1, while other fluid conduits 5 transport milk.

In some embodiments, the modular breast pump system 1 includes a first fluid conduit 5 providing fluidic communication between the milk chamber 21 and the reservoir 3. In some embodiments, the modular breast pump system 1 includes a second fluid 5 conduit providing fluidic communication between the pressure source and the breast cup 2. In particular, in some embodiments, a second fluid conduit 5 provides fluidic communication between (a) the pressure source 9 and (b) the pressure chamber 20.

In some embodiments, the modular breast pump system 1 includes a first fluid conduit 5 providing fluidic communication between the breast cup 2 and the pressure source 9. In some embodiments, the first fluid conduit is releasably connected to the breast cup 2 and/or pressure source 9. The modular breast pump system 1 may include a second fluid conduit 5 providing fluidic communication between the pressure source 9 and the reservoir 3. In some embodiments, the second fluid conduit 5 is releasably connected to the pressure source 9 and/or reservoir 3. The modular breast pump system 1 may include a third fluid conduit 5 providing fluidic communication between the control valve 10 and the first fluid conduit 5. In some embodiments, the third fluid conduit 5 is releasably connected to the control valve 10 and/or first fluid conduit 5.

Additional fluid conduits 5 may transport milk from a second breast. In some embodiments, a first breast cup 2 is connected to a first fluid conduit 5, and a second breast cup 2 is connected to a second fluid conduit 5. In some embodiments, the first fluid conduit 5 and the second fluid conduit 5 merge into a third fluid conduit 5. In some embodiments, the third fluid conduit 5 connects to the manifold 29. Similarly, additional fluid conduits may connect a second breast cup 2 to the pressure source 32. These fluid conduits may also merge into a single conduit that connects to the pressure source.

In some embodiments, additional fluid conduits 5 may transport milk to additional reservoirs 3. In some embodiments, the milk from different breast pumping sessions is transported to the first reservoir 3 and/or second reservoir 3 through a fluid conduit 5. The milk transported to the first reservoir 3 and/or second reservoir 3 may be transported with the same or different fluid conduit 5, e.g., a first fluid conduit 5 and a second fluid conduit 5.

In some embodiments, the milk distribution system 28 includes a fluid conduit 5 providing fluidic communication between the breast cup 2 and the inlet 30 of the manifold 29. In some embodiments, the milk distribution system 28 further includes a fluid conduit 5 providing fluidic communication between the inlet of the manifold 30 and the outlet of the manifold 31. In some embodiments, the milk distribution system further includes a plurality of fluid conduits 5 providing fluidic communication between each of the outlets 31 of the manifold 29 and the plurality of reservoirs 3.

To facilitate better movement, the breast cup 2 and fluid conduits 5 may move with the user. Independent movement of the breast cup 2 and fluid conduits 5 allow the user to have a better range of motion, and further when the user moves the breast cup 2 does not experience force that might remove it from the breast during pumping. In some embodiments, the breast cup 2 includes a movement element. The movement element may be positioned around the outer edge of the breast cup. The movement element may be positioned at the nipple tunnel of the breast cup 2. In some embodiments, the first fluid conduit 5 is movably connected to the breast cup 2. In some embodiments, the first fluid conduit 5 is rotatable with respect to breast cup 2. In some embodiments, the first fluid conduit 5 is movably connected to the milk chamber 21. The movement element may be a swivel fitting or a rotating fitting. In some embodiments, the movement element includes a ball bearing.

The movement element may allow the fluid conduit 5 to rotate around the breast cup 2 from about 0° to about 360°, e.g., from about 0° to about 30°, from about 0° to about 60°, about 0° to about 90°, about 0° to about 120°, about 0° to about 150°, about 0° to about 180°, about 0° to about 210°, about 0° to about 240°, about 0° to about 300°, about 30° to about 120°, about 30° to about 180°, about 30° to about 240°, about 30° to about 360°, about 60° to about 120°, about 60° to about 180°, about 60° to about 240°, about 60° to about 360°, about 90° to about 180°, about 90° to about 360°, about 120° to about 240°, about 120° to about 360°, about 150° to about 240°, about 150° to about 360°, about 180° to about 240°, about 180° to about 360°, about 210° to about 360°, about 240° to about 360°, about 270° to about 360°, or about 300° to about 360°.

The movement element may allow the fluid conduit 5 to pivot from the center of the breast cup 2 from about 0° to about 90°, e.g., from about 0° to about 5°, about 0° to about 10°, about 0° to about 15°, about 0° to about 20°, about 0° to about 25°, about 0° to about 30°, about 0° to about 45°, about 0° to about 60°, about 0° to about 75°, about 5° to about 10°, about 5° to about 15°, about 5° to about 20°, about 5° to about 25°, about 5° to about 30°, about 5° to about 45°, about 5° to about 60°, about 5° to about 90°, about 10° to about 15°, about 10° to about 30°, about 10° to about 45°, about 10° to about 60°, about 10° to about 90°, about 30° to about 45°, about 30° to about 60°, about 30° to about 90°, about 45° to about 60°, about 45° to about 90°, or about 60° to about 90°.

A fluid conduit may have any appropriate connector at each end for connection to the breast cup 2, pump, or reservoir 3 or milk distribution system 28. Examples of connectors include Luer connectors, threaded connectors, and slip fit connectors.

When the portable module 6 is decoupled from the base module 7, one or more fluid conduits may be present between the pressure source in the portable module 6 and the breast cup or cups, and one or more fluid conduits may be present between the breast cup or cups and the reservoir. When the portable module 6 and base module 7 are mated, one or more fluid conduits may be present between the breast cup or cups and the pressure source in the portable module 6, and one or more fluid conduits may be present between the breast cup or cups and the reservoir or milk distribution system in the base module 7. When two breast cups are in use, each breast cup may have a separate fluid conduit to and from the pressure source and/or the reservoir. Alternatively, a single fluid conduit with two branches, e.g., with a Y or T connector, may be used to connect both breast cups to the pressure source and/or the reservoir or milk distribution system.

### Valves

The modular breast pump system 1 may include one or more valves. In some embodiments, the valve is a control valve, an open-close valve, a one directional valve, a relief valve, a quick-release valve, an inflation valve, or a slow-leak valve. In some embodiments, the valve is a mechanical valve, an inflation valve, an umbrella valve, a butterfly valve, a disk valve, a non-drip valve, a duckbill valve, a ball valve, a batch dispensing valve, a diaphragm valve, a gate valve, a diverting valve, a pinch valve, a piston valve, a plug valve, a saddle valve, a solenoid valve, a stem valve, a stop cock valve, or a three-way valve.

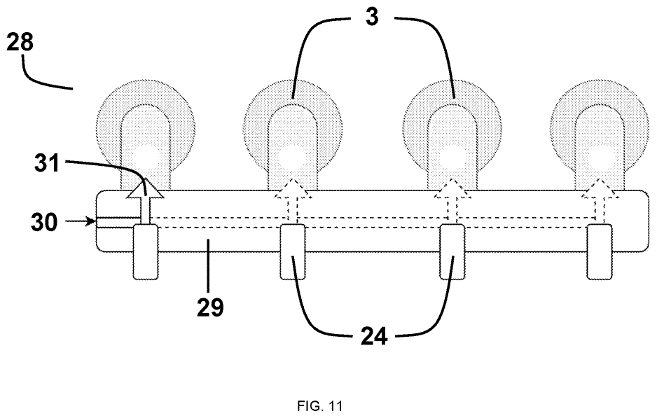

The milk distribution system 28 can include a valve 24. Such a valve 24 is advantageous in that it can provide selective fluidic communication to a plurality of reservoirs 3, e.g., as shown in FIG. 11. Thus, in some embodiments, the milk distribution system 28, can include a breast cup 2; a manifold 29 in fluidic communication with the breast cup 2, wherein the manifold 29 includes an inlet 30 and a plurality of outlets 31; and a valve 24 in fluidic communication with the manifold 29. In some embodiments, the breast milk distribution system 28 includes a plurality of valves 24, wherein each outlet 31 of the manifold 29 or the inlet of each reservoir 3 can include a valve 24. In some embodiments, the milk distribution system 28 is configured to close a valve 24 to a reservoir following a breast pumping session in which that reservoir 3 was filled.

The modular breast pump system 1 may include a control valve 10. The control valve 10 may be in fluidic communication with the breast cup 2 and the pressure source 9. In some embodiments, the fluid conduit 5 providing fluidic communication between the breast cup 2 and pressure source 9 includes the control valve 10. The control valve 10 may provide positive pressure to the breast cup 2 and/or the pressure source 9 in order to decrease the negative pressure provided by the pressure source 9, and reduce or stop milk flow, e.g., by depressurizing the nipple.

The positive pressure provided by the control valve 10 may be between 0 mmHg to 400 mmHg, e.g., about 25 mmHg. In some embodiments, the positive pressure is 0 mmHg, e.g., open atmospheric pressure. In some embodiments, the pressure source 9 and control valve 10 are contained within the same feature.

In some embodiments, the modular breast pump system 1 may include an open-close valve. In some embodiments, the control valve includes an open-close valve. The open-close valve may be configured to open and close fluidic communication between a first element and a second element. In some embodiments, the first element can include a reservoir 3, fluid conduit 5, a pressure chamber 20, a milk chamber 21, a manifold, or the ambient atmosphere. In some embodiments, the second element can include a reservoir 3, fluid conduit 5, a pressure chamber 20, a milk chamber 21, a manifold, or the ambient atmosphere.

The modular breast pump system 1 may include one or more one directional valves 15 or anti-backflow valves, e.g., a plurality of one directional valves 15.

The one directional valve 15 may be a duckbill valve or other type of one directional valve.

In some embodiment, the one directional valve 15 may be placed in a fluid conduit 5, e.g., to prevent backflow of milk. In some embodiments, the one directional valve 15 is configured to allow fluid flow from the milk chamber 21 to the reservoir 3. In some embodiments, the one directional valve 15 is disposed in the first fluid conduit 5. In some embodiments, the one directional valve 15 is disposed at the outlet 22 of the milk chamber 21, e.g., FIG. 13D. In some embodiments, the one directional valve 15 is disposed in the diaphragm 18. In some embodiments, the one directional valve 15 may prevent air from entering the first fluid conduit 5, thereby maintaining negative pressure to move the milk.

In some embodiments, the one directional valve 15 is configured to allow fluid flow from the control valve 10 to the breast cup 2 or pressure source 9. In other embodiments, the one directional valve 15 may allow air to flow to the first fluid conduit from the control valve 10, such that the negative pressure from the pressure source 9 fluctuates to express milk from the breast. Thus, in some embodiments, the one directional valve 15 follows the control valve 10 in a fluid conduit 5, e.g., in the third fluid conduit 5. In some embodiments, the one directional valve 15 is between the breast cup 2 and the reservoir 3, such that milk cannot backflow from the reservoir 3. FIG. 2 shows a reservoir 3 in an enclosure 4 having a one directional valve 15.

In some embodiments, the inlet to the milk chamber 21 includes a one directional valve, e.g., as shown in FIG. 5. In some embodiments, the one directional valve is a duckbill valve. The modular breast pump system 1 may further include at least one relief valve. In some embodiments, at least one fluid conduit 5 includes a relief valve. In some embodiments, the breast cup 2 includes a relief valve. If vacuum at the breast becomes too strong the relief valve may provide a release of negative pressure to prevent discomfort for the user. A relief valve may be placed such that if a predetermined maximum vacuum level is exceeded in a fluid conduit 5, such as greater than 250 mmHg vacuum (−250 mmHg pressure), the relief valve is able to provide positive pressure. The relief valve can be in the form of a spring and ball, pin and O-ring, or other equivalent mechanical means of providing pressure relief. In some embodiments, the user may activate the release valve. In some embodiments, a user may twist a relief valve to lower or shut off suction. In some embodiments, the modular breast pump system 1 includes one or more quick-release valves. In some embodiments, the outlet of breast cup 2, e.g., the outlet 22 of the milk chamber 21, includes a quick-release valve such that a fluid conduit 5 may be easily connected to the milk chamber 21.

In some embodiments, the modular breast pump system 1 is configured to permit a slow leak of pressure. The slow leak of pressure may be to the ambient atmosphere. A slow leak aids in slowly venting pressure during breast pumping. In some embodiments, the breast cup 2 includes a slow leak of pressure. In some embodiments, the breast shield 19 includes a slow leak of pressure. In some embodiments, the breast shield 19 includes an imperfect seal configured to provide a slow leak. In some embodiments, the breast shield 19 includes an orifice to provide a slow leak. In some embodiments, the slow leak includes a leak rate that would not decrease the maximum negative pressure of a pressure cycle by more than 10% (e.g., 9%, 8%, 7%, 6%, 5%, 4%, 3%, 2%, or 1%). In some embodiments, the slow leak provides a positive pressure to the breast cup 2 that is equivalent to the maximum applied negative pressure over a period of time from about 0.5 minutes to about 60 minutes (e.g., about 0.5 minutes to about 1 minute, about 0.5 minutes to about 5 minutes, about 0.5 minutes to about 10 minutes, about 0.5 minutes to about 15 minutes, about 0.5 minutes to about 30 minutes, about 1 minute to about 5 minutes, about 2 minutes to about 8 minutes, about 3 minutes to about 7 minutes, about 5 minutes to about 10 minutes, about 5 minutes to about 15 minutes, about 5 minutes to about 30 minutes, about 10 minutes to about 15 minutes, about 10 minutes to about 20 minutes, about 15 minutes to about 30 minutes, about 15 minutes to about 45 minutes, about 30 minutes to about 45 minutes, about 30 minutes to about 60 minutes or about 45 minutes to about 60 minutes). A slow leak valve may be configured to provide the slow leak of pressure. For example, if a maximum pressure of 100 mmHg is applied to the breast cup 2, e.g., the nipple tunnel of the breast shield 19, the slow leak may provide 10 mmHg of positive pressure every minute for 10 minutes.

In some embodiments, the modular breast pump system 1 includes a slow leak valve. The slow leak valve may be configured to provide a slow leak of pressure to the ambient atmosphere. In some embodiments, the slow leak valve includes a leak rate that would not decrease the maximum negative pressure of a pressure cycle by more than 10% (e.g., 9%, 8%, 7%, 6%, 5%, 4%, 3%, 2%, or 1%) when negative pressure is being applied to the breast cup 2.

### Sensors

The modular breast pump system 1 may also include at least one sensor. At least one sensor may detect a baseline value, e.g., an initial pressure in the breast cup 2. Throughout breast pumping the sensor may continue detecting. Data are compared throughout breast pumping to the initially detected baseline. Sensors include weight sensors, load cells, temperature sensors, pressure sensors, pH sensors, flow sensors, viscosity sensors, volume sensors, etc. Sensors may be in the base module 7, the portable module 6, the breast cup 2, at least one fluid conduit 5, in the pump system, in the reservoir 3, in the reservoir enclosure 4, or on an external surface of the modular breast pump system.

In some embodiments, the modular breast pump system 1 includes at least one weight sensor or load cell. In some embodiments, the weight sensor is disposed in the bottom of the reservoir 3. In some embodiments, the weight sensor is disposed in the bottom of the reservoir enclosure 4, e.g., in the base and/or portable module 6, underneath one or a plurality of reservoirs 3, such that the weight of the reservoirs may be measured. In some embodiments, the weight sensor is disposed with a weighing plate 34. Weight sensors may track the weight of the reservoirs 3 over time. In some embodiments, weight data may be used to determine flow rate of milk during a pumping session. In some embodiments, weight sensors may be used to determine the volume of milk inside of a reservoir 3.

In some embodiments, the modular breast pump system 1 includes at least one temperature sensor, e.g., a thermocouple. In some embodiments, the breast pump includes a plurality of temperature sensors. Temperature sensors may provide time history data. Using the circadian baseline, deviations from baseline may be identified. In some embodiments, the reservoir 3 includes a temperature sensor. A change in temperature of milk may indicate spoiled milk.

The modular breast pump system 1 may include at least one pressure sensor. In some embodiments, the breast pump includes a plurality of pressure sensors. Pressure sensors may include diaphragms, strain gauges, piezoresistive pressure sensors, capacitive pressure sensors, or electromagnetic sensors. Pressure sensors may be used to measure the pressure in the breast cup 2, e.g., in the pressure chamber 20, milk chamber 21, and/or breast shield 19. Data from the pressure sensor may be used to determine the pressure used to express milk from the nipple. Pressure sensors are also advantageous in that they may detect high pressure levels which may cause discomfort for the user. In some embodiments, the breast cup 2 includes a pressure sensor. In some embodiments, the reservoir 3 includes a pressure sensor. In some embodiments, each reservoir 3 includes a pressure sensor. A pressure sensor in a reservoir can be advantageous in determining over pressurization of the reservoir 3, e.g., as a result of milk being pumped into the reservoir 3.

The modular breast pump system 1 may include at least one pH sensor. In some embodiments, the breast pump includes a plurality of pH sensors. In some embodiments, the pH sensor is an ion sensitive glass electrode.

The modular breast pump system 1 may include at least one flow sensor. In some embodiments, the breast pump includes a plurality of flow sensors. In some embodiments, a fluid conduit 5 includes a flow sensor. A flow sensor may detect the rate of fluid flow in the fluid conduit 5.

The modular breast pump system 1 may include at least one volume sensor. In some embodiments, the breast pump includes a plurality of volume sensors. In some embodiments, the reservoir 3 includes a volume sensor. A volume sensor may detect the level of milk in the reservoir 3.

The modular breast pump system 1 may include at least one optical sensor. In some embodiments, the breast pump includes a plurality of optical sensors. The optical sensor may include a light source, e.g., an LED light source, and associated photodetector. The light source in one example emits light at different wavelengths within the visible spectrum, including a violet light, a blue light, a green light, a yellow light, an orange light and a red light. In various embodiments, the optical sensor includes a singular light source or a plurality of light sources, similarly the photodetector may include a single photodetector or a plurality of photodetectors. In some embodiments, the reservoir 3 may include an optical sensor.

In some embodiments, the optical sensor may sense movement of the breast cup 2 on the breast. Sensing of movement of the breast cup 2 on the breast is advantageous in that it may measure how much the breast cup 2 moves during use, e.g., such as through the night or while moving. In some embodiments, the modular breast pump system 1 can alert the user if it becomes unaligned or loose. In some embodiments, the optical sensor is in view of a portion of the skin of the user. In some embodiments, the breast cup 2, e.g., an outer surface of breast cup 2, e.g., the housing, includes the optical sensor. In some embodiments, the optical sensor is mounted on the housing.

In some embodiments, the optical sensor may sense the clarity of the milk in the reservoir 3. The clarity of the milk may be indicative of fat content, e.g., a lower clarity may be indicative of a higher fat content.

The modular breast pump system 1 may include at least one viscometer. In some embodiments, the modular breast pump system 1 includes a plurality of viscometers. In some embodiments, the reservoir 3 includes a viscometer. The viscometer may sense the viscosity of the milk, e.g., the milk in the reservoir 3.

In some embodiments, the modular breast pump system 1 and/or the milk distribution system 28 includes a Hall effect sensor. A Hall effect sensor is a sensor which detects the presence and magnitude of a magnetic field using the Hall effect. A Hall effect sensor is advantageous in that it may allow the modular breast pump system 1 and/or the milk distribution system 28 to collect information on whether the system completes actions. The information collected by the Hall effect sensor may be used to provide assurance and aid in diagnosing any errors.

### Additional Elements

In some embodiments, the modular breast pump system 1 includes a heating system. A heating system is advantageous in that the breast cup 2 may be heated prior to or during breast pumping for improved comfort. In some embodiments, the heating system is disposed in the breast shield 19. In some embodiments, the heating system includes insulation, an insertion heater, a resistive heater, a thermoelectric heater, a heating film, a heating pad, a heating wire, or a pneumatic heating tube.

In some embodiments, the modular breast pump system 1 includes a pressure outlet. The pressure outlet may or may not include a valve 24. In some embodiments, each pressure outlet includes a valve 24. In some embodiments, the pressure outlet is a breather tube. In some embodiments, the pressure outlet is an orifice, e.g., the reservoir 3 or the lid of the reservoir 3 may include an orifice. In some embodiments, the milk distribution system 28 includes a pressure outlet in fluidic communication with each reservoir 3.

In some embodiments, the reservoir 3 includes a pressure indicator, e.g., a flexible membrane. A pressure indicator, e.g., a flexible membrane, may be advantageous in indicating if there is negative or positive pressure within the reservoir 3. Negative pressure may be the result of hot milk being cooled when the reservoir 3 is closed to the ambient atmosphere and/or the milk distribution system 28.

In some embodiments, the modular breast pump system 1 includes a waste reservoir. In some embodiments, following a breast pumping session, the modular breast pump system 1 may flush the system with a cleaning fluid, wherein the cleaning fluid is transported to a waste reservoir. In some embodiments, the cleaning fluid is water. In some embodiments, the cleaning fluid is air. In some embodiments, the modular breast pump system 1 is configured to be flushed with a pressure source, e.g., positive pressure source or a negative pressure source, e.g., a vacuum pump.

In some embodiments, the modular breast pump system 1 may include an anti-bacterial coating, e.g., in the reservoir 3 or the fluid conduits 5.

The modular breast pump system 1 may include a display. The display may be disposed on the portable module 6 and/or the base module 7. The display may be a screen. In some embodiments, the screen is a touchscreen.

The modular breast pump system 1 may include a button and/or an icon. The button and/or icon may be disposed on the portable module 6 and/or the base module 7. The button and/or icon may include a light. In some embodiments, the light includes one or more light emitting diodes (LEDs).

The modular breast pump system 1 may include a timer. The drive unit 11 and/or control unit may include the timer. The timer may be disposed in the portable module 6 and/or the base module 7.

The modular breast pump system 1 may include an alarm. The alarm may be disposed in the portable module 6 and/or the base module 7. The alarm may be a light, a vibration element, or a sound element. The alarm may be configured to sound in the event of an error (e.g., the pumping system is improperly assembled), the ending of a pumping session, a temperature increase in the milk in the reservoir 3, a full reservoir 3, and/or the start of an upcoming preprogrammed pumping session.

The modular breast pump system 1 may include a power switch 14 or button. The power switch 14 or button may enable a user to power on or off the modular breast pump system 1. The power switch 14 or button may be disposed on the outside of the portable module 6 and/or the base module 7. Alternatively, or in addition, the power switch 14 or button may be disposed on the breast cup 2. Alternatively, or in addition, the power switch 14 or button may be disposed on the reservoir enclosure 4. However, in some embodiments, the user may power on or off the modular breast pump system 1 remotely, such as through an application on an external computer, e.g., a mobile device.

### Wearability

The modular breast pump system 1 may include a handle, belt, strap, fastener, or garment to secure the portable module 6 to the user. A strap may be attached to the portable module 6, such that a user may carry the portable module 6 with the strap, e.g., over the shoulder or cross-body. A fastener, e.g., a clip, a hook, a zipper, a magnet, a pin, a button, a snap, a hook and loop fastener (e.g., VELCRO®), or a combination thereof, may be attached to the portable module 6 to be attached releasably, e.g., clipped to the clothing, to the user. In some embodiments, the portable module 6 may include a garment, Wherein the portable module 6 is releasably attached to the garment, e.g., a belt.

In some embodiments, the portable module 6 may be worn such that all the steps of the method may be carried out hands-free, and/or with all elements of the modular breast pump system, with the exception of the base module 7, releasably attached to the user's person.

The modular breast pump system 1 may include a garment to secure the breast cup 2 to the user. The garment may secure the breast cup 2 in place, and/or stabilize breast cup 2 for milk expression. In some embodiments, the breast cup 2 is inserted into the garment. In some embodiments, the portable module 6 and/or the breast cup 2 are secured to the same garment.

The garment may be a bra, a bustier, a sports bra, a shirt, a tank top, a bandeau, a strap, a dress, a nightgown, or a nursing bra that provides support to a wearer and/or at least a portion of a modular breast pump system 1. In some embodiments, the garment includes polyester, cotton, linen, satin, organdy, rayon, taffeta, broad cloth, poplin, velour, gauze, canvas, shirting, muslin, tweed, georgette, crepe, wool, twill, gabardine, denim, or drill. In some embodiments, the garment is machine washable.

In some embodiments, the breast cup 2 and/or the portable module 6 is held in place by compression provided by the garment. The garment may provide a level of compression of the breast cup 2 to the breast such that the subject may move, rest, and/or sleep with the breast cup continuing to be secured to the breast. Securement of the breast cup 2 to the breast during movement improves safety and reduces milk leakage. The garment may include a flexible or stretch material, e.g., nylon, elastic, or spandex. The garment may include portions of varying compression, e.g., high compression portions and low compression portions. For example, the material of the garment contacting the breast cup may include a high compression portion. The garment may apply a compression of at least 5 mmHg, e.g., at least 10 mmHg, 15 mmHg, 20 mmHg, 25 mmHg, 30 mmHg, 35 mmHg, 40 mmHg, 45 mmHg, 50 mmHg, 55 mmHg, 60 mmHg, 65 mmHg, 70 mmHg, 75 mmHg, 80 mmHg, 85 mmHg, 90 mmHg, 95 mmHg, 100 mmHg, 125 mmHg, or 150 mmHg. In other embodiments, the garment may apply a compression from about 5 mmHg to about 100 mmHg, e.g., about 5 mmHg to about 10 mmHg, about 5 mmHg to about 15 mmHg, about 10 mmHg, to about 20 mmHg, about 5 mmHg to about 25 mmHg, about 5 mmHg to about 30 mmHg, about 5 mmHg to about 35 mmHg, about 5 mmHg to about 40 mmHg, about 5 mmHg to about 45 mmHg, about 5 mmHg to about 50 mmHg, about 5 mmHg to about 60 mmHg, about 5 mmHg to about 70 mmHg, about 5 mmHg to about 80 mmHg, about 5 mmHg to about 90 mmHg, about 5 mmHg to about 100 mmHg, about 10 mmHg to about 20 mmHg, about 10 mmHg to about 30 mmHg, about 10 mmHg to about 40 mmHg, about 10 mmHg to about 50 mmHg, about 10 mmHg to about 75 mmHg, about 10 mmHg to about 100 mmHg, about 20 mmHg to about 30 mmHg, about 20 mmHg to about 40 mmHg, about 20 mmHg to about 50 mmHg, about 25 mmHg to about 50 mmHg, about 25 mmHg to about 75 mmHg, about 25 mmHg to about 100 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 50 mmHg to about 60 mmHg, about 50 mmHg to about 60 mmHg, about 50 mmHg to about 70 mmHg, about 50 mmHg to about 80 mmHg, about 50 mmHg to about 100 mmHg, about 60 mmHg to about 70 mmHg, about 60 mmHg to about 80 mmHg, about 60 mmHg to about 70 mmHg, about 60 mmHg to about 80 mmHg, about 60 mmHg to about 90 mmHg, about 60 mmHg to about 100 mmHg, about 70 mmHg to about 80 mmHg, about 70 mmHg to about 90 mmHg, about 70 mmHg to about 100 mmHg, about 80 mmHg to about 90 mmHg, about 80 mmHg to about 100 mmHg, or about 90 mmHg to about 100 mmHg.

The garment provides access to at least one breast to facilitate breastfeeding and/or pumping. Several embodiments are described to provide access to at least one breast. In some embodiments, the user may pull the garment away from their skin, and insert the breast cup 2 underneath the garment from below or above the garment. In some embodiments, the garment may have openings formed or defined in the material that makes up the garment to provide an opening for access to at least one of the wearer's breasts. In some embodiments, the opening in the garment has a smaller diameter than the largest diameter of the breast cup 2. In some embodiments, the breast cup 2 and/or housing are inserted into at least one opening formed in the material of the garment, and compression of the material secures it to the breast. This configuration is advantageous such that the breast cup 2 may not be pulled out from the garment unintentionally. For example, the nipple tunnel 13 of the breast cup 2 may be inserted through the opening in the garment such that the wide portion 8 of the breast cup 2 may contact the garment, but not pass through. The nipple tunnel 13 of the breast cup may then be releasably connected to a fluid conduit 5.

The opening in the garment may have a diameter from about 5 mm to about 250 mm, e.g., from about 5 mm to about 10 mm, about 5 mm to about 20 mm, about 5 mm to about 25 mm, about 5 mm to about 30 mm, about 5 mm to about 40 mm, about 5 mm to about 50 mm, about 5 mm to about 75 mm, about 5 mm to about 100 mm, about 10 mm to about 20 mm, about 10 mm to about 25 mm, about 10 mm to about 30 mm, about 10 mm to about 40 mm, about 10 mm to about 50 mm, about 20 mm to about 25 mm, about 20 mm to about 30 mm, about 20 mm to about 40 mm, about 20 mm to about 50 mm, about 25 mm to about 50 mm, about 25 mm to about 75 mm, about 30 mm to about 40 mm, about 30 mm to about 50 mm, about 40 mm to about 50 mm, about 50 mm to about 75 mm, about 50 mm to about 100 mm, about 50 mm to about 125 mm, about 50 mm to about 150 mm, about 50 mm to about 75 mm, about 75 mm to about 100 mm, about 75 mm to about 125 mm, about 75 mm to about 150 mm, about 75 mm to about 175 mm, about 75 mm to about 200 mm, about 100 mm to about 125 mm, about 100 mm to about 150 mm, about 100 mm to about 175 mm, about 100 mm to about 200 mm, about 125 mm to about 150 mm, about 125 mm to about 175 mm, about 125 mm to about 200 mm, about 150 mm to about 175 mm, about 150 mm to about 200 mm, about 150 mm to about 250 mm, or about 200 mm to about 250 mm. The opening may have a diameter less than 100 mm, e.g., less than 90 mm, 80 mm, 70 mm, 60 mm, 50 mm, 40 mm, 30 mm, 25 mm, 20 mm, 10 mm, or 5 mm.

The garment may include at least one layer, e.g., two layers. The two layers may overlap such that they may be pulled apart to reveal at least one breast. In some embodiments, the garment includes at least two layers, and the breast cup 2 and/or housing are secured to the breast by positioning between at least two layers. Alternatively, or additionally, the garment may include snaps, buttons, or a zipper which may be opened to provide access to at least one breast. In addition to providing access to at least one breast, the modular breast pump system 1 may include a fastener for securing the breast cup 2 and/or housing to the garment. The modular breast pump system 1 may also include a fastener for securing a fluid conduit 5. The fastener may be used to route the fluid conduit 5 to an advantageous location (e.g., under the arm, or under the garment), such that the fluid conduit does not impede the user's freedom of motion. The modular breast pump system 1 may also include a fastener system for the portable module 6, and/or the reservoir 3, and/or reservoir enclosure 4. The fastener may attach the portable module 6, and/or the reservoir 3, and/or reservoir enclosure 4 to the user, such that they are able to conduct their daily activities while breast pumping. The fastener may include a clip, a strap, a hook, a zipper, a magnet, a pin, a button, a snap, a hook and loop fastener (e.g., VELCRO®), or a combination thereof. The fastener may fasten the portable module 6, and/or the reservoir 3, and/or reservoir enclosure 4 to the user in such a way that they may have freedom of motion, e.g., fasten the reservoir 3 and/or reservoir enclosure 4 to the waist of the user.

Securement to the breast may be further improved by negative pressure provided by the pressure source 9.

The modular breast pump system 1 may be reversibly connected to the garment before or after the garment is donned. In some embodiments, the portable module 6, the breast cup 2, and/or housing are reversibly secured to the garment and then the garment is donned. In some embodiments, the garment is donned, and then the portable module 6, the breast cup 2, and/or housing are reversibly secured to the garment. In some embodiments, the portable module 6, the breast cup 2, and/or housing are irreversibly secured to the garment. In some embodiments, the fluid conduit 5 is reversibly secured to the breast cup 2 and/or the portable module 6 after the garment is donned. In some embodiments, the fluid conduit 5 is reversibly secured to the breast cup 2 and/or the portable module 6 prior to the garment being donned.

In some embodiments, the outer surface of breast cup 2 has a lower coefficient of friction than the inner surface of the garment to allow freedom of movement. Without wishing to be bound to theory, if there is a lower coefficient of friction between the breast cup 2 and the garment than between the breast cup 2 and the breast, then relative movement of the garment is less likely to move the breast cup 2 out of position. In some embodiments, the coefficient of friction between the outer surface of the breast cup 2, e.g., the housing, and the garment is less than 0.4 (e.g., 0.35, 0.3, 0.25, 0.2, 0.15, 0.1, or 0.05). In some embodiments, the outer surface of the breast cup 2, e.g., the housing, can include a smooth material. In some embodiments, the outer surface of the breast cup 2, e.g., the housing, can include a smooth plastic or fabric. In some embodiments, the outer surface of the breast cup 2, e.g., the shield 19, can include PET, PP, PTFE, polyethylene, HDPE, LDPE, PC, nylon, acetal, PEEK, PPS, or polyester. In some embodiments, the outer surface of the breast cup 2, e.g., the housing, can include cotton, linen, satin, organdy, rayon, taffeta, broad cloth, poplin, velour, gauze, canvas, shirting, muslin, tweed, georgette, crepe, wool, twill, gabardine, denim or drill.

### Modular Breast Pump System Control

The modular breast pump system 1 may include controls that are operable by a user to select or modify at least one of: pumping program or mode, frequency of pumping cycle, maximum suction pressure achieved during a pumping cycle, latch suction pressure achieved during the pumping cycle, volume of the pressure chamber 20, pumping force, pumping session time, and distribution of milk to reservoirs 3. The controls may be present on the surface of the base and/or portable module 6, e.g., on the display. Alternatively, or in addition, the controls may be present in an application on an external computer, e.g., a mobile device. Alternatively, the modular breast pump system 1 is solely controlled through an application on an external computer, e.g., a mobile device. Control of the modular breast pump system 1 through an application on an external computer is advantageous in that a user may control the device in any position.

In some embodiments, the modular breast pump system 1 can have a wait time set, e.g., through the display or an application on an external computer, such that the user has time to contact the breast cup 2 to their breast before the modular breast pump system 1 begins pumping.

Control of the modular breast pump system 1 may be customized. The customized pump functions include modifications to at least one of: maximum suction pressure level, latch suction pressure level, suction pressure waveform over a pumping cycle, phases of extraction or feeding times, rest times, heating temperatures and times, vibration frequency and duration, pumping session time, and distribution of milk to reservoirs 3. The user may input at least one pump program into the modular breast pump system 1, e.g., through the display or application on an external computer. For example, the user may set the number of minutes that the modular breast pump system 1 will run before shutting off automatically and/or the user may set a time for the modular breast pump system 1 to engage during the night. Custom pump programming is advantageous in that user may control how long they pump for so that they may go about their daily activities, resting, or sleeping, without the worry that the pump will continue endlessly. In some embodiments, the pumping frequency may be programmed to slow down gradually during operation, e.g., towards the end of a preprogrammed pumping time.

### Wireless

The control unit may be configured to send signals to and receive signals from an external computer, e.g., a mobile device.

The control unit may include a transmission element, e.g., a wireless transmission element, and receiver element for wirelessly sending signal to and receiving signals from the external computer. In some embodiments, the transmission element is a BLUETOOTH® transmission element. In some embodiments, the transmission element is a BLUETOOTH® low energy (BLE) transmission element.

The external computer may include a processor and instructions, which when executed, cause the processor to customize pump functions and send customized pump functions to the control unit. In some embodiments, the custom pump functions are based on the signals received from the control unit, such as measurements taken by sensors. In some embodiments, the user is able to program different custom pump programs, e.g., a daytime program and a nighttime program.

The external computer may include a processor and Instructions, which when executed, cause the processor to calculate volume of milk extracted and track expression efficiency and monitor it over time. In some embodiments, the calculation of milk extracted, and expression efficiency are based on signals received from the control unit, such as measurements taken by the sensors.

The external computer may include a processor and instructions, which when executed, cause the processor to transport milk from a breast pumping session to a reservoir, including transporting milk from a first breast pumping session to a first reservoir, and milk from a second breast pumping session to a second reservoir, e.g., in the base module.

The external computer may include a processor and instructions, which when executed, cause the processor to track inventory of previous pumping sessions, including tracking at least one of dates of the previous pumping sessions, volumes pumped in the previous pumping sessions, and specific tracking numbers for specific milk collection containers into which milk has been pumped in the previous pumping sessions.

The external computer may include a processor and instructions, which when executed, cause the processor to one or more of monitor remaining battery power of the battery and output a warning when the battery reaches a predetermined low level of charge.

The external computer may include a processor and instructions, which when executed, cause the processor to display in the application screen or on display of the modular breast pump system 1 the time left in the pumping session, measurements taken by the at least one sensor, and/or one or more photos of the user's choosing on the display.

### Breast Pumping and Related Conditions

Many mothers rely on breast pumping to provide milk to their infants, stimulate lactation, or relieve engorgement. For the mothers of newborns, breastfeeding or breast pumping often occurs every two hours, interrupting daytime activities, work, errands, social activities, and sleep schedules. Consistent breastfeeding or breast pumping, however, may be difficult to achieve discreetly in public settings and can require women to take time away from daily activities and interactions, as well as contribute to a number of conditions in women, in particular due to a lack of rest. Improved modular breast pump systems may lessen or reduce the time constraints on women and occurrences of these conditions, while still allowing women to breast pump consistently.

Current commercially available breast pumps are not equipped to allow women to perform all steps of a breast pumping session in a public place, such as a park, stadium, or a restaurant. Firstly, a majority of pumps are electric pumps, which require access to a working power outlet to function. A working outlet may not be accessible in many locations such as in a park or on an airplane. Second, portable pumps that are powered either by batteries or manual expression typically have large profiles, where the breast cup is attached directly to the reservoir or milk bottle such that they protrude directly perpendicular to the user's chest. These pumps cannot be used with any discretion, as clothing, e.g., a shirt or blouse, must be removed to utilize the pumps. Additionally, these pumps often lack the power required to fully express all the milk in a user's breast. Thirdly, no portable pump exists with off-body milk collection. A pump with on-body milk collection requires the milk to stay in the pumping unit until the whole unit is removed. For an in-bra pump, the user would have to remove the entire pump to access the milk, and leaving the pump in place after a pumping session may lead to spoiled milk and leaks.

The systems and methods described herein are advantageous because they include a portable module that allows breast pumping on the go and also a base module that allows breast pumping at home. Thus, the user can breast pump in all environments with a single modular system.

### Methods of Use

The present disclosure provides improved methods of breast pumping.

The present disclosure provides a method of breast pumping by: (i) providing a modular breast pump system 1 of the disclosure including: (ii) contacting a human breast with the breast cup; and (iii) applying an alternating first pressure and second pressure such that milk is moved from the human breast to the reservoir 3, in which the first pressure and the second pressure are functions of a negative pressure and a positive pressure. The breast pumping system is optionally mobile before, during, and/or after pumping.

In some embodiments, the pumping occurs using the portable module when decoupled from the base module. In other embodiments, the pumping occurs using the base module when mated to the portable module. In some embodiments, the method further includes picking up, wearing, and/or holding the portable module before, during, and/or after the pumping session. In some embodiments, the method further includes releasably attaching the portable module to a subject prior to, during, and/or after completion of a pumping session, e.g., via a strap, belt, or fastener. In some embodiments, the method further includes the subject moving, reclining, resting, sleeping, or traveling. In some embodiments, the method further includes the subject being located outdoors. In some embodiments, the method includes the subject moving between a first location and a second location. In some embodiments, the method further includes mating the portable module with the base module.

In some embodiments, the method includes recharging the power source in the portable module. In some embodiments, the method includes connecting the portable module to the base module to charge the power source in the portable module. In some embodiments, the method includes replacing batteries in the portable module.

In some embodiments, the method includes a first pumping session, wherein the portable module and the base module are disconnected. In some embodiments, the method includes a first pumping session and a second pumping session, wherein the portable module and the base module are disconnected. In some embodiments, the method includes a first pumping session, wherein the portable module and the base module are disconnected; connecting the portable module and the base module; disconnecting the portable module and the base module; and performing a second pumping session with the portable module. In some embodiments, the method includes a first pumping session, wherein the portable module and the base module are disconnected; connecting the portable module and the base module; and performing a second pumping session. In some embodiments, the method includes performing a first pumping session, wherein the base and portable modules are mated. In some embodiments, the method includes a first pumping session and a second pumping session, wherein the base and portable modules are mated. In some embodiments, the method includes a first pumping session, wherein the base and portable modules are mated; disconnecting the portable module and the base module; and performing a second pumping session with the portable module. Advantageously, breast cups need not be removed from the user when decoupling the portable module from the base module. Fluid conduits may need to be moved from the reservoir enclosure in the base module to the reservoir enclosure in the portable module or replaced.

In some embodiments, the method includes a first pumping session and a second pumping session, wherein the breast cups are not removed between the first pumping session and the second pumping session. In some embodiments, the breast cups are removed between the first pumping session and the second pumping session.

In some embodiments, the method further includes releasably securing a reservoir in the portable module.

In some embodiments, the method includes releasably securing a reservoir in the portable module; providing a fluidic connection between the breast cup and the reservoir; and pumping the human breast such that milk is expressed into the breast cup and travels through the fluid conduit to the reservoir. In some embodiments, the method further includes removing the reservoir from the portable module after the conclusion of the pumping session, and placing the reservoir in a fridge, freezer, auxiliary reservoir enclosure, disposing of the milk in the reservoir, or feeding the milk in the reservoir to a child.

In some embodiments, the method includes securing the breast cup to a human breast; releasably securing a first reservoir in the portable module; providing a fluidic connection between the breast cup and the first reservoir; performing a first pumping session, such that expressed milk travels from the breast cup into the first reservoir; removing the first reservoir from the portable module; releasably securing a second reservoir in the portable module; and pumping in a second pumping session, such that expressed milk travels from the breast cup into the second reservoir. In some embodiments, the first reservoir is releasably secured in the portable module prior to securing the breast cup on the human breast.

In some embodiments, the time between one breast pumping session and another breast pumping session (e.g., between a first breast pumping session and a second breast pumping session, a second breast pumping session and a third breast pumping session, a third breast pumping session and a fourth breast pumping session, etc.) is from about 30 minutes to about 240 minutes (e.g., about 30 minutes to about 45 minutes, about 30 minutes to about 60 minutes, about 30 minutes to about 90 minutes, about 30 minutes to about 120 minutes, about 30 minutes to about 180 minutes, about 45 minutes to about 60 minutes, about 45 minutes to about 90 minutes, about 45 minutes to about 120 minutes, about 60 minutes to about 90 minutes, about 60 minutes to about 120 minutes, about 50 minutes to about 180 minutes, about 60 minutes to about 210 minutes, about 90 minutes to about 120 minutes, about 90 minutes to about 180 minutes, about 90 minutes to about 210 minutes, about 90 minutes to about 240 minutes, about 120 minutes to about 180 minutes, about 120 minutes to about 210 minutes, about 120 minutes to about 240 minutes, about 180 minutes to about 210 minutes, about 180 minutes to about 240 minutes, about 210 minutes to about 240 minutes, about 30 minutes, about 45 minutes, about 60 minutes, about 85 minutes, about 90 minutes, about 105 minutes, about 120 minutes, about 135 minutes, about 150 minutes, about 165 minutes, about 180 minutes, about 195 minutes, about 210 minutes, about 225 minutes, or about 240 minutes). In some embodiments, the modular breast pump system 1 may enter a waiting phase between breast pumping sessions where the breast cup 2 and/or manifold 29 is not in fluidic communication with any reservoir 3. In some embodiments, following a breast pumping session, the modular breast pump system 1 places the breast cup 2 and/or manifold 29 in fluidic communication with a second reservoir 3. The second reservoir 3 may be empty.

In some embodiments, milk from the first breast pumping session is transported to a first reservoir, and milk from the second breast pumping session is transported to a second reservoir. Likewise, milk from a third, fourth, fifth, sixth, seventh, eighth, ninth, or tenth breast pumping session may be transported into a third, fourth, fifth, sixth, seventh, eighth, ninth, or tenth reservoir, respectively. In some embodiments, the method includes removing fluidic communication between the breast cup 2 and all of the reservoirs 3 between breast pumping sessions. In some embodiments, a plurality of valves is closed to remove fluidic communication between the breast cup 2 and all of the reservoirs 3. Once full, a reservoir may be removed and replaced with an empty reservoir.

The method may further include assembling the modular breast pump system 1. In some embodiments, the method includes releasably connecting at least one fluid conduit 5 to the base and/or portable module. In some embodiments, the method further includes releasably connecting at least one fluid conduit to the breast cup 2. In some embodiments, the method includes releasably connecting at least one fluid conduit to the reservoir 3. In some embodiments, the method includes connecting the first fluid conduit 5 to the breast cup 2 and the reservoir 3. In some embodiments, the method includes connecting the second fluid conduit 5 to the pressure source 9 and the breast cup 2.

Releasable connections between the modular breast pump system 1 are advantageous for ease of cleaning.

The modular breast pump system 1 is advantageous in that the user may be able to breast pump a plurality of times following a one-time assembly. Prior to the first use, the user may secure the breast cup to a breast and/or garment to their body. Then, in some embodiments, the user does not need to remove the breast cup 2 and/or garment between a plurality of breast pumping sessions, e.g., two, three, four, five, six, seven, eight, nine, or ten breast pumping sessions. The modular breast pump system 1 is further advantageous in that the user may be able to breast pump in a reclined position. As the modular breast pump system 1 may be used for an extended time, e.g., overnight, the user's ability to place the wearable component on one time, yet still use the pump several times without removal of the garment will allow for uninterrupted sleep during at least one breast pumping session. A pre-programmed pumping schedule may initiate the modular breast pump system 1 at least once, e.g., two, three, four, five, six, seven, eight, nine, ten times, etc. In some embodiments, the milk distribution system 28 can distinguish between pumping sessions based on the pre-programmed breast pumping schedule. For example, on the initiation of a new breast pumping session, the breast pump system 1 and/or the milk distribution system 28 can divert the milk flow via a manifold 29 to an empty reservoir 3.

In some embodiments, the method includes pumping milk from the human breast to induce milk from a first breast pumping session to flow from a nipple to the manifold in the base module via the breast cup 2; providing fluidic communication between the inlet 30 of the manifold 29 and a first reservoir 3 to allow milk from the first breast pumping session to flow into the first reservoir 3; removing fluidic communication between the inlet 30 of the manifold 29 and the first fluid reservoir 3; pumping the human breast to induce milk from a second breast pumping session to flow from the nipple to the manifold 29 via the breast cup 2; and providing fluidic communication between the inlet 30 of the manifold 29 and a second reservoir 3 to allow milk from the second breast pumping session to flow into the second reservoir 3. In some embodiments, the time between first and second pumping sessions is from about 30 minutes to about 240 minutes (e.g., about 30 minutes to about 45 minutes, about 30 minutes to about 60 minutes, about 30 minutes to about 90 minutes, about 30 minutes to about 120 minutes, about 30 minutes to about 180 minutes, about 45 minutes to about 60 minutes, about 45 minutes to about 90 minutes, about 45 minutes to about 120 minutes, about 60 minutes to about 90 minutes, about 60 minutes to about 120 minutes, about 50 minutes to about 180 minutes, about 60 minutes to about 210 minutes, about 90 minutes to about 120 minutes, about 90 minutes to about 180 minutes, about 90 minutes to about 210 minutes, about 90 minutes to about 240 minutes, about 120 minutes to about 180 minutes, about 120 minutes to about 210 minutes, about 120 minutes to about 240 minutes, about 180 minutes to about 210 minutes, about 180 minutes to about 240 minutes, about 210 minutes to about 240 minutes, about 30 minutes, about 45 minutes, about 60 minutes, about 85 minutes, about 90 minutes, about 105 minutes, about 120 minutes, about 135 minutes, about 150 minutes, about 165 minutes, about 180 minutes, about 195 minutes, about 210 minutes, about 225 minutes, or about 240 minutes).

In some embodiments, the method further includes removing fluidic communication between the inlet 30 of the manifold 29 and the second fluid reservoir 3; and providing fluidic communication between the inlet 30 of the manifold 29 and a third reservoir 3; and pumping the human breast to induce milk from a third breast pumping session to flow from the nipple to the manifold 29 via the breast cup 2. In some embodiments, the time between second and third pumping sessions is from 30 minutes to 240 minutes (e.g., about 30 minutes to about 45 minutes, about 30 minutes to about 60 minutes, about 30 minutes to about 90 minutes, about 30 minutes to about 120 minutes, about 30 minutes to about 180 minutes, about 45 minutes to about 60 minutes, about 45 minutes to about 90 minutes, about 45 minutes to about 120 minutes, about 60 minutes to about 90 minutes, about 60 minutes to about 120 minutes, about 50 minutes to about 180 minutes, about 60 minutes to about 210 minutes, about 90 minutes to about 120 minutes, about 90 minutes to about 180 minutes, about 90 minutes to about 210 minutes, about 90 minutes to about 240 minutes, about 120 minutes to about 180 minutes, about 120 minutes to about 210 minutes, about 120 minutes to about 240 minutes, about 180 minutes to about 210 minutes, about 180 minutes to about 240 minutes, about 210 minutes to about 240 minutes, about 30 minutes, about 45 minutes, about 60 minutes, about 85 minutes, about 90 minutes, about 105 minutes, about 120 minutes, about 135 minutes, about 150 minutes, about 165 minutes, about 180 minutes, about 195 minutes, about 210 minutes, about 225 minutes, or about 240 minutes).

In some embodiments, the method further includes providing fluidic communication between (i) the inlet 31 of the manifold 29 and (ii) the first reservoir 3, the second reservoir 3, or the third fluid reservoir 3 by moving, e.g., rotating, the moveable, e.g., rotating, member. In some embodiments, the method further includes removing fluidic communication between (i) the inlet of the manifold and (ii) the first reservoir 3, the second reservoir 3, or the third fluid reservoir 3 by moving, e.g., rotating, the moving, e.g., rotating, member. In some embodiments, the moving, e.g., rotating, member rotates clockwise with respect to the stationary member. In some embodiments, the moving, e.g., rotating, member rotates counterclockwise with respect to the stationary member.

In some embodiments, the method further includes providing fluidic communication between (i) the inlet of the manifold 29 and (ii) the first reservoir 3, the second reservoir 3, or the third fluid reservoir 3 by opening the valve to the inlet of the first reservoir 3, the second reservoir 3, or the third fluid reservoir 3. In some embodiments, the method further includes removing fluidic communication between (i) the inlet 30 of the manifold 29 and (ii) the first reservoir 3, the second reservoir 3, or the third fluid reservoir 3 by closing the valve 24 to the inlet of the first reservoir 3, the second reservoir 3, or the third fluid reservoir 3.

In some embodiments, the method further includes providing fluidic communication between (i) the inlet of the manifold 29 and (ii) the first reservoir 3, the second reservoir 3, or the third fluid reservoir 3 by moving the arm. In some embodiments, the method further includes removing fluidic communication between (i) the inlet of the manifold 29 and (ii) the first reservoir 3, the second reservoir 3, or the third fluid reservoir 3 by moving the arm. In some embodiments, the method further includes moving the arm with the drive unit 11, wherein the drive unit 11 is the stepper motor.

In some embodiments, the method further includes turning on the modular breast pump system 1. The modular breast pump system 1 may be turned on after the breast cup 2 is contacted to the human breast. In some embodiments, the modular breast pump system 1 may be turned on before the breast cup 2 is contacted to the human breast, e.g., the negative pressure is applied after the human breast is contacted, or the human breast is contacted during a waiting period before the negative pressure is applied. In some embodiments, modular breast pump system 1 is turned on with the power switch 14. In some embodiments, the modular breast pump system 1 is turned on remotely. In some embodiments, the modular breast pump system 1 is turned on automatically according to a pre-programmed schedule. In some embodiments, the modular breast pump system 1 is turned on automatically according to a pre-programmed schedule a plurality of times.

In some embodiments, the method further includes turning on a pressure source. A pressure source may be turned on after the breast cup 2 contacts the human breast. In some embodiments, the pressure source is turned on with the power switch 14. In some embodiments, the pressure source is turned on remotely. In some embodiments, the pressure source is turned on automatically according to a pre-programmed schedule. In some embodiments, the pressure source is turned on automatically according to a pre-programmed schedule a plurality of times. The pressure source may be a negative pressure source or a positive pressure source.

In some embodiments, the method further includes turning on the pressure source 9. The pressure source 9 may be turned on after the breast cup 2 is contacted to the human breast. In some embodiments, pressure source 9 is turned on with the power switch 14. In some embodiments, the pressure source 9 is turned on remotely. In some embodiments, the pressure source 9 is turned on automatically according to a pre-programmed schedule. In some embodiments, the pressure source 9 is turned on automatically according to a pre-programmed schedule a plurality of times.

The first pressure or second pressure may be between about 0 mmHg to about 250 mmHg, e.g., about 0 mmHg to about 10 mmHg, about 0 mmHg to about 20 mmHg, about 0 mmHg to about 30 mmHg, about 0 mmHg to about 40 mmHg, about 0 mmHg to about 50 mmHg, about 0 mmHg to about 60 mmHg, about 0 mmHg to about 70 mmHg, about 0 mmHg to about 80 mmHg, about 0 mmHg to about 90 mmHg, about 0 to about 100 mmHg, about 0 mmHg to about 110 mmHg, about 0 mmHg to about 120 mmHg, about 0 mmHg to about 130 mmHg, about 0 to about 140 mmHg, about 0 mmHg to about 150 mmHg, about 0 to about 175 mmHg, about 0 mmHg to about 200 mmHg, about 0 mmHg to about 225 mmHg, about 0 mmHg to about 250 mmHg, about 0 mmHg to about 275 mmHg, about 0 mmHg to about 300 mmHg, about 0 to about 350 mmHg, about 0 mmHg to about 400 mmHg, about 10 mmHg to about 20 mmHg, about 10 mmHg to about 30 mmHg, about 10 mmHg to about 40 mmHg, about 10 mmHg to about 50 mmHg, about 10 mmHg to about 60 mmHg, about 10 mmHg to about 70 mmHg, about 10 mmHg to about 80 mmHg, about 10 mmHg to about 90 mmHg, about 10 mmHg to about 100 mmHg, about 10 mmHg to about 110 mmHg, about 10 mmHg to about 120 mmHg, about 10 mmHg to about 130 mmHg, about 10 mmHg to about 140 mmHg, about 10 mmHg to about 150 mmHg, about 10 mmHg to about 175 mmHg, about 10 mmHg to about 200 mmHg, about 10 mmHg to about 225 mmHg, about 10 mmHg to about 250 mmHg, about 10 mmHg to about 300 mmHg, about 10 to about 350 mmHg, about 10 mmHg to about 400 mmHg, about 20 mmHg to about 30 mmHg, about 20 mmHg to about 40 mmHg, about 20 mmHg to about 50 mmHg, about 20 mmHg to about 60 mmHg, about 20 mmHg to about 70 mmHg, about 20 mmHg to about 80 mmHg, about 20 mmHg to about 90 mmHg, about 20 mmHg to about 100 mmHg, about 20 mmHg to about 110 mmHg, about 20 mmHg to about 120 mmHg, about 20 mmHg to about 130 mmHg, about 20 mmHg to about 140 mmHg, about 20 mmHg to about 150 mmHg, about 20 mmHg to about 175 mmHg, about 20 mmHg to about 200 mmHg, about 20 mmHg to about 225 mmHg, about 20 mmHg to about 250 mmHg, about 20 mmHg to about 300 mmHg, about 20 to about 350 mmHg, about 20 mmHg to about 400 mmHg, about 25 mmHg to about 50 mmHg, about 25 mmHg to about 75 mmHg, about 25 mmHg to about 100 mmHg, about 30 mmHg to about 40 mmHg, about 30 mmHg to about 50 mmHg, about 30 mmHg to about 60 mmHg, about 30 mmHg to about 70 mmHg, about 30 mmHg to about 80 mmHg, about 30 mmHg to about 90 mmHg, about 30 mmHg to about 100 mmHg, about 30 mmHg to about 110 mmHg, about 30 mmHg to about 120 mmHg, about 30 mmHg to about 130 mmHg, about 30 mmHg to about 140 mmHg, about 30 mmHg to about 150 mmHg, about 30 mmHg to about 175 mmHg, about 30 mmHg to about 200 mmHg, about 30 mmHg to about 225 mmHg, about 30 mmHg to about 250 mmHg, about 30 mmHg to about 300 mmHg, about 30 to about 350 mmHg, about 30 mmHg to about 400 mmHg, about 40 mmHg to about 50 mmHg, about 40 mmHg to about 60 mmHg, about 40 mmHg to about 70 mmHg, about 40 mmHg to about 80 mmHg, about 40 mmHg to about 90 mmHg, about 40 mmHg to about 100 mmHg, about 40 mmHg to about 110 mmHg, about 40 mmHg to about 120 mmHg, about 40 mmHg to about 130 mmHg, about 40 mmHg to about 140 mmHg, about 40 mmHg to about 150 mmHg, about 40 mmHg to about 175 mmHg, about 40 mmHg to about 200 mmHg, about 40 mmHg to about 225 mmHg, about 40 mmHg to about 250 mmHg, about 40 mmHg to about 300 mmHg, about 40 to about 350 mmHg, about 40 mmHg to about 400 mmHg, about 50 mmHg to about 60 mmHg, about 50 mmHg to about 70 mmHg, about 50 mmHg to about 75 mmHg, about 50 mmHg to about 80 mmHg, about 50 mmHg to about 90 mmHg, about 50 mmHg to about 100 mmHg, about 50 mmHg to about 110 mmHg, about 50 mmHg to about 120 mmHg, about 50 mmHg to about 130 mmHg, about mmHg 50 to about 140 mmHg, about 50 mmHg to about 150 mmHg, about 50 mmHg to about 175 mmHg, about 50 mmHg to about 200 mmHg, about 50 mmHg to about 225 mmHg, about 50 mmHg to about 250 mmHg, about 50 mmHg to about 300 mmHg, about 50 to about 350 mmHg, about 50 mmHg to about 400 mmHg, about 60 mmHg to about 70 mmHg, about 60 mmHg to about 80 mmHg, about 60 mmHg to about 90 mmHg, about 60 mmHg to about 100 mmHg, about 60 mmHg to about 110 mmHg, about 60 mmHg to about 120 mmHg, about 60 mmHg to about 130 mmHg, about 60 mmHg to about 140 mmHg, about 60 mmHg to about 150 mmHg, about 60 mmHg to about 175 mmHg, about 60 mmHg to about 200 mmHg, about 60 mmHg to about 225 mmHg, about 60 mmHg to about 250 mmHg, about 60 mmHg to about 300 mmHg, about 60 to about 350 mmHg, about 60 mmHg to about 400 mmHg, about 70 mmHg to about 80 mmHg, about 70 mmHg to about 90 mmHg, about 70 mmHg to about 100 mmHg, about 70 mmHg to about 110 mmHg, about 70 mmHg to about 120 mmHg, about 70 mmHg to about 130 mmHg, about 70 mmHg to about 140 mmHg, about 70 mmHg to about 150 mmHg, about 70 mmHg to about 175 mmHg, about 70 mmHg to about 200 mmHg, about 70 mmHg to about 225 mmHg, about 70 mmHg to about 250 mmHg, about 70 mmHg to about 300 mmHg, about 70 to about 350 mmHg, about 70 mmHg to about 400 mmHg, about 75 mmHg to about 100 mmHg, about 75 mmHg to about 125 mmHg, about 80 mmHg to about 90 mmHg, about 80 mmHg to about 100 mmHg, about 80 mmHg to about 110 mmHg, about 80 mmHg to about 120 mmHg, about 80 mmHg to about 130 mmHg, about 80 mmHg to about 140 mmHg, about 80 mmHg to about 150 mmHg, about 80 mmHg to about 175 mmHg, about 80 mmHg to about 200 mmHg, about 80 mmHg to about 225 mmHg, about 80 mmHg to about 250 mmHg, about 80 mmHg to about 300 mmHg, about 80 to about 350 mmHg, about 80 mmHg to about 400 mmHg, about 90 mmHg to about 100 mmHg, about 90 mmHg to about 110 mmHg, about 90 mmHg to about 120 mmHg, about 90 mmHg to about 130 mmHg, about 90 mmHg to about 140 mmHg, about 90 mmHg to about 150 mmHg, about 90 mmHg to about 175 mmHg, about 90 mmHg to about 200 mmHg, about 90 mmHg to about 225 mmHg, about 90 mmHg to about 250 mmHg, about 90 mmHg to about 300 mmHg, about 90 to about 350 mmHg, about 90 mmHg to about 400 mmHg, about 100 mmHg to about 110 mmHg, about 100 mmHg to about 120 mmHg, about 100 mmHg to about 130 mmHg, about 100 mmHg to about 140 mmHg, about 100 mmHg to about 150 mmHg, about 100 mmHg to about 175 mmHg, about 100 mmHg to about 200 mmHg, about 100 mmHg to about 225 mmHg, about 100 mmHg to about 250 mmHg, about 100 mmHg to about 300 mmHg, about 100 to about 350 mmHg, about 100 mmHg to about 400 mmHg, about 110 mmHg to about 120 mmHg, about 110 mmHg to about 130 mmHg, about 110 mmHg to about 140 mmHg, about 110 mmHg to about 150 mmHg, about 110 mmHg to about 175 mmHg, about 110 mmHg to about 200 mmHg, about 110 mmHg to about 225 mmHg, about 110 mmHg to about 250 mmHg, about 110 mmHg to about 300 mmHg, about 110 to about 350 mmHg, about 110 mmHg to about 400 mmHg, about 120 mmHg to about 130 mmHg, about 120 mmHg to about 140 mmHg, about 120 mmHg to about 150 mmHg, about 120 mmHg to about 175 mmHg, about 120 mmHg to about 200 mmHg, about 120 mmHg to about 225 mmHg, about 120 mmHg to about 250 mmHg, about 120 mmHg to about 300 mmHg, about 120 to about 350 mmHg, about 120 mmHg to about 400 mmHg, about 130 mmHg to about 140 mmHg, about 130 mmHg to about 150 mmHg, about 130 mmHg to about 175 mmHg, about 130 mmHg to about 200 mmHg, about 130 mmHg to about 225 mmHg, about 130 mmHg to about 250 mmHg, about 130 mmHg to about 300 mmHg, about 130 to about 350 mmHg, about 130 mmHg to about 400 mmHg, about 140 mmHg to about 150 mmHg, about 140 mmHg to about 175 mmHg, about 140 mmHg to about 200 mmHg, about 140 mmHg to about 225 mmHg, about 140 mmHg to about 250 mmHg, about 140 mmHg to about 300 mmHg, about 140 to about 350 mmHg, about 140 mmHg to about 400 mmHg, about 150 mmHg to about 175 mmHg, about 150 mmHg to about 200 mmHg, about 150 mmHg to about 225 mmHg, about 150 mmHg to about 250 mmHg, about 150 mmHg to about 300 mmHg, about 150 to about 350 mmHg, about 150 mmHg to about 400 mmHg, about 175 mmHg to about 200 mmHg, about 175 mmHg to about 225 mmHg, about 175 mmHg to about 250 mmHg, about 175 mmHg to about 300 mmHg, about 175 to about 350 mmHg, about 175 mmHg to about 400 mmHg, about 200 mmHg to about 225 mmHg, about 200 mmHg to about 250 mmHg, about 200 mmHg to about 300 mmHg, about 200 to about 350 mmHg, about 200 mmHg to about 400 mmHg, about 225 mmHg to about 250 mmHg, about 225 mmHg to about 300 mmHg, about 225 to about 350 mmHg, about 225 mmHg to about 400 mmHg, about 275 mmHg to about 300 mmHg, about 275 to about 350 mmHg, about 275 mmHg to about 400 mmHg, about 300 mmHg to about 325 mmHg, about 300 to about 350 mmHg, about 300 mmHg to about 400 mmHg, about 325 mmHg to about 350 mmHg, about 325 to about 375 mmHg, about 325 mmHg to about 400 mmHg, about 350 mmHg to about 375 mmHg, about 350 to about 400 mmHg, or about 375 mmHg to about 400 mmHg.

The modular breast pump system 1 may pump from about 1 second to about 2 hours, about e.g., about 30 seconds to about 1 minute, about 30 seconds to about 2 minutes, about 30 seconds to about 3 minutes, about 30 seconds to about 4 minutes, about 30 seconds to about 5 minutes, about 30 seconds to about 10 minutes, about 30 seconds to about 15 minutes, about 30 seconds to about 30 minutes, about 30 seconds to about 5 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 35 minutes, about 5 minutes to about 40 minutes, about 5 minutes to about 1 hour, about 10 minutes to about 15 minutes, about 10 minutes to about 20 minutes, about 10 minutes to about 25 minutes, about 10 minutes to about 30 minutes, about 10 minutes to about 40 minutes, about 10 minutes to about 50 minutes, about 10 minutes to about 1 hour, about 15 minutes to about 30 minutes, about 15 minutes to about 45 minutes, about 15 minutes to about 1 hour, about 15 minutes to about 1.25 hours, about 15 minutes to about 1.5 hours, about 15 minutes to about 1.75 hours, about 15 minutes to about 2 hours, about 20 minutes to about 30 minutes, about 20 minutes to about 40 minutes, about 20 minutes to about 50 minutes, about 30 minutes to about 45 minutes, about 30 minutes to about 1 hour, about 30 minutes to about 1.25 hours, about 30 minutes to about 1.5 hours, about 30 minutes to about 1.75 hours, about 30 minutes to about 2 hours, about 45 minutes to about 1 hour, about 45 minutes to about 1.25 hours, about 45 minutes to about 1.5 hours, about 45 minutes to about 1.75 hours, about 45 minutes to about 2 hours, about 1 hour to about 1.25 hours, about 1 hour to about 1.5 hours, about 1 hour to about 1.75 hours, about 1 hour to about 2 hours, about 1.25 hours to about 1.5 hours, about 1.25 hours to about 1.75 hours, about 1.25 hours to about 2 hours, about 1.5 hours to about 1.75 hours, about 1.5 hours to about 2 hours, or about 1.75 hours to about 2 hours. The user may input a pump program from about 1 second to about 2 hours, and, e.g., set the program before the start of an activity, rest, or sleep.

In some embodiments, the method further includes turning on a positive pressure source. A positive pressure source may be turned on after the breast cup 2 contacts the human breast. In some embodiments, the positive pressure source is turned on with the power switch 14. In some embodiments, the positive pressure source is turned on remotely. In some embodiments, the positive pressure source is turned on automatically according to a pre-programmed schedule. In some embodiments, the positive pressure source is turned on automatically according to a pre-programmed schedule a plurality of times.

In some embodiments, the method further includes turning off the modular breast pump system 1. In some embodiments, the modular breast pump system 1 is turned off with the power switch 14. In some embodiments, the modular breast pump system 1 is turned off remotely. In some embodiments, the modular breast pump system 1 is turned off automatically according to a pre-programmed schedule. In some embodiments, the modular breast pump system 1 is turned off automatically according to a pre-programmed schedule a plurality of times.

In some embodiments, the method further includes turning off the pressure source 9. In some embodiments, the pressure source 9 is turned off with the power switch 14. In some embodiments, the pressure source 9 is turned off remotely. In some embodiments, the pressure source 9 is turned off automatically according to a pre-programmed schedule. In some embodiments, the pressure source 9 is turned off automatically according to a pre-programmed schedule a plurality of times.

The method may include alternating between a first pressure to extend the nipple and express milk, and a second pressure to contact the nipple and slow or stop milk expression. Additionally, the method may include pumping at from two to ten pressures, e.g., one, two, three, four, five, six, seven, eight, nine, or ten pressures. The pressures applied to the breast may increase or decrease throughout the pumping.

In some embodiments, the method includes placing an ice pack in the enclosure 4. In some embodiments, the method includes removing the ice pack from the enclosure 4. In some embodiments, the method includes placing the ice pack in a freezer.

As discussed above, the modular breast pump system 1 may include at least one sensor. The method may further include the taking of a weight, temperature, pressure, pH, viscosity, optical, volume, or flow measurement. The method may further include alerting a user of a sensor measurement. The method may further include changing at least one of the first pressure or second pressure based on the temperature, weight, pressure, or flow measurement.

The present disclosure provides methods of breast pumping while the user is in motion. Further, the present disclosure allows for methods to allow a mother or other care provider to carry out pumping in accordance with lifestyle preferences.

For example, speaking generally, if a new mother or other care provider wishes to resume activities requiring travel (e.g., to work) as part of their daily routine, the possibility of not initiating or prematurely ending the breastfeeding period may increase. The present disclosure provides modular breast pump systems 1 which may be used while the user is in motion, such that the user may consistently breast pump while fulfilling other activities (e.g., travel to and from work and being outside of the home) associated with their daily routine. The present disclosure further provides modular breast pump systems 1 which may be used while the user is working.

The present disclosure provides methods of breast pumping while resting, sleeping, or traveling. As will be understood, the user may be seated or otherwise not actively moving while pumping in a location away from home.

For example, while attempting to sleep, the possibility of not initiating or prematurely ending the breastfeeding period may increase. It has been found that nearly 75% of sleep disruption in mothers was caused by breastfeeding. The present disclosure provides modular breast pump systems 1 which may be used during rest or sleep, thus allowing mothers to pump for a longer time post-birth with reduced interruption of sleep.

In some embodiments, the breast cup 2 includes a wide portion and a nipple tunnel, and the method includes contacting the human breast with the wide portion of the breast cup 2. In some embodiments, the method includes positioning the nipple in the nipple tunnel of the breast cup 2. In some embodiments, the method includes contacting two human breasts with the two breast cups 2.

In some embodiments, the method includes applying negative pressure to the pressure chamber 20 with the pressure source 9. Pressure around the nipple is reduced to below atmospheric pressure.

In some embodiments, the method includes deforming the diaphragm 18 away from the breast cup. In some embodiments, the method includes deforming the diaphragm 18 away from the inlet. In some embodiments, the method includes unsealing the inlet. Deformation of the diaphragm 18 away from the inlet allows milk to flow from the nipple to the milk chamber 21. In some embodiments, the method includes expanding the milk chamber 21. In some embodiments, the method includes providing negative pressure to elongate the nipple. In some embodiments, the method includes drawing milk from the nipple. In some embodiments, the method includes drawing milk from the nipple tunnel 2 into the milk chamber 21. In some embodiments, the method includes drawing milk through the inlet.

In some embodiments, the method includes opening the control valve 10 and depressurizing the pressure chamber 20. In some embodiments, the method includes returning the diaphragm 18 to a nominal position. In some embodiments, the method includes closing the control valve 10.

In some embodiments, the method includes applying positive pressure to the pressure chamber 21 with the positive pressure source. In some embodiments, the method includes allowing the nipple to contract. In some embodiments, the method includes deforming the diaphragm 18 towards the breast shield 19. In some embodiments, the method includes allowing the diaphragm 18 to return to a nominal position. In some embodiments, the method includes sealing the inlet with the diaphragm 18. Deformation of the diaphragm 18 towards the breast shield seals the inlet such that the nipple is allowed to contract. In some embodiments, the method includes sealing the orifices with diaphragm 18. In some embodiments, the method includes contracting the milk chamber 21. Contraction of the milk chamber acts to pump milk out of the breast cup.

In some embodiments, the method includes transporting milk from the milk chamber 21 to the reservoir 3. In some embodiments, the method includes transporting milk through the first fluid conduit. In some embodiments, the method includes transporting milk through the outlet. In some embodiments, the method includes transporting milk through the one directional valve. In some embodiments, the method includes applying pressure to the manifold 29 with a pressure source to transport milk to a reservoir 3. Thus, in some embodiments, the pressure source, or a second pressure source, can be in fluidic communication with the manifold 29. In some embodiments, a positive pressure source is in fluidic communication with the manifold 29 and transports milk to a reservoir 3. In some embodiments, the method includes applying a negative pressure source to the milk chamber 21 at the end of the pumping session. In this embodiment, the negative pressure transports any residual milk from the milk chamber 21 and fluid conduit 5 into the reservoir 3. In some embodiments, the negative pressure source is a peristaltic pump, e.g., on a first conduit or downstream component connected thereto. Positive pressure may also be introduced into the milk chamber, e.g., via a separate inlet, to aid in transport of milk or cleaning. The clearing of milk from the fluid conduit and/or milk chamber may occur only once at the end of a breast pumping cycle.

The present disclosure provides a method of distributing milk from breast pumping using the base and portable modules while mated, including: (i) providing a base module including a milk distribution system 28 of the present disclosure; (ii) pumping a human breast to induce milk from a first breast pumping session to flow from a nipple to the manifold 29 via the breast cup 2; (iii) providing fluidic communication between the inlet 30 of the manifold 29 and a first reservoir 3 to allow milk from the first breast pumping session to flow into the first reservoir 3; (iv) removing fluidic communication between the inlet 30 of the manifold 29 and the first fluid reservoir 3; (v) pumping the human breast to induce milk from a second breast pumping session to flow from the nipple to the manifold 29 via the breast cup 2; and (vi) providing fluidic communication between the inlet 30 of the manifold 29 and a second reservoir 3 to allow milk from the second breast pumping session to flow into the second reservoir 3.

In some embodiments, providing fluidic communication between the inlet 30 of the manifold 29 and the first reservoir 3 includes opening a first valve 24. In some embodiments, the control unit sends a signal to the first valve 24 to open and/or close. In some embodiments, the control unit sends a signal to the second valve 24 to open and/or close.

In some embodiments, the time between step (ii) and step (v) is from about 30 minutes to about 240 minutes (e.g., 30 minutes to 60 minutes, 30 minutes to 90 minutes, 60 minutes to 90 minutes, about 60 minutes to about 120 minutes, about 60 minutes to about 180 minutes, about 90 minutes to about 180 minutes, about 90 minutes to about 240 minutes, or about 120 minutes to about 240 minutes).

In some embodiments, the method further includes (vi) removing fluidic communication between the inlet 30 of the manifold 29 and the second fluid reservoir 3; and (vii) providing fluidic communication between the inlet 30 of the manifold 29 and a third reservoir 3; and (viii) pumping the human breast to induce milk from a third breast pumping session to flow from the nipple to the manifold 29 via the breast cup 2. In some embodiments, the time between step (v) and step (viii) is from 30 minutes to 240 minutes.

In some embodiments, the method further includes providing fluidic communication between (i) the inlet 30 of the manifold 29 and (ii) the first reservoir 3, the second reservoir 3, or the third fluid reservoir 3 by moving, e.g., rotating, the moving, e.g., rotating, member. In some embodiments, the method further includes removing fluidic communication between (i) the inlet 30 of the manifold 29 and (ii) the first reservoir 3, the second reservoir 3, or the third fluid reservoir 3 by moving, e.g., rotating, the moving, e.g., rotating, member.

In some embodiments, the method further includes providing fluidic communication between (i) the inlet 30 of the manifold 29 and (ii) the first reservoir 3, the second reservoir 3, or the third fluid reservoir 3 by opening the valve 24 to the inlet of the first reservoir 3, the second reservoir 3, or the third fluid reservoir 3. In some embodiments, the method further includes removing fluidic communication between (i) the inlet 30 of the manifold 29 and (ii) the first reservoir 3, the second reservoir 3, or the third fluid reservoir 3 by closing the valve 24 to the inlet of the first reservoir 3, the second reservoir 3, or the third fluid reservoir 3.

In some embodiments, the method further includes providing fluidic communication between (i) the inlet 30 of the manifold 29 and (ii) the first reservoir 3, the second reservoir 3, or the third fluid reservoir 3 by moving the arm. In some embodiments, the method further includes removing fluidic communication between (i) the inlet 30 of the manifold 29 and (ii) the first reservoir 3, the second reservoir 3, or the third fluid reservoir 3 by moving the arm. In some embodiments, the method further includes moving the arm with the drive unit 11, wherein the drive unit 11 is the stepper motor.

The disclosure further provides methods of cleaning a modular breast pump system, including providing the modular breast pump system 1, providing a cleaning fluid to breast cup 2; and pumping the cleaning fluid from the breast cup 2 to the reservoir 3 or waste.

In some embodiments, the method includes providing a cleaning fluid to the modular breast pump system 1, e.g., the base module, portable module, and/or breast cups. In some embodiments, the method includes transporting a cleaning fluid from the breast cup 2 to a waste reservoir 3, e.g., in the base and/or portable module. In some embodiments, the cleaning fluid is water. In some embodiments, the method includes pumping milk from the human breast twice, in which there is at least 30 minutes from a first breast pumping session to a second breast pumping session. In some embodiments, the method includes pumping milk from the human breast 1, 2, 3, 4, 5, 6, 7, 8, 9, or 10 times. In some embodiments, the method includes pumping milk from the human breast a plurality of times (e.g., from 2 to 3 times, from 2 to 4 times, from 2 to 5 times, from 2 to 6 times, from 2 to 10 times, from 3 to 4 times, from 3 to 5 times, from 3 to 10 times, from 4 to 5 times, from 4 to 6 times, or from 5 to 10 times). In some embodiments, the method is used to pump breast milk about 8-10 times in 24 hours. Cleaning may occur once every 24 hours, e.g., after about 8-10 pumping sessions.

In some embodiments, a subject including the human breast is moving, reclining, resting, sleeping, or traveling.

### Kits

In another aspect, the disclosure provides kits. Kits may include two or more unconnected components of a modular breast pump system or a breast cup. A kit may include a base module and an portable module or components thereof. A kit may include a base module, an portable module, or components thereof, and one or two breast cups. A kit may include a base module, an portable module, or components thereof, and one or more fluidic conduits. A kit may include a breast cup or components thereof and one or more fluidic conduits. For example, a kit may include two breast cups or components thereof and two or more fluidic conduits. The fluidic conduits, e.g., for the pressure chamber and/or the milk chamber, may be branched to allow connection to both breast cups with a single connection to the pressure source or reservoir or milk distribution system. A kit including components of a breast cup may include at least two of the following components, e.g., all: the upper housing 17, the diaphragm 18, the breast shield 19, and the milk chamber wall 26. A user may assemble the breast cup from the kit. A kit may include two or more components of the modular breast pump system, e.g., one or more breast cups, a base module, an portable module, and a reservoir.

### Alternative Base Module and Portable Module

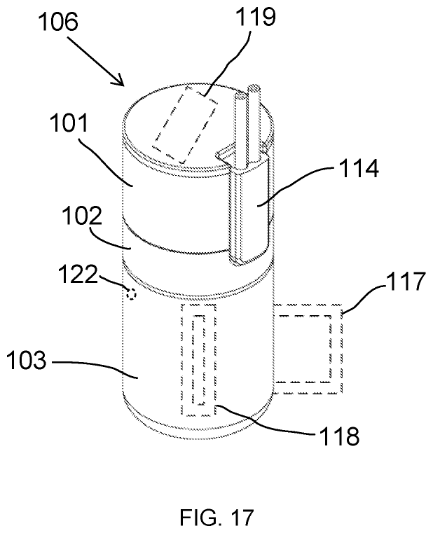

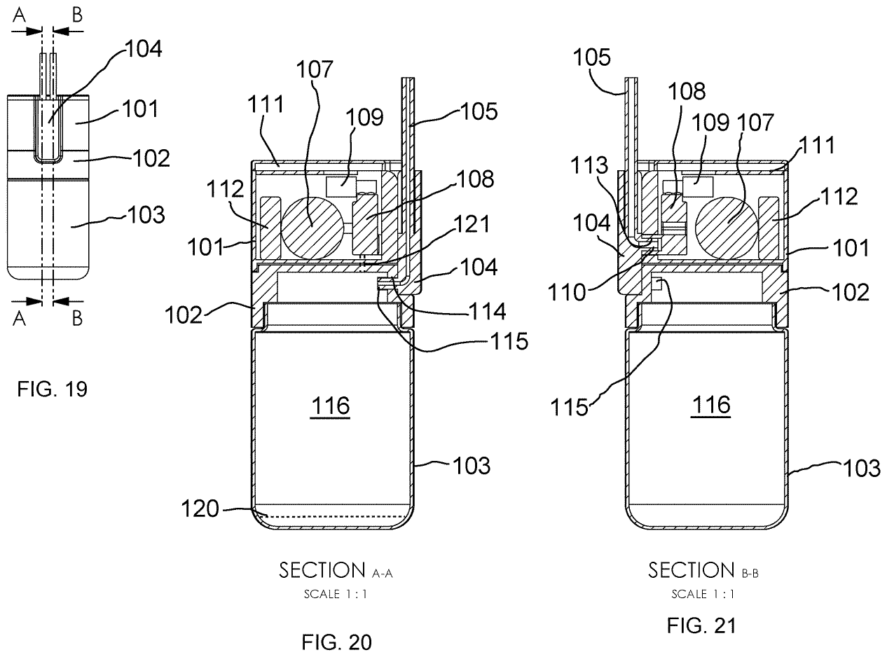

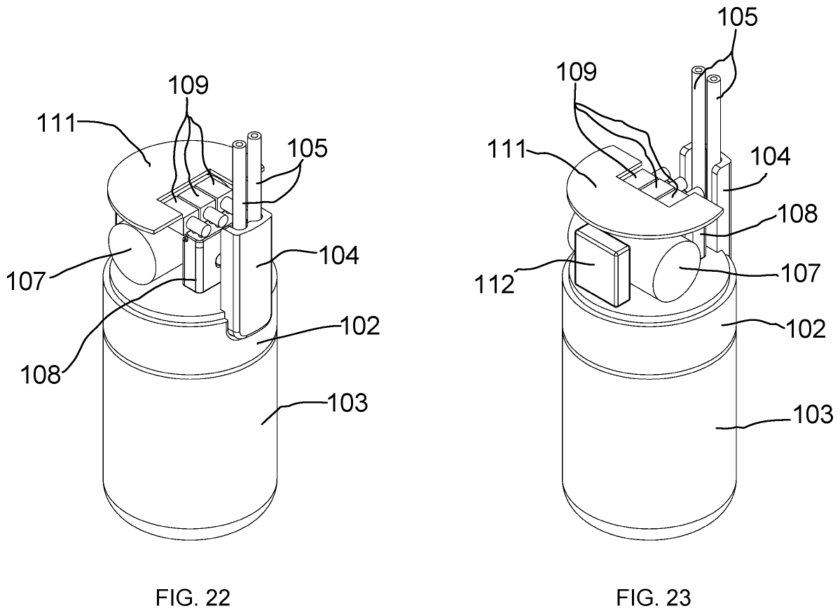

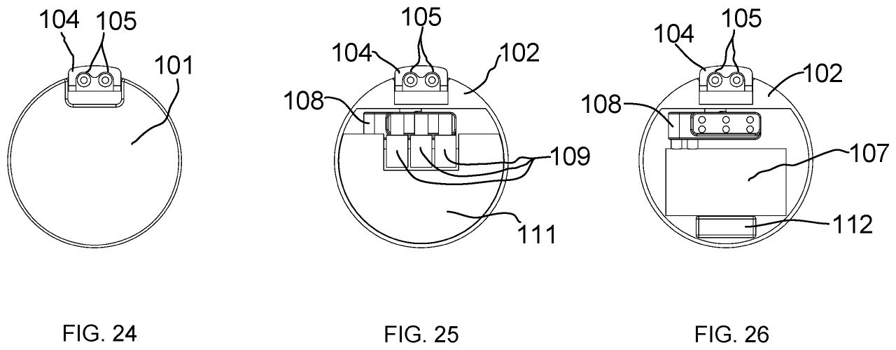

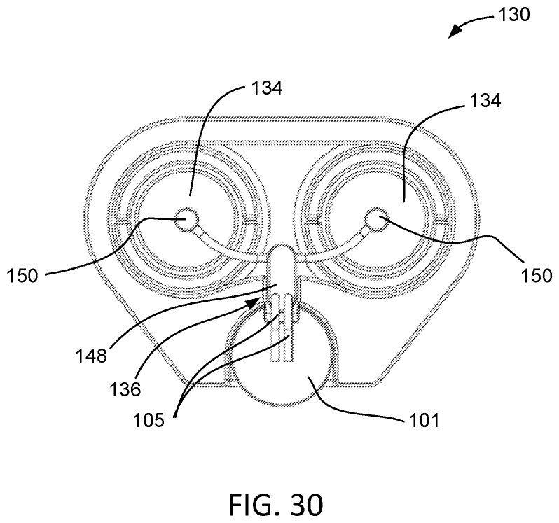

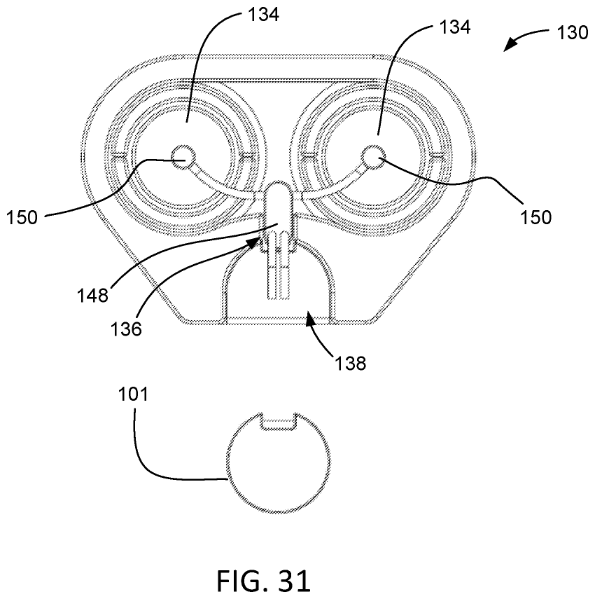

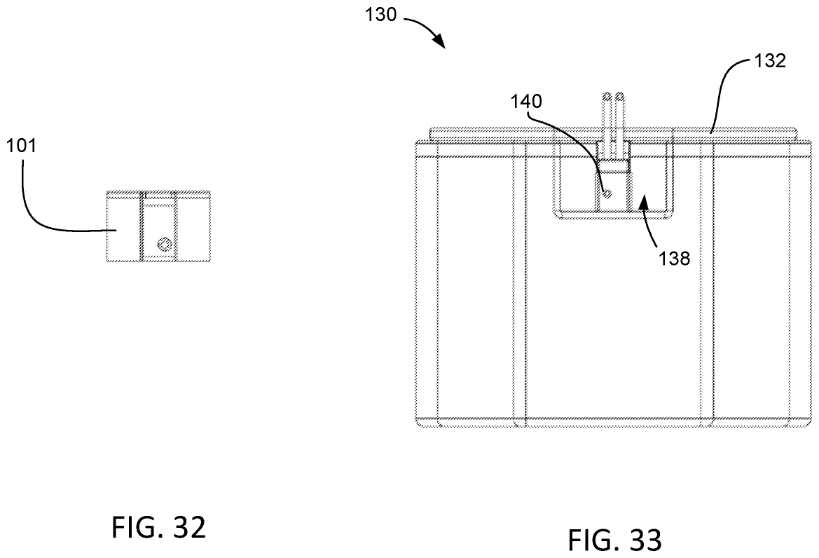

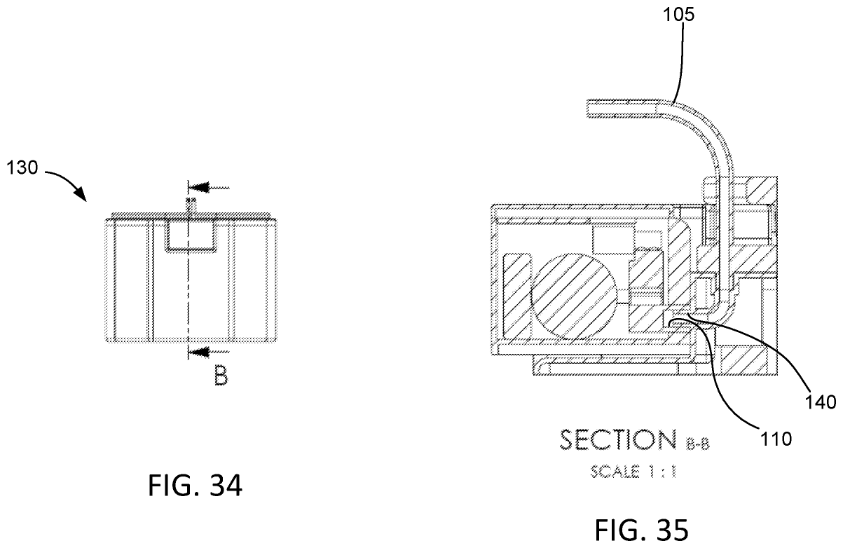

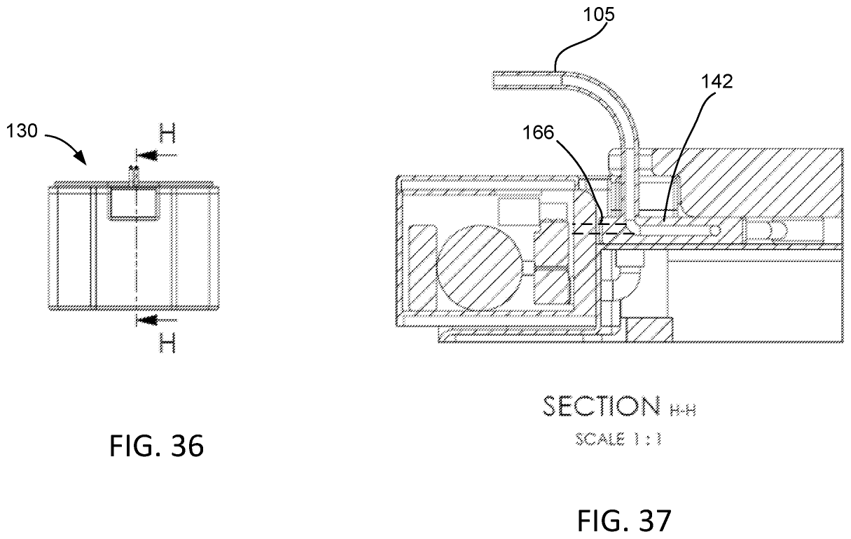

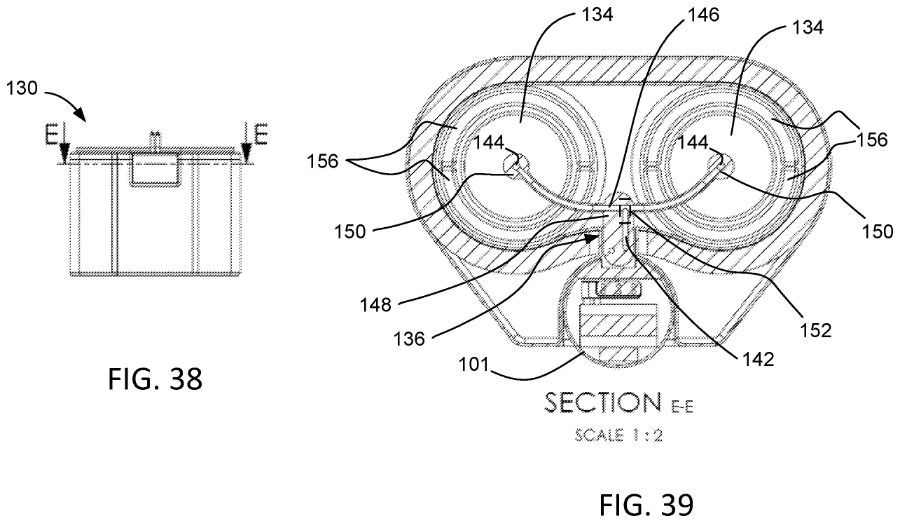

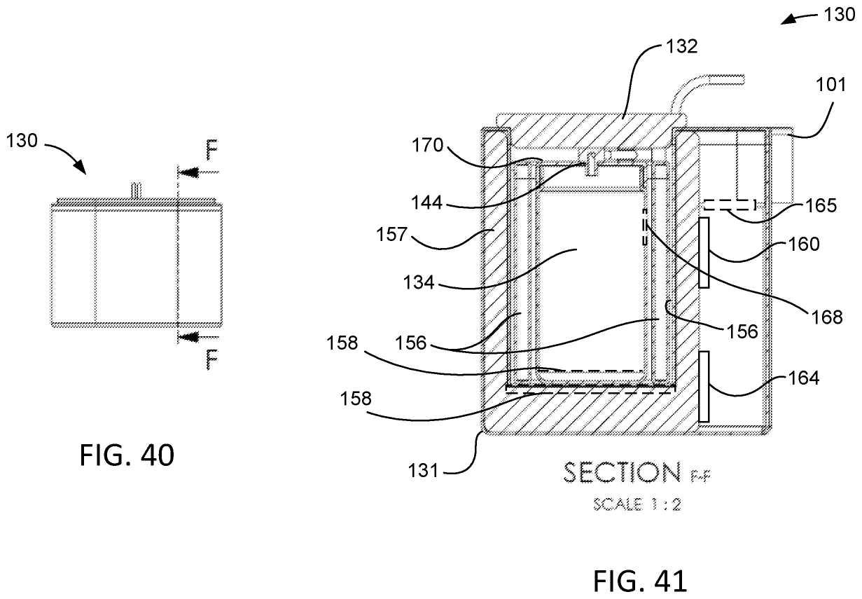

Referring now to FIGS. 17-40, an alternative portable module 106 and base module 130 for use with the modular breast pump system 1 are shown, according to an example embodiment. In some embodiments, the portable module 106 and the base module 130 may each be configured to receive and utilize a removable pumping unit 101 that may be moved back and forth between the portable module 106 and the base module 130 to enable pumping by the corresponding module.

It should be appreciated that the portable module 106 and base module 130 may include any of the various components and/or functionalities of the portable module 6 and base module 7 and may similarly be used in the methods described above. For example, the portable module 106 and the base module 130 are each similarly configured to be used with, connected to (e.g., fluidly coupled to), and/or otherwise adapted to function with the breast cups 2, reservoir enclosures 4 (e.g., the separate reservoir enclosure 4 shown in FIG. 2, and/or any other suitable components of the modular breast pump system 1 described herein. Similarly, any of the various components and/or functionalities of the portable module 106 and the base module 130 described below may be applied to the portable module 6 and the base module 7 described above. Each of these variations is contemplated herein and is within the scope of the present disclosure.

### Portable Module

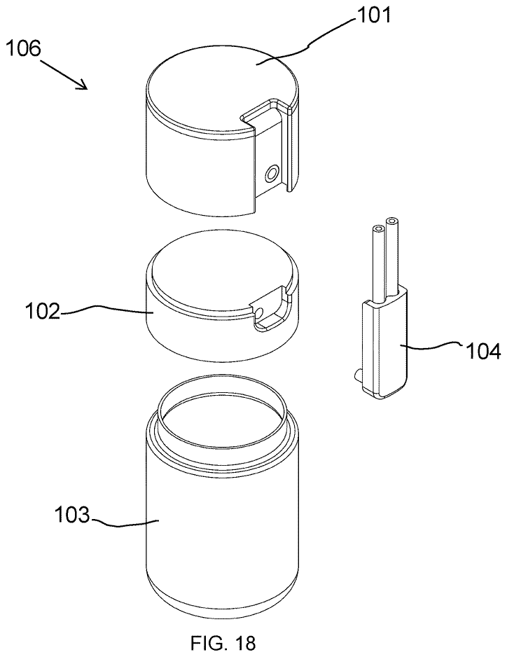

As best illustrated in FIGS. 17 and 18, the portable module 106 is shown with the removable pumping unit 101 installed therein. Accordingly, the portable module 106 includes the removable pumping unit 101, a portable reservoir adapter 102, a portable reservoir 103, and a portable module coupler 104. The removable pumping unit 101 is configured to selectively provide positive and/or negative pressure to the breast cup 2 to induce lactation and, ultimately, force lactated milk from the breast cup 2 into the portable reservoir 103 via fluid conduit lines 105. It will be understood that the fluid conduit lines 105 may be substantially similar or the same as the fluid conduit lines 5 discussed above, and may be used in combination with or instead of the fluid conduit lines 5. In some instances, the fluid conduit lines 105 are the only thing coupling the portable module 106 to the breast cup 2.

For example, as shown in FIGS. 20 and 21, the removable pumping unit 101 includes a pressure source 107 (e.g., a pump) fluidly coupled to a manifold 108 (e.g., via various fluid conduit lines). The manifold 108 includes a number of solenoid valves 109 configured to selectively direct positive and negative pressure from the pressure source 107 through a pumping unit outlet 110. The pumping unit outlet 110 is configured to receive a pumping unit port 113 of the portable module coupler 104 to fluidly couple the pumping unit outlet 110 to the pressure chamber 20 of the breast cup 2 via a fluid conduit line 105.

The removable pumping unit 101 further includes a processing circuit or controller 111 configured to selectively activate and actuate the pressure source 107 and the solenoid valves 109 to selectively direct the positive and negative pressure from the pressure source 107 to the breast cup 2. For example, the processing circuit or controller 111 may include one or more processors and one or more memories storing instructions thereon that, when executed by the one or more processors, cause the one or more processing circuits to perform the various functions described herein.

In some instances, the processing circuit or controller 111 may further include one of more communication interfaces configured to allow for the processing circuit or controller 111 to communicate with other devices over a communications network (e.g., Bluetooth, Wi-Fi, near-field communication, etc.). Additionally, in some instances, the processing circuit or controller 111 may include one or more input/output (I/O) devices 119 (shown in FIG. 17) configured to allow for user to selectively operate the removable pumping unit 101 and/or modify functionality parameters of the removable pumping unit 101. For example, the one or more I/O devices may include a display, one or more buttons (e.g., a power button, a pumping session start/stop button), a touchscreen, an audio speaker, a timer, an alarm, a power switch, and/or any other suitable I/O devices.

The removable pumping unit 101 further includes a power source 112 (e.g., a battery). In some instances, the power source 112 may be rechargeable via connection with an external power source (e.g., a wall outlet). In some other instances, the removable pumping unit 101 may be configured to allow for removal and replacement of the power source 112 upon depletion of its power.

In some embodiments, the removable pumping unit 101 is removably coupled to the portable reservoir adapter 102 via any of a threaded connection, a snap-fit connection, a latch-based connection, or any other suitable releasable connection method. The portable reservoir adapter 102 includes a reservoir inlet 114 configured to receive a reservoir port 115 of the portable module coupler 104 to fluidly couple the reservoir inlet 114 (and thus an internal cavity 116 formed within and between the portable reservoir adapter 102 and the portable reservoir 103) to the milk chamber 21 of the breast cup 2 via a fluid conduit line 105.

In some embodiments, the portable reservoir adapter 102 is removably coupled to the portable reservoir 103 via any of a threaded connection, a snap-fit connection, a latch-based connection, or any other suitable releasable connection method. Further, while shown as separate components, in some instances, the portable reservoir adapter 102 and the portable reservoir 103 may be formed as a single integrated component.

The portable module coupler 104 is configured to releasably and fluidly couple the pumping unit outlet 110 and the portable reservoir inlet 114 of the portable reservoir adapter 102 to the fluid conduit lines 105 coupled to the breast cup 2. That is, with the removable pumping unit 101 coupled to the portable reservoir adapter 102, and the portable reservoir adapter 102 coupled to the portable reservoir 103, the portable module coupler 104 is configured to be attached between the removable pumping unit 101 and the portable reservoir adapter 102 by inserting the pumping unit port 113 into the pumping unit outlet 110 and by inserting the reservoir port 115 into the portable reservoir inlet 114. With the portable module coupler 104 attached between the removable pumping unit 101 and the portable reservoir adapter 102, pressure may be selectively applied by the removable pumping unit 101 (e.g., the pressure source 107) to the pressure chamber 20 of the breast cup 2 to induce lactation from the human breast, as described herein. Further, milk pulled into the milk chamber 21, as described herein, is able to flow from or is otherwise forced out of the milk chamber 21 and into the portable reservoir 103. In some instances, to allow for multiple pumping sessions, the portable reservoir 103 may be removed and replaced with a second portable reservoir 103 that is similar or identical to the portable reservoir 103.

In some instances, the portable module 106 may include one or more carrying features. For example in some instances, the portable module 106 may include a handle 117 for holding and/or carrying the portable module 106. Similarly, in some instances, the portable module 106 may additionally or alternatively include an attachment feature 118, such as a clip, a strap, a hook, a zipper, a magnet, a pin, a button, a snap, a hook and loop fastener (e.g., VELCRO®), or a combination thereof, may be attached to the portable module 106, such that the portable module 106 may be releasably attached, e.g., clipped to the clothing (e.g., a belt, a bra) or other items (e.g., a backpack) of the user.

In some instances, the portable module 106 may further include one or more sensors 120. For example, in some instances, one or more sensors 120 are within the portable reservoir 103 (e.g., arranged near or at a bottom of the portable reservoir 103) that are configured to communicate various sensor data captured within the portable reservoir 103 to the processing circuit or controller 111 via a wired or wireless connection. For example, in some instances, the one or more sensors 120 are configured to detect, sense, or otherwise measure one or more of a weight, a temperature, a pH, a viscosity, optical characteristics, or any other relevant information regarding milk stored within the portable reservoir 103. In some other instances, the portable module 106 may include one or more additional sensors located in other portions of the portable module 106 (e.g., within the manifold 108, within the pumping unit outlet 110, within the portable reservoir inlet 114) that are configured to sense, detect, or otherwise measure the same or additional operational characteristics, such as a pressure being produced by the pressure source 107, a volume and/or flowrate of milk flowing into the portable reservoir, and/or any other desired information pertaining to the portable module 106.

Accordingly, to operate the portable module 106 of the modular breast pump system 1, the removable pumping unit 101 may first be attached to the portable reservoir adapter 102, and the portable module coupler 104 may be connected to both the removable pumping unit 101 and the portable reservoir adapter 102, as discussed above. The removable pumping unit 101 may then be activated to apply alternating pressure (e.g., alternating negatives and nominal pressure or alternating negative and positive pressures) to the pressure chamber 20 of the breast cup 2. The alternating pressures applied to the pressure chamber 20 are configured to deform the diaphragm 18 to alternatingly (i) expand the milk chamber 21 and unseal the sealing point 23 around the inlet to the milk chamber 21 to allow milk expressed from the human breast to flow into the milk chamber and (ii) contract the milk chamber 21 and seal the sealing point 23 around the inlet to the milk chamber 21 such that the expressed milk within the milk chamber 21 is forced out of the breast cup 2, through fluid conduit lines (e.g., fluid conduit lines 105), and ultimately into the portable reservoir 103.

It should be appreciated that, unlike traditional breast pumps, the milk may be collected within the portable reservoir 103 of the portable module 106 while the portable module 106 is located away from the breast cup 2. That is, the portable module 106 is connected to the breast cup 2 via fluid conduit lines (e.g., fluid conduit lines 5 or fluid conduit lines 105) that extend a distance away from the breast cup 2 (e.g., at least a foot, at least two feet, at least three feet, at least four feet) to allow for the milk to be collected off of the user's body. This off-body milk collection provides three distinct benefits over traditional systems: (1) the size of the breast cup 2 may be significantly reduced compared to a system in which a reservoir is attached to or otherwise integrated within the breast cup to allow for on-body milk storage; (2) the milk can be removed from the reservoir 103 without needing to remove the breast cup 2 from the user's breast or even needing to access the user's shirt or bra; and (3) noise created by the pressure source 107 is not emitted from within the user's shirt or bra (e.g., it is instead emitted from an off-body location where the portable module 106 or the base module 130 is located). These features allow for the user to more privately and inconspicuously pump and access pumped milk while in public.

In some instances, the diaphragm 18 returning to a nominal state may provide sufficient force onto the milk within the milk chamber 21 to force the milk out of the milk chamber 21 and into the portable reservoir 103. However, in some instances, the removable pumping unit 101 may apply positive pressure to the pressure chamber 20 to further contract the milk chamber 21, and thereby force the milk out of the milk chamber 21 and into the portable reservoir 103. In some instances, negative pressure may be applied from the pressure source 107 via another fluid conduit 121 in the removable pumping unit 101 and portable module coupler 104 to the milk chamber 21 to pull or suction the milk out of the milk chamber 21. However, to prevent a direct connection between the dry side and wet side of the pump system, a membrane diaphragm or another similar device may be utilized to apply the negative pressure from the pressure source 107 to the milk chamber 21.

In some instances, the portable reservoir 103 may include a pressure outlet 122 (e.g., a pressure relieve valve) configured to allow pressurized air to escape from within the portable reservoir 103 to prevent excessive pressure build-up and/or to allow for milk to more easily flow into the portable reservoir 103.

In some instances, the user may then remove the portable reservoir 103 from the portable module 106 to access the stored milk without removing the breast cup 2 from their breast. For example, the user may simply unscrew or otherwise decouple the portable reservoir 103 from the portable reservoir adapter 102 to access the milk stored within the portable reservoir 103 while the breast cup 2 is still attached to the breast of the user.

### Base Module

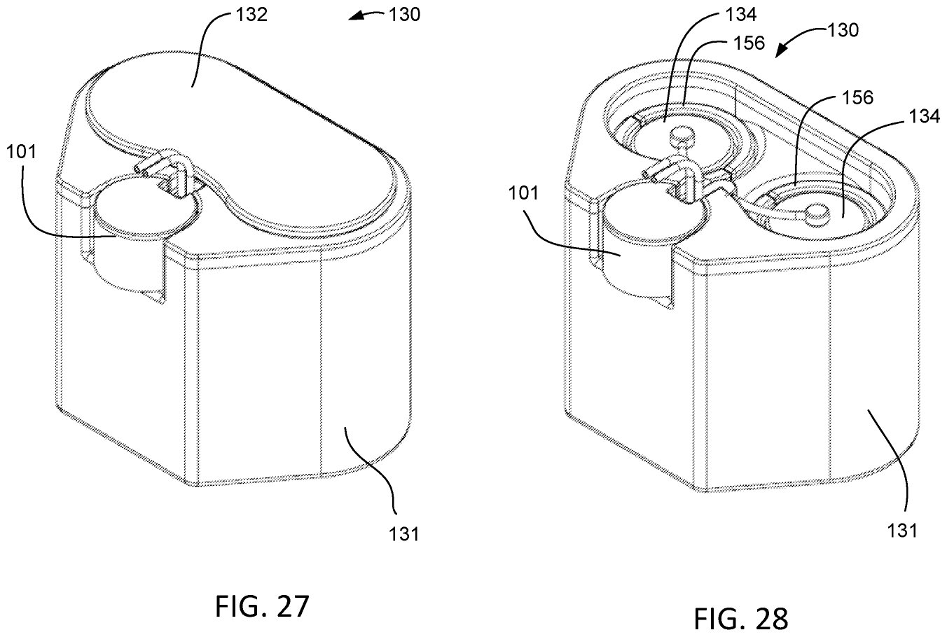

Turning now to the base module 130, as best illustrated in FIGS. 27-28, the removable pumping unit 101 is shown installed within the base module 130. As best shown in FIGS. 27-29, the base module 130 includes the removable pumping unit 101, a reservoir enclosure 131 having a lid 132, removable reservoirs 134 arranged within the reservoir enclosure 131, and a base module coupler 136. The removable pumping unit 101 is similarly configured to selectively provide positive and/or negative pressure to the breast cup 2 to induce lactation and, ultimately, force lactated milk from the breast cup 2 into the removable reservoir 134 via fluid conduit lines 105. In some instances, the fluid conduit lines 105 are the only thing coupling the base module 130 to the breast cup 2. In some instances, the fluid conduit lines 105 are connected to the portable module coupler 104 and/or the base module coupler 136 via one or more quick-disconnect lines, such that the fluid conduit lines 105 may be switched between the portable module coupler 104 and the base module coupler 136 when switching between the portable module 106 and the base module 130.

In some embodiments, the removable pumping unit 101 is removably coupled to the base module 130 by sliding or otherwise pushing the removable pumping unit 101 into a docking slot 138 formed in the reservoir enclosure 131 and connecting the removable pump unit 101 to the base module coupler 136. For example, as best illustrated in FIG. 35, the pumping unit outlet 110 is configured to receive a pumping unit port 140 of the base module coupler 136 to fluidly couple the pumping unit outlet 110 to the fluid conduit 105, and thereby to the breast cup 2. In some instances, the docking slot 138 may be formed such that the removable pumping unit 102 snaps into the docking slot 138 and is retained within the receiving slot via the snap-in features. In some instances, the removable pumping unit 102 may additionally or alternatively be held within the docking slot 138 of the base module 130 via a latch-based connection or any other suitable releasable connection method.

As best shown in FIG. 37, the base module coupler 136 is configured to releasably and fluidly couple the pumping unit outlet 110 and a base module inlet 142 of the base module 130 to the fluid conduit lines 105 coupled to the breast cup 2. The base module coupler 136 further provides fluid communication between the base module inlet 142 and removable reservoir inlets 144 (shown in FIG. 41) of the removable reservoirs 134 via an internal fluid path 146 of a manifold 148 of the base module coupler 136 and removable reservoir connection lines 150 (shown in FIG. 39). In some embodiments, the manifold 148 further includes a control valve 152 configured to selectively direct milk from the breast cup 2 into one of a plurality of removable reservoirs 134.

Accordingly, with the removable pumping unit 101 attached to the base module 130, pressure may be selectively applied by the removable pumping unit 101 (e.g., the pressure source 107) to the pressure chamber 20 of the breast cup 2 to induce lactation from the human breast, as described herein. Further, milk pulled into the milk chamber 21, as described herein, is able to flow from or is otherwise forced out of the milk chamber 21, through the fluid conduit 105, through the base module coupler 136, and into one of the removable reservoirs 134, as directed by the control valve 152. In some instances, to allow for multiple pumping sessions, the control valve 152 may direct milk into a first removable reservoir 134 during a first pumping session and may subsequently direct milk into a second removable reservoir 134 during a second pumping session. Further, in some instances, the removable reservoir 134 not receiving milk during a pumping session may be removed from the reservoir enclosure 131 and replaced with another empty removable reservoir 134. For example, the removable reservoir connection lines 150 can be disconnected from the removable reservoir inlets 144 and the removable reservoir can be slid out of the reservoir enclosure 131.

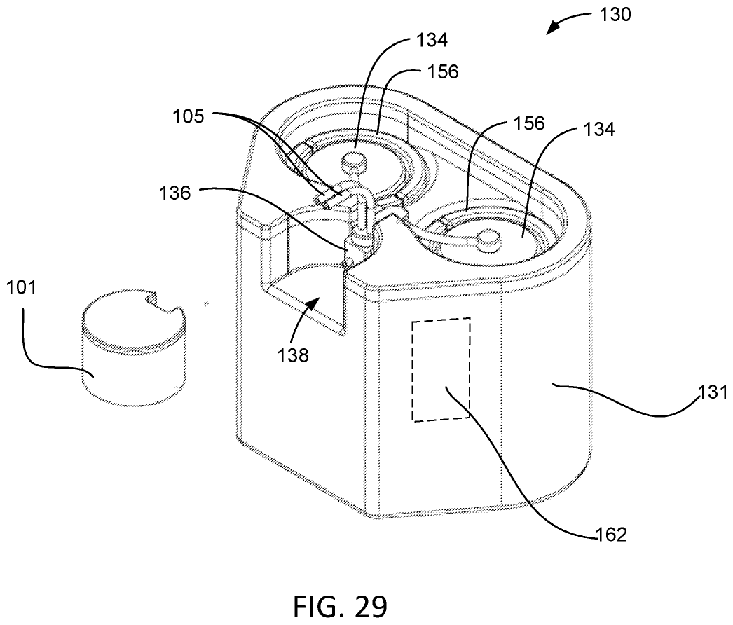

As best shown in FIGS. 29-31, 39, and 41, the reservoir enclosure 131 includes a plurality of receiving cavities 154 configured to receive the removable reservoirs 134. In some instances, each receiving cavity 154 further includes a temperature control element 156 configured to regulate a temperature of milk within a corresponding removable reservoir 134. For example, in some instances, the temperature control element 156 may be a cooling element (e.g., an ice pack, a cold pack, a frozen gel pack, a thermoelectric cooler), insulation, a heating element (e.g., a thermoelectric heater) and/or any other suitable combination of temperature control elements. In some instances, the walls of the reservoir enclosure 131 may further be insulated and/or include a layer of insulation 157 (e.g., around the receiving cavity 154).

It should be appreciated that, while the illustrated example includes a pair of receiving cavities 154 with corresponding temperature control elements 156 and removable reservoirs 134, in some instances, the reservoir enclosure 131 may include more or less of these components, as desired for a given application. For example, in some instances, the reservoir enclosure 131 may include one, three, four, five, six, seven, eight, nine, ten, or more receiving cavities 154. In these instances, the manifold 148, control valve 152, and the removable reservoir connection lines 150 may each be modified to all for milk to be selectively delivered to each of the corresponding removable reservoirs 134 individually. Further, in some instances, the reservoir enclosure 131 may not include the temperature control elements 156.

In some instances, the base module 130 may further include one or more sensors 158. For example, in some instances, one or more sensors 158 are within the removable reservoir 134 (e.g., arranged near or at a bottom of the removable reservoir 134), within the reservoir enclosure 131 that are configured to communicate various sensor data captured within the removable reservoir 134 and/or within the reservoir enclosure 131 to a processing circuit or controller 160 within the base module 130 via a wired or wireless connection. For example, in some instances, the one or more sensors 158 within the removable reservoir are configured to detect, sense, or otherwise measure one or more of a weight, a temperature, a pH, a viscosity, optical characteristics, or any other relevant information regarding milk stored within the removable reservoir 134. In some instances, one or more sensors 158 within the reservoir enclosure 131 are configured to detect, sense, or otherwise measure a temperature of the temperature control elements 156. In some instances, the base module 130 may include one or more additional sensors located in other portions of the portable module 106 (e.g., within or near the internal fluid path 146, the manifold 148, within the base module coupler 136) that are configured to sense, detect, or otherwise measure the same or additional operational characteristics, such as a volume and/or flowrate of milk flowing into the base module 130 and/or a given removable reservoir 134, and/or any other desired information pertaining to the base module 130.

In some instances, the processing circuit or controller 160 is configured to selectively activate and actuate the control valve 152 to direct milk into the appropriate removable reservoir 134. In some instances, the processing circuit or controller 160 is configured to activate and/or modify operation of the temperature control elements 156 to regulate the temperature of milk within the removable reservoirs 134 (e.g., based on temperature sensor information received from the sensors 158). For example, the processing circuit or controller 160 may include one or more processors and one or more memories storing instructions thereon that, when executed by the one or more processors, cause the one or more processing circuits to perform the various functions described herein.

In some instances, the processing circuit or controller 160 may further include one of more communication interfaces configured to allow for the processing circuit or controller 160 to communicate with other devices over a communications network (e.g., Bluetooth, Wi-Fi, near-field communication, etc.). For example, in some instances, the processing circuit or controller 160 of the base module 130 is configured to communicate with the processing circuit or controller 111 of the removable pump unit 101 to selectively activate and/or deactivate the pressure source 107 to initiate, modify, and/or stop pumping sessions (e.g., based on a removable reservoir 134 reaching a threshold fill level).

Additionally, in some instances, the processing circuit or controller 160 may include one or more input/output (I/O) devices 162 (shown in FIG. 29) configured to allow for user to selectively operate the base module 130 and/or the removable pumping unit 101 (e.g., when it is docked within the docking slot 138) and/or modify functionality parameters of the base module 130 and/or removable pumping unit 101. For example, the one or more I/O devices may include a display, one or more buttons (e.g., a power button, a pumping session start/stop button), a touchscreen, an audio speaker, a timer, an alarm, a power switch, and/or any other suitable I/O devices.

As shown in FIG. 41, the base module 130 further includes a power source 164 (e.g., a battery). In some instances, the power source 164 may be rechargeable via connection with an external power source (e.g., a wall outlet). In some other instances, the base module 130 may be configured to allow for removal and replacement of the power source 164 upon depletion of its power. In yet some other instances, the power source 164 is a power input that is connected to an external outlet.

In some instances, the power source 164 may be configured to supply power to the power source 112 of the removable pumping unit 101 when the removable pumping unit 101 is installed within the docking slot 138 of the base module 130. For example, in some instances, the docking slot 138 and removeable pumping unit 101 may include a power connection interface 165 (shown in FIG. 41), such as a physical power connector (e.g., pair of mating connectors configured to transfer power therebetween) and/or an wireless power interface (e.g., an induction-based power transfer configuration including a first induction coil within the base module 130 near the docking slot 138 and a corresponding induction coil within the removeable pumping unit 101). Accordingly, the removable pumping unit 101 may be continuously charged and/or powered via the power source 164 of the base module 130 while it is connected to or otherwise installed within the base module 130.

Accordingly, to operate the base module 130 of the modular breast pump system 1, the removable pumping unit 101 may first be placed into the docking slot 138 and coupled to the base module coupler 136, as discussed above. The removable pumping unit 101 may then be activated to apply alternating pressure (e.g., alternating negatives and nominal pressure or alternating negative and positive pressures) to the pressure chamber 20 of the breast cup 2. The alternating pressures applied to the pressure chamber 20 are similarly configured to deform the diaphragm 18 to alternatingly (i) expand the milk chamber 21 and unseal the sealing point 23 around the inlet to the milk chamber 21 to allow milk expressed from the human breast to flow into the milk chamber and (ii) contract the milk chamber 21 and seal the sealing point 23 around the inlet to the milk chamber 21 such that the expressed milk within the milk chamber 21 is forced out of the breast cup 2, through fluid conduit lines (e.g., fluid conduit lines 105), and ultimately into one of the removable reservoir 134. When a first removable reservoir 134 is filled or has hit a threshold fill level, the control valve 152 can then be actuated to begin delivering (within the same or a new pumping session) milk to the other removable reservoir 134.

In some instances, the user may then remove the removable reservoir 134 from the reservoir enclosure 131 to access the stored milk without removing the breast cup 2 from their breast. For example, the user may slide the detach the removable reservoir connection line 150 from the removable reservoir 134, slide the removable reservoir 134 out of the reservoir enclosure 131, and unscrew or otherwise remove a cap 170 (shown in FIG. 41) of the removable reservoir 134 to access the milk stored within the removable reservoir 134 while the breast cup 2 is still attached to the breast of the user.

It should be appreciated that, unlike traditional breast pumps and similar to the portable module 106 discussed above, the milk may be collected within the removable reservoir 134 of the base module 130 while the base module 130 is located away from the breast cup 2. That is, the base module 130 is connected to the breast cup 2 via fluid conduit lines (e.g., fluid conduit lines 5 or fluid conduit lines 105) that extend a distance away from the breast cup 2 (e.g., at least a foot, at least two feet, at least three feet, at least four feet) to allow for the milk to be collected off of the user's body. This off-body milk collection provides similar benefits over traditional systems as those described above, with respect to the portable module 106.

Similarly, in some instances, the diaphragm 18 returning to a nominal state may provide sufficient force onto the milk within the milk chamber 21 to force the milk out of the milk chamber 21 and into the removable reservoir 134. However, in some instances, the removable pumping unit 101 may similarly apply positive pressure to the pressure chamber 20 to further contract the milk chamber 21, and thereby force the milk out of the milk chamber 21 and into the removable reservoir 134. In some instances, negative pressure may similarly be applied from the pressure source 107 via another fluid conduit 166 (shown in FIG. 37) in the removable pumping unit 101 and base module coupler 136 to the milk chamber 21 to pull or suction the milk out of the milk chamber 21. However, to prevent a direct connection between the dry side and wet side of the pump system, a membrane diaphragm or another similar device may be utilized to apply the negative pressure from the pressure source 107 to the milk chamber 21.

In some instances, each removable reservoir 134 may similarly include a pressure outlet 168 (e.g., a pressure relieve valve) configured to allow pressurized air to escape from within the removable reservoir 134 to prevent excessive pressure build-up and/or to allow for milk to more easily flow into the removable reservoir 134.

It should be appreciated that the modular breast pump system 1 provides a versatile set of capabilities not provided by traditional breast pump systems. For example, because both the portable module and the base module are configured to utilize negative and positive pressure to induce lactation and force milk out of the breast cup, respectively, both the portable and base modules can be utilized for pumping while the user is in any position. Additionally, both modules allow for discreet pumping and off-body milk collection. In some instances, while the portable module provides a smaller form factor that can be worn by the user and is more convenient for mobile pumping, the base module may provide various complimentary functionality, such as allowing for pumping while sleeping, multiple pumping sessions to be performed consecutively, long-term (or at least quasi-long term) milk storage (e.g., due to the external power source and/or due to the temperature control elements in the reservoir enclosure), delayed pumping sessions, and/or partner-enabled pumping sessions.

### EXAMPLES

### Example 1: Exemplary Modular Breast Pump System

An exemplary modular breast pump system includes a base module and an portable module. The portable module can be used without attachment to the base module as a portable breast pump system. The base module and portable module can be used to provide serial breast pumping with milk from consecutive breast pumping sessions transported into unique reservoirs via a milk distribution system in the base module. Thus, the combined modules provide all the advantages of serial breast pumping and milk storage, with the flexibility of a fully portable breast pump via the portable module.

An exemplary portable module houses a rechargeable battery as a power source, and a pressure source that is a vacuum pump, which provides both negative and positive pressure. Three three-way solenoid valves allow for the vacuum pump to switch between negative pressure positive pressure, and atmosphere. One reservoir attaches to the portable module, such that it is easily accessible, secured, and releasable. The portable module also includes a handle or a strap that allows for ease of carrying, or wearability.

The portable module is in fluidic communication with the breast cups via the first fluid conduit and the second fluid conduit. The breast cups can be placed on the breasts of a user prior to breast pumping. Any breast cups may be used in conjunction with the portable module to provide an exemplary modular breast pump system.

The first fluid conduit and second fluid conduit also place the breast cup in fluidic communication with a reservoir in a reservoir enclosure in the portable module. In some instances, the modular breast pump system may additionally include an auxiliary reservoir enclosure (e.g., auxiliary reservoir enclosure 4 shown in FIG. 2) including a reservoir containing breast milk. The auxiliary reservoir enclosure may not be fluidically connected to the portable module or the breast cup.

Reservoirs may be manually removed from the portable module and placed in the auxiliary reservoir enclosure, e.g., a cooler, for storage.

An exemplary base module houses an exemplary milk distribution system, including a manifold and a reservoir enclosure.

The system may have a first fluidic conduit connecting the breast cup and the pressure source disposed in the portable module. The system may have a second fluidic conduit connecting the breast cup and the manifold and/or reservoir disposed in the base module.

The system may draw power from a power source housed in the base module and may charge the power source in the portable module.

The modular breast pump system includes controls that are operable by a user to select or modify at least one of: pumping program or mode, frequency of pumping cycle, maximum suction pressure achieved during a pumping cycle, latch suction pressure achieved during the pumping cycle, pumping force and pumping session time. In some instances, the controls may be accessible via an application on an external computer.

In the application, the user is able to set the pump programing including pumping length, cycle speed(s), vacuum level(s), and pump start time(s). The modular breast pump system 1 has two phases: a stimulation phase and an expression phase. The stimulation phase is typically characterized by a rapid initial stage, e.g., 100 cycles per minute, at a low to moderate vacuum strength. The initial rapid stage induces the letdown of milk and mimics the initial quick suckling motion of an infant. Once milk begins flowing, the system or the user typically switches the modular breast pump system 1 into the expression phase. Expression is typically characterized by slower cycles per minute, e.g., 38 cycles per minute, at moderate to high vacuum strength. The expression phase mimics an infant's behavior at breast with slower and deeper suckling. It is possible for the user to cycle between these two phases more than one time per session. Once a user has completed the initial stimulation and expression phase, the user may switch back to stimulation mode to produce another let down of milk and then begin another expression cycle.

As utilized herein with respect to numerical ranges, the terms “approximately,” “about,” “substantially,” and similar terms generally mean ±5% or 10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that can result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

References herein to the positions of elements (e.g., “top,” “bottom,” “above,” “below”) are merely used to describe the orientation of various elements in the FIGURES. It should be noted that the orientation of various elements can differ according to other exemplary embodiments, and that such variations are intended to be encompassed by the present disclosure. Additionally, the terms “connected” or “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

It is important to note that any element disclosed in one embodiment may be incorporated or utilized with any other embodiment disclosed herein. The systems and methods described herein may be embodied in other specific forms without departing from the characteristics thereof. The foregoing implementations are illustrative rather than limiting of the described devices and methods. The scope of the devices and methods described herein is thus indicated by the appended claims, and changes that come within the meaning and range of equivalency of the claims are embraced therein.

What is claimed is:

1. A modular breast pump system comprising:
a breast cup;
a base module including a base enclosure and a removable reservoir within the base enclosure;
a portable module including a portable reservoir; and
a removable pumping unit configured to be removably coupled with each of the base module and the portable module individually, the removable pumping unit including a pressure source in fluidic communication with the breast cup and configured to apply pressure to the breast cup that induces lactation,
wherein, when the removable pumping unit is coupled to the base module, the pressure source is configured to force milk from the breast cup into the removable reservoir and, when the removable pumping unit is coupled to the portable module, the pressure source is configured to force milk from the breast cup into the portable reservoir.

2. The modular breast pump system of claim 1, wherein the removable reservoir is one of a plurality of removable reservoirs within the base enclosure.

3. The modular breast pump system of claim 2, wherein the base module further comprises a manifold configured to selectively direct the milk to different removable reservoirs of the plurality of removable reservoirs.

4. The modular breast pump system of claim 2, wherein each removable reservoir of the plurality of removable reservoirs comprises a pressure outlet.

5. The modular breast pump system of claim 4, wherein each pressure outlet comprises a pressure relief valve.

6. The modular breast pump system of claim 1, wherein the base module includes a temperature control element configured to regulate a temperature of milk within the removable reservoir.

7. The modular breast pump system of claim 6, wherein the temperature control element comprises one or more of a cooling element configured to cool the milk within the removable reservoir or insulation.

8. The modular breast pump system of claim 1, wherein the pressure source comprises a positive pressure source and a negative pressure source, each in fluidic communication with the breast cup.

9. The modular breast pump system of claim 1, wherein the portable module further comprises a handle.

10. The modular breast pump system of claim 1, wherein the portable module further comprises one or more of a strap or a clip that allows for the portable module to be worn by a user.

11. The modular breast pump system of claim 1, further comprising a first fluid conduit providing releasable fluidic communication between the breast cup and the removable reservoir in the base enclosure of the base module or the portable reservoir of the portable module.

12. The modular breast pump system of claim 1, further comprising a second fluid conduit providing releasable fluidic communication between the pressure source and the breast cup.

13. The modular breast pump system of claim 1, wherein the breast cup comprises:
a housing;
a breast shield; and
a diaphragm disposed between a pressure chamber in fluidic communication with the pressure source and a milk chamber in fluidic communication with the removable reservoir.

14. The modular breast pump system of claim 1, further comprising one or more of a button, display, timer, alarm, or power switch.

15. The modular breast pump system of claim 1, further comprising one or more sensors including one or more of a weight sensor, a load cell, a temperature sensor, a pressure sensor, an optical sensor, a flow sensor, a volume sensor, a pH sensor, or a viscosity sensor.

16. The modular breast pump system of claim 1, wherein the portable module further comprises one or more of a cooling element or insulation.

17. The modular breast pump system of claim 1, wherein one or more of the base module or the portable module a weight sensor configured to detect an amount of milk within one or more of the removable reservoir or the portable reservoir.

18. A method of breast pumping comprising:
attaching a breast cup to a human breast of a subject;
coupling a removable pumping unit to one of a base module or a portable module of a modular breast pump system, the removable pumping unit having a pressure source;
applying pressure from the pressure source to the breast cup to pump milk from the human breast and into a first reservoir of the one of the base module or the portable module;
coupling the removable pumping unit to the other of the base module or the portable module; and
applying pressure from the pressure source to the breast cup to pump milk from the human breast and into a second reservoir of the other of the base module or the portable module.

19. The method of claim 18, wherein, when the base module is coupled to the removable pumping unit, multiple pumping sessions are performed, and the method further comprises:
actuating, between the multiple pumping sessions, a control valve within the base module to change a flow direction of the milk to flow into a third reservoir.

20. The method of claim 18, wherein, when the portable module is coupled to the removable pumping unit, the portable module is attached to the subject.
